(19) United States
(12) Patent Application Publication
(10) Pub. No.: US 2024/0327836 A1
(43) Pub. Date: Oct. 3, 2024

(54) MODIFIED OLIGONUCLEOTIDES TARGETING SNPs

(71) Applicant: UNIVERSITY OF MASSACHUSETTS, Boston, MA (US)

(72) Inventors: Anastasia Khvorova, Westborough, MA (US); Julia Alterman, Worcester, MA (US); Faith Conroy, Worcester, MA (US); Edith Pfister, Boxborough, MA (US); Neil Aronin, Newtonville, MA (US); Ken Yamada, Boston, MA (US)

(21) Appl. No.: 18/421,647

(22) Filed: Jan. 24, 2024

Related U.S. Application Data
(63) Continuation of application No. 16/988,391, filed on Aug. 7, 2020, now Pat. No. 12,024,706.
(60) Provisional application No. 62/976,168, filed on Feb. 13, 2020.
(60) Provisional application No. 62/885,066, filed on Aug. 9, 2019.

(51) Int. Cl. C12N 15/113

(52) U.S. Cl. CPC C12N 15/113; C12N 2310/14; C12N 2310/52

(57) ABSTRACT

Novel oligonucleotides that enhance silencing of the expression of a gene containing a single nucleotide polymorphism (SNP) relative to the expression of the corresponding wild-type gene are provided. Methods of using novel oligonucleotides that enhance silencing of the expression of a gene containing a SNP relative to the expression of the corresponding wild-type gene are provided.

### RELATED APPLICATIONS

This application is a continuation of U.S. patent application Ser. No. 16/988,391, filed Aug. 7, 2020, which claims priority to U.S. Provisional Patent Application Nos. 62/885,066, filed Aug. 9, 2019, and 62/976,168, filed Feb. 13, 2020. The entire contents of these applications are incorporated herein by reference.

### STATEMENT OF FEDERALLY SPONSORED RESEARCH

This invention was made with government support under Grant Nos. NS104022 and GM108803 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML file, created on Jan. 24, 2024, is named 749626_UM9-227CIPCON_ST26.xml and is 501,561 bytes in size.

### BACKGROUND

RNA interference represents a simple and effective tool for inhibiting the function of genes. RNA silencing agents have received particular interest as research tools and therapeutic agents for their ability to knock down expression of a particular protein with a high degree of sequence specificity. The sequence specificity of RNA silencing agents is particularly useful for the treatment of diseases caused by dominant mutations in heterozygotes bearing one mutant and one wild-type copy of a particular gene. However, there remains a need for RNA silencing agents that can preferentially silence mutant, disease-causing allele expression while not (or only minimally) effecting expression of the wild-type allele.

### SUMMARY

The present disclosure is based, at least in part, on the surprising discovery of novel oligonucleotides that enhance silencing of the expression of a gene containing a single nucleotide polymorphism (SNP) (e.g., a heterozygous SNP) relative to the expression of the corresponding wild-type gene in a heterozygote, e.g., by up to more than 100-fold. In certain aspects, an oligonucleotide (e.g., a dsRNA) is provided that preferentially targets a SNP-containing nucleic acid for degradation, wherein the oligonucleotide (e.g., a double-stranded RNA (dsRNA)) does not target, or targets to a lesser degree, the corresponding wild-type (non-SNP-containing) nucleic acid for degradation. In certain aspects, an oligonucleotide (e.g., a dsRNA) of the disclosure is: 1) complementary to a SNP position in a target nucleic acid; and 2) contains a mismatch at a particular position of the target nucleic acid relative to the SNP. In certain embodiments, an oligonucleotide (e.g., a dsRNA) contains two mismatches relative to the corresponding wild-type target nucleic acid sequence: 1) at the wild-type SNP position; and 2) at the particular position of the target nucleic acid sequence relative to the wild-type SNP position. Accordingly, an exemplary oligonucleotide (e.g., dsRNA) contains one mismatch relative to a SNP-containing target and two mismatches relative to the corresponding wild-type sequence, thus resulting in preferential cleavage of the SNP-containing target relative to the corresponding wild-type sequence.

In one aspect, a nucleic acid having a 5′ end, a 3′ end and a seed region, that is complementary to a region of a gene comprising an allelic polymorphism, wherein the nucleic acid comprises a single nucleotide polymorphism (SNP) position nucleotide at a position within the seed region, wherein the SNP position nucleotide is complementary to the allelic polymorphism, a mismatch (MM) position that is a mismatch with a nucleotide in the gene, and at least one modified nucleotide (X) on either side of the SNP position nucleotide, wherein each X is located within four, three or two nucleotides from the SNP position nucleotide, is provided. In some embodiments, each X is located within four nucleotides from the SNP position nucleotide. In some embodiments, each X is located within three nucleotides from the SNP position nucleotide In some embodiments, each X is located within two nucleotides from the SNP position nucleotide.

In certain exemplary embodiments, each X independently comprises a sugar modification selected from the group consisting of 2′-O-methyl (2′-OMe), 2′-fluoro (2′-F), 2′-ribo, 2′-deoxyribo, 2′-F-4′-thioarabino (2′-F-ANA), 2′-O-(2-methoxyethyl) (2′-MOE), 4′-S-RNA, locked nucleic acid (LNA), 4′-S-F-ANA, 2′-O-allyl, 2′-O-ethylamine, 2′-O-cyanoethyl-RNA (CNet-RNA), tricyclo-DNA, cyclohexenyl nucleic acid (CeNA), arabino nucleic acid (ANA), and hexitol nucleic acid (HNA).

In certain exemplary embodiments, an X is positioned immediately 5′ to the SNP position nucleotide or immediately 3′ to the SNP position nucleotide. In certain exemplary embodiments, an X is positioned immediately 5′ to the SNP position nucleotide and immediately 3′ to the SNP position nucleotide.

In certain exemplary embodiments, the SNP position nucleotide is present at from position 2 to position 6 from the 5′ end (e.g., at position 2 from the 5′ end, at position 3 from the 5′ end, at position 4 from the 5′ end, at position 5 from the 5′ end, or at position 6 from the 5′ end). In certain exemplary embodiments, the SNP position nucleotide is present at from position 3 to position 6 from the 5′ end (e.g., at position 3 from the 5′ end, at position 4 from the 5′ end, at position 5 from the 5′ end, or at position 6 from the 5′ end). In some embodiments, the SNP position nucleotide is present at from position 4 to position 6 from the 5′ end (e.g., at position 4 from the 5′ end, at position 5 from the 5′ end, or at position 6 from the 5′ end). In some embodiments, the SNP position nucleotide is present at from position 2 to position 5 from the 5′ end (e.g., at position 2 from the 5′ end, at position 3 from the 5′ end, at position 4 from the 5′ end, or at position 5 from the 5′ end). In some embodiments, the SNP position nucleotide is present at from position 3 to position 5 from the 5′ end (e.g., at position 3 from the 5′ end, at position 4 from the 5′ end, or at position 5 from the 5′ end). In some embodiments, the SNP position nucleotide is present at from position 2 to position 4 from the 5′ end (e.g., at position 2 from the 5′ end, at position 3 from the 5′ end, or at position 4 from the 5′ end).

In some embodiments, the MM position nucleotide is located from 2 to 11 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide. In some embodiments, the MM position is located 7 nucleotides from the SNP position nucleotide. In some embodiments, the MM position is located 8 nucleotides from the SNP position nucleotide. In some embodiments, the MM position is located 9 nucleotides from the SNP position nucleotide. In some embodiments, the MM position is located 10 nucleotides from the SNP position nucleotide. In some embodiments, the MM position is located 11 nucleotides from the SNP position nucleotide.

In some embodiments, the MM position nucleotide is located from 2 to 6 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide.

In another aspect, a nucleic acid having a 5′ end, a 3′ end and a seed region, that is complementary to a region of a gene comprising an allelic polymorphism, wherein the nucleic acid comprises a SNP position nucleotide at a position within the seed region, wherein the SNP position nucleotide is complementary to the allelic polymorphism, a MM position that is a mismatch with a nucleotide in the gene, and at least one modified nucleotide (Y) on either side of the MM position nucleotide, wherein each Y is located within four, three or two nucleotides from the MM position nucleotide, is provided. In some embodiments, each Y is located within four nucleotides from the MM position nucleotide. In some embodiments, each Y is located within three nucleotides from the MM position nucleotide. In some embodiments, each Y is located within two nucleotides from the MM position nucleotide.

In some embodiments, each Y independently comprises a sugar modification selected from the group consisting of 2′-OMe, 2′-F, 2′-ribo, 2′-deoxyribo, 2′-F-ANA, 2′-MOE, 4′-S-RNA, LNA, 4′-S-F-ANA, 2′-O-allyl, 2′-O-ethylamine, CNet-RNA, tricyclo-DNA, CeNA, ANA, and HNA.

In some embodiments, a Y is positioned immediately 5′ to the MM position nucleotide or immediately 3′ to the MM position nucleotide. In certain exemplary embodiments, a Y is positioned immediately 5′ to the MM position nucleotide and immediately 3′ to the MM position nucleotide.

In certain exemplary embodiments, the SNP position nucleotide is present at position 2 to position 6 from the 5′ end (e.g., at position 2 from the 5′ end, at position 3 from the 5′ end, at position 4 from the 5′ end, at position 5 from the 5′ end, or at position 6 from the 5′ end). In some embodiments, the SNP position nucleotide is present at from position 3 to position 6 from the 5′ end (e.g., at position 3 from the 5′ end, at position 4 from the 5′ end, at position 5 from the 5′ end, or at position 6 from the 5′ end). In some embodiments, the SNP position nucleotide is present at from position 4 to position 6 from the 5′ end (e.g., at position 4 from the 5′ end, at position 5 from the 5′ end, or at position 6 from the 5′ end). In some embodiments, the SNP position nucleotide is present at from position 2 to position 5 from the 5′ end (e.g., at position 2 from the 5′ end, at position 3 from the 5′ end, at position 4 from the 5′ end, or at position 5 from the 5′ end). In some embodiments, the SNP position nucleotide is present at from position 3 to position 5 from the 5′ end (e.g., at position 3 from the 5′ end, at position 4 from the 5′ end, or at position 5 from the 5′ end). In some embodiments, the SNP position nucleotide is present at from position 2 to position 4 from the 5′ end (e.g., at position 2 from the 5′ end, at position 3 from the 5′ end, or at position 4 from the 5′ end).

In some embodiments, the MM position nucleotide is located from 2 to 11 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide. In some embodiments, the MM position is located 7 nucleotides from the SNP position nucleotide. In some embodiments, the MM position is located 8 nucleotides from the SNP position nucleotide. In some embodiments, the MM position is located 9 nucleotides from the SNP position nucleotide. In some embodiments, the MM position is located 10 nucleotides from the SNP position nucleotide. In some embodiments, the MM position is located 11 nucleotides from the SNP position nucleotide.

In some embodiments, the MM position nucleotide is located from 2 to 6 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide.

In another aspect, a nucleic acid having a 5′ end, a 3′ end and a seed region, that is complementary to a region of a gene comprising an allelic polymorphism, wherein the nucleic acid comprises a SNP position nucleotide at a position within the seed region, wherein the SNP position nucleotide is complementary to the allelic polymorphism, a MM position that is a mismatch with a nucleotide in the gene, and at least one modified nucleotide (X) on either side of the SNP position nucleotide, wherein each X is located within four, three or two nucleotides from the SNP position nucleotide, and at least one modified nucleotide (Y) on either side of the MM position nucleotide, wherein each Y is located within four, three or two nucleotides from the MM position nucleotide, is provided. In some embodiments, each X is located within four nucleotides from the SNP position nucleotide. In some embodiments, each X is located within three nucleotides from the SNP position nucleotide. In some embodiments, each X is located within two nucleotides from the SNP position nucleotide. In some embodiments, each Y is located within four nucleotides from the MM position nucleotide. In some embodiments, each Y is located within three nucleotides from the MM position nucleotide. In some embodiments, each Y is located within two nucleotides from the MM position nucleotide.

In some embodiments, each X independently comprises a sugar modification selected from the group consisting of 2′-OMe, 2′-F, 2′-ribo, 2′-deoxyribo, 2′-F-ANA, 2′-MOE, 4′-S-RNA, LNA, 4′-S-F-ANA, 2′-O-allyl, 2′-O-ethylamine, CNet-RNA, tricyclo-DNA, CeNA, ANA, and HNA. In some embodiments, each Y independently comprises a sugar modification selected from the group consisting of 2′-OMe, 2′-F, 2′-ribo, 2′-deoxyribo, 2′-F-ANA, 2′-MOE, 4′-S-RNA, LNA, 4′-S-F-ANA, 2′-O-allyl, 2′-O-ethylamine, CNet-RNA, tricyclo-DNA, CeNA, ANA, and HNA.

In certain exemplary embodiments, an X is positioned immediately 5′ to the SNP position nucleotide or immediately 3′ to the SNP position nucleotide. In certain exemplary embodiments, an X is positioned immediately 5′ to the SNP position nucleotide and immediately 3′ to the SNP position nucleotide.

In certain exemplary embodiments, a Y is positioned immediately 5′ to the MM position nucleotide or immediately 3′ to the MM position nucleotide. In certain exemplary embodiments, a Y is positioned immediately 5′ to the MM position nucleotide and immediately 3′ to the MM position nucleotide.

In certain exemplary embodiments, the SNP position nucleotide is present from position 2 to position 6 from the 5′ end (e.g., at position 2 from the 5′ end, at position 3 from the 5′ end, at position 4 from the 5′ end, at position 5 from the 5′ end, or at position 6 from the 5′ end). In some embodiments, the SNP position nucleotide is present at from position 3 to position 6 from the 5′ end (e.g., at position 3 from the 5′ end, at position 4 from the 5′ end, at position 5 from the 5′ end, or at position 6 from the 5′ end). In some embodiments, the SNP position nucleotide is present at from position 4 to position 6 from the 5′ end (e.g., at position 4 from the 5′ end, at position 5 from the 5′ end, or at position 6 from the 5′ end). In some embodiments, the SNP position nucleotide is present at from position 2 to position 5 from the 5′ end (e.g., at position 2 from the 5′ end, at position 3 from the 5′ end, at position 4 from the 5′ end, or at position 5 from the 5′ end). In some embodiments, the SNP position nucleotide is present at from position 3 to position 5 from the 5′ end (e.g., at position 3 from the 5′ end, at position 4 from the 5′ end, or at position 5 from the 5′ end). In some embodiments, the SNP position nucleotide is present at from position 2 to position 4 from the 5′ end (e.g., at position 2 from the 5′ end, at position 3 from the 5′ end, or at position 4 from the 5′ end).

In some embodiments, the MM position nucleotide is located from 2 to 11 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide. In some embodiments, the MM position is located 7 nucleotides from the SNP position nucleotide. In some embodiments, the MM position is located 8 nucleotides from the SNP position nucleotide. In some embodiments, the MM position is located 9 nucleotides from the SNP position nucleotide. In some embodiments, the MM position is located 10 nucleotides from the SNP position nucleotide. In some embodiments, the MM position is located 11 nucleotides from the SNP position nucleotide.

embodiments, the MM position nucleotide is located from 2 to 6 nucleotides from the SNP position nucleotide. For example, in some embodiments, the MM position is located 2 nucleotides from the SNP position nucleotide. In some embodiments, the MM position is located 3 nucleotides from the SNP position nucleotide. In some embodiments, the MM position is located 4 nucleotides from the SNP position nucleotide. In some embodiments, the MM position is located 5 nucleotides from the SNP position nucleotide. In some embodiments, the MM position is located 6 nucleotides from the SNP position nucleotide.

In some embodiments, X and Y are the same.

In another aspect, a nucleic acid having a 5′ end and a 3′ end that is complementary to a region of a gene comprising an allelic polymorphism, wherein the nucleic acid comprises a SNP position nucleotide that is complementary to the allelic polymorphism, a MM position nucleotide that is a mismatch with a nucleotide in the gene, at least one 2′-fluoro-ribonucleotide on either side of the SNP position nucleotide, wherein each 2′-fluoro-ribonucleotide is located within four, three or two nucleotides from the SNP position nucleotide, and at least one 2′-methoxy-ribonucleotide on either side of the MM position nucleotide, wherein each 2′-methoxy-ribonucleotide is located within four, three or two nucleotides from the MM position nucleotide, is provided.

In certain exemplary embodiments, a 2′-fluoro-ribonucleotide is positioned immediately 5′ to the SNP position nucleotide or a 2′-fluoro-ribonucleotide is positioned immediately 3′ to the SNP position nucleotide.

In certain exemplary embodiments, a 2′-fluoro-ribonucleotide is positioned immediately 5′ to the SNP position nucleotide and a 2′-fluoro-ribonucleotide is positioned immediately 3′ to the SNP position nucleotide.

In certain exemplary embodiments, a 2′-methoxy-ribonucleotide is positioned immediately 5′ to the MM position nucleotide or a 2′-methoxy-ribonucleotide is positioned immediately 3′ to the MM position nucleotide. In certain exemplary embodiments, a 2′-methoxy-ribonucleotide is positioned immediately 5′ to the MM position nucleotide and a 2′-methoxy-ribonucleotide is positioned immediately 3′ to the MM position nucleotide.

In certain exemplary embodiments, the SNP position nucleotide is present in a seed region, and the MM position nucleotide is located from 2 to 11 nucleotides from the SNP position nucleotide (e.g., 2 nucleotides from the SNP position nucleotide, 3 nucleotides from the SNP position nucleotide, 4 nucleotides from the SNP position nucleotide, 5 nucleotides from the SNP position nucleotide, 6 nucleotides from the SNP position nucleotide, 7 nucleotides from the SNP position nucleotide, 8 nucleotides from the SNP position nucleotide, 9 nucleotides from the SNP position nucleotide, 10 nucleotides from the SNP position nucleotide, or 11 nucleotides from the SNP position nucleotide).

In certain exemplary embodiments, the SNP position nucleotide is present from position 2 to position 6 from the 5′ end, and the MM position nucleotide is located 2-6 nucleotides from the SNP position nucleotide.

In certain exemplary embodiments, the nucleic acid comprises three, four, five or six 2′-fluoro-ribonucleotides. In certain exemplary embodiments, the nucleic acid comprises three, four, five or six 2′-methoxy-ribonucleotides.

In another aspect, a nucleic acid having a 5′ end and a 3′ end that is complementary to a region of a gene comprising an allelic polymorphism, wherein the nucleic acid comprises a SNP position nucleotide that is complementary to the allelic polymorphism, a MM position nucleotide that is a mismatch with a nucleotide in the gene, at least three 2′-fluoro-ribonucleotides located within four, three or two nucleotides from the SNP position nucleotide, and at least three 2′-methoxy-ribonucleotides located within four, three or two nucleotides from the MM position nucleotide, is provided.

In certain exemplary embodiments, a 2′-fluoro-ribonucleotide is positioned immediately 5′ to the SNP position nucleotide or a 2′-fluoro-ribonucleotide is positioned immediately 3′ to the SNP position nucleotide. In certain exemplary embodiments, a 2′-fluoro-ribonucleotide is positioned immediately 5′ to the SNP position nucleotide and a 2′-fluoro-ribonucleotide is positioned immediately 3′ to the SNP position nucleotide.

In certain exemplary embodiments, a 2′-methoxy-ribonucleotide is positioned immediately 5′ to the MM position nucleotide or a 2′-methoxy-ribonucleotide is positioned immediately 3′ to the MM position nucleotide.

In certain exemplary embodiments, a 2′-methoxy-ribonucleotide is positioned immediately 5′ to the MM position nucleotide and a 2′-methoxy-ribonucleotide is positioned immediately 3′ to the MM position nucleotide.

In certain exemplary embodiments, the SNP position nucleotide is present in a seed region, and the MM position nucleotide is located 2-11 nucleotides from the SNP position nucleotide.

In certain exemplary embodiments, the SNP position nucleotide is present from position 2 to position 6 from the 5′ end, and wherein the MM position nucleotide is located 2-6 nucleotides from the SNP position nucleotide.

In a further aspect, an siRNA molecule comprises a sense strand having complementarity to a target gene and an antisense strand having complementarity to the sense strand, wherein the antisense strand comprises the nucleic acid of any one of the foregoing aspects or embodiments, is provided.

In some embodiments, the sense strand has a length of from 13 nucleotides or nucleotide analogs to 17 nucleotides or nucleotide analogs (e.g., a length of 13, 14, 15, 16, or 17 nucleotides or nucleotide analogs).

In some embodiments, the antisense strand has a length of from 18 nucleotides or nucleotide analogs to 22 nucleotides or nucleotide analogs (e.g., a length of 18, 19, 20, 21, or 22 nucleotides or nucleotide analogs).

In some embodiments, the sense strand has a length of 15 nucleotides or nucleotide analogs and the antisense strand has a length of 20 nucleotides or nucleotide analogs.

In some embodiments, the sense strand has a length of 16 nucleotides or nucleotide analogs and the antisense strand has a length of 20 nucleotides or nucleotide analogs.

In a further aspect, a branched oligonucleotide comprising two or more siRNA molecules covalently bound to one another, wherein each siRNA molecule is, independently, an siRNA molecule of any one of the preceding aspects or embodiments, is provided.

In some embodiments, the branched oligonucleotide comprises two of the siRNA molecules covalently bound to one another.

In some embodiments, the siRNA molecules are covalently bound to one another by way of a linker.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts psiCHECK reporter plasmids containing either a wild-type region of htt or the same region of htt with the SNP, rs362273. FIG. 1 discloses SEQ ID NOS 265-268, respectively, in order of appearance.

FIG. 2 depicts a bar graph showing luciferase activity following a psiCHECK reporter plasmid assay in Hela cells transfected with hsiRNAs with the SNP nucleotide at varying positions. This primary screen yielded multiple efficacious hydrophobically modified siRNA (hsiRNA) sequences.

FIG. 3 depicts dose response curves showing the silencing effects of three exemplary hsiRNAs on psiCHECK reporter plasmids.

FIG. 4 depicts a dose response curve showing the efficacy of two hsiRNAs on silencing htt mRNA.

FIG. 5 depicts bar graphs showing luciferase activity following a psiCHECK reporter plasmid assay in Hela cells transfected with hsiRNAs having a second mismatch at varying positions.

FIG. 6 depicts dose response curves comparing silencing effects of SNP2 hsiRNA with (mm2-7) or without (mm2-0) an additional mismatch.

FIG. 7 depicts dose response curves comparing silencing effects of SNP4 hsiRNAs with or without an additional mismatch.

FIG. 8 depicts dose response curves comparing silencing effects of SNP6 hsiRNAs with or without an additional mismatch.

FIG. 9 depicts dose response curves comparing silencing effects of SNP4 or SNP6 hsiRNAs with an additional mismatch (SNP4-7 and SNP6-11, respectively), compared to the same hsiRNAs without an additional mismatch (SNP4-0 and SNP4-11). HeLa cells transfected with one of two reporter plasmids were revers transfected with hsiRNAs by passive uptake, and treated for 72 hours. reporter expression was measured with a dual luciferase assay.

FIG. 10 depicts dose response curves of htt mRNA expression that measures silencing efficacy of hsiRNAs with additional mismatches.

FIG. 11 schematically depicts an hsiRNA and exemplary modifications according to some embodiments.

FIG. 12A-FIG. 12C depict the SNP2, SNP4 and SNP6 hsiRNA libraries, respectively. Antisense strands are depicted 5′ to 3′, with the SNP site in red and the mismatch in blue. FIG. 12A discloses SEQ ID NOS 269-284, respectively, in order of appearance. FIG. 12B discloses SEQ ID NOS 285-300, respectively, in order of appearance. FIG. 12C discloses SEQ ID NOS 301-316, respectively, in order of appearance.

FIG. 13 depicts antisense and sense strand sequences and modification patterns for various hsiRNA constructs according to certain embodiments. mm4-7 and mm6-11 demonstrated superior SNP discrimination, and were selected for further screening. FIG. 13 discloses SEQ ID NOS 317, 291-292, 299, 305, 308, 311, 314, 316, 318, 319, 319, 319, 320, 320, 320, 320 and 320, respectively, in order of columns.

FIG. 14 depicts an exemplary SNP-selective compound designed as a di-siRNA. FIG. 14 discloses SEQ ID NOS 321-322 and 321-322, respectively, in order of appearance.

FIG. 15 depicts backbone linkages according to certain exemplary embodiments. Oligonucleotide backbones may comprise one or any combination of phosphates, phosphorothioates (a racemic mixture or stereospecific), diphosphorothioates, phosphoramidates, acids (PNAs), boranophosphates, 2′-5′-phosphodiesters, amides, peptide nucleic phosphonoacetates, morpholinos and the like

FIG. 16 depicts sugar modifications according to certain exemplary embodiments. Sugar modifications include one or any combination of 2′-O-methyl, 2′-fluoro, 2′-ribo, 2′-deoxyribo, 2′-F-ANA, MOE, 4′-S-RNA, LNA, 4′-S-F-ANA, 2′-O-allyl, 2′-O-ethylamine, CNet-RNA, tricyclo-DNA, CeNA, ANA, HNA and the like.

FIG. 17 depicts internucleotide bonds according to certain exemplary embodiments. Potential internucleotide bonds can be between the first two nucleotides at the 5′ or 3′ ends of any given oligonucleotide strand can be stabilized with any of the moieties depicted.

FIG. 18 depicts 5′ stabilization modifications according to certain exemplary embodiments. A suitable 5′ stabilization modification can be a phosphate, no phosphate, a vinyl phosphonate, a C5-methyl (R or S or racemic), a C5-methyl on vinyl, reduced vinyl (e.g., three carbon alkyl) or the like.

FIG. 19 depicts conjugates moieties according to certain exemplary embodiments. A suitable conjugated moiety can be any length alkyl chain, a vitamin, a ligand, a peptide or a bioactive conjugate, e.g., a glycosphingolipid, a polyunsaturated fatty acid, a secosteroid, a steroid hormone, a steroid lipid, or the like.

FIG. 20 graphically depicts that the activity of a SNP discriminating scaffold that comprises a SNP position nucleotide at position 6 from the 5′ end, and a mismatch position nucleotide located at position 11 from the 5′ end, is sequence-independent.

FIG. 21 illustrates a representative synthesis of the vinyl phosphonate (VP)-modified intersubunit linkage described herein.

FIG. 22 depicts a method for preparing oligonucleotides having a VP-modified intersubunit linkage.

FIG. 23 is a pictoral representation of a VP-modified RNA according to certain exemplary embodiments.

FIG. 24 illustrates the sequences of VP-modified oligonucleotides synthesized according to certain exemplary embodiments. FIG. 24 discloses SEQ ID NOS 323-342, respectively, in order of appearance.

FIG. 25 is a summary of a comparative study of siRNA efficacy.

FIG. 26 is a schematic of hsiRNA antisense scaffolds aligned to HTT sequence surrounding SNP site rs362273 wherein the green box depicts the position of the SNP site.

FIGS. 27A and 27B illustrate the effect of adding a mismatch in the siRNA sequence improves allelic discrimination without impairing the silencing of the mutant allele. FIG. 27A discloses SEQ ID NOS 343-346, 234, 347-348, 235, 349-350, 236, 351-352, 237, 353 and 238, respectively, in order of appearance.

FIGS. 28A and 28B depict VP-modified sequences prepared by a synthesizer. FIG. 28A discloses SEQ ID NOS 354-360, respectively, in order of appearance. FIG. 28B discloses SEQ ID NOS 361-364, 355-360, respectively, in order of appearance.

FIG. 29 demonstrates another method for preparing the VP-modified oligonucleotides provided herein.

FIG. 30 demonstrates the effect a VP-modified linkage has on target/non-target discrimination of SNP-selective siRNAs.

FIG. 31 illustrates an example di-branched siRNA chemical scaffold.

FIG. 32A is a western blot performed to measure HTT protein levels. FIG. 32B shows protein levels normalized to vinculin.

FIG. 33 depicts dose response curves comparing silencing effects for oligonucleotides targeting G at the SNP site instead of A.

FIG. 34 illustrates example sequences introducing single mismatches in sequences previously chosen for dose response. FIG. 34 discloses SEQ ID NOS 343-346, 234, 348, 235, 349-350, 236, 351-352, 237, 353, 238, respectively, in order of appearance.

FIG. 35 illustrates a number of exemplary oligonucleotide backbone modifications.

FIG. 35 discloses SEQ ID NOS 365, 386 and 366-373, respectively, in order of columns.

FIG. 36 shows oligonucleotide branching motifs according to certain exemplary embodiments. The double-helices represent oligonucleotides. The combination of different linkers, spacer(s) and branching points allows generation of a wide diversity of branched hsiRNA structures.

FIG. 37 shows exemplary branched oligonucleotides with conjugated bioactive moieties.

FIG. 38 shows exemplary amidite linkers, spacers and branching moieties.

FIG. 39 is a schematic of hsiRNA antisense scaffolds aligned to HTT sequence surrounding alternative SNP site rs362273.

FIG. 40 depicts bar graphs showing luciferase activity following a psiCHECK reporter plasmid assay in HeLa cells transfected with the hsiRNAs of FIG. 39. The number following “SNP” represents the position of the SNP in the siRNA.

FIG. 41 depicts dose response curves comparing silencing effects for oligonucleotides of FIG. 39 targeting C or T at the SNP3 site.

FIG. 42 depicts bar graphs showing luciferase activity following a psiCHECK reporter plasmid assay in Hela cells transfected with hsiRNAs of FIG. 39 which were modified to feature a second mismatch at varying positions.

FIG. 43 illustrates example modified intersubunit linkers.

FIG. 44A shows a representative example for preparing a monomer for the modified phosphinate-containing oligonucleotides provided herein. FIG. 44B shows a representative example for preparing another monomer for the modified phosphinate-containing oligonucleotides provided herein. FIG. 44C shows a representative example for preparing a modified phosphinate-containing oligonucleotides provided herein.

FIG. 45 illustrates exemplary SNPs within the HTT gene (SEQ ID NOs: 1-10 (numbered from top to bottom)).

FIG. 46 is a flow chart illustrating a methodology for generating and selecting SNP-discriminating siRNAs.

FIG. 47 illustrates a naming convention denoting the position of an SNP within an siRNA. FIG. 47 discloses SEQ ID NOS 374-385, respectively, in order of appearance.

FIG. 48A-FIG. 48D graphically depict efficacy and discrimination of target and non-target binding and cleavage mediated by altering the 2′-fluoro/2′-methoxy content adjacent to the SNP position nucleotide and the MM position nucleotide of an siRNA. FIG. 48A depicts results for a variety of SNP 6-11 variants. FIG. 48B depicts results for 6-11 vs. 6-11 having a modified ffmff pattern (four 2′-fluoro-ribonucleotides and a 2′-methoxy-ribonucleotide near the SNP at position 6). FIG. 48C depicts results for 6-11 vs. 6-11 having a modified mmm11 pattern (three 2′-methoxy-ribonucleotides near the MM at position 11). FIG. 48D depicts results for 6-11 vs. 6-11 having a modified fff6mmm11 pattern (three 2′-fluoro-ribonucleotides near the SNP at position 6, and three 2′-methoxy-ribonucleotides near the MM at position 11). HeLa cells were transfected with one of two reporter plasmids, were reverse-transfected with siRNAs by passive uptake, and treated for 72 hours. Reporter expression was measured using a dual-luciferase assay.

### DETAILED DESCRIPTION

The present disclosure relates to compositions comprising oligonucleotide, e.g., RNA, silencing agents, e.g., RNAs such as double-stranded RNAs (“dsRNAs”), antisense oligonucleotides (“ASOs”) and the like, that are useful for silencing allelic polymorphisms located within a gene encoding a mutant protein. In a particular aspect, an oligonucleotide, e.g., an RNA, silencing agent is a dsRNA agent provided herein, that destroys a corresponding mutant mRNA (e.g., a SNP-containing mRNA) with nucleotide specificity and selectivity. Oligonucleotide, e.g., RNA, silencing agents, e.g., dsRNA agents disclosed herein target mRNA corresponding to polymorphic regions of a mutant gene, resulting in cleavage of mutant mRNA, and preventing synthesis of the corresponding mutant protein e.g., a gain of function mutant protein, such as the huntingtin protein.

### Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof),

As used herein, the term “capping group” refers to a chemical moiety that replaces a hydrogen atom in a functional group such as an alcohol (ROH), a carboxylic acid (RCO₂H), or an amine (RNH₂). Non-limiting examples of capping groups include: alkyl (e.g., methyl, tertiary-butyl); alkenyl (e.g., vinyl, allyl); carboxyl (e.g., acetyl, benzoyl); carbamoyl; phosphate; and phosphonate (e.g., vinylphosphonate). Other suitable capping groups are known to those of skill in the art.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino) propyl uridine, 5-bromo uridine, 5-propyne uridine, 5-propenyl uridine, and the like; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4): 297-310.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. In particular embodiments, RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, exemplary oligonucleotides include, but are not limited to, siRNAs, miRNAs, shRNAs, CRISPR guides, DNA oligonucleotides, antisense oligonucleotides, AAV oligonucleotides, gapmers, mixmers, miRNA inhibitors, SSOs, PMOs, PNAs and the like.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

As used herein, the term “hsiRNA” refers to an embodiment of the double-stranded RNAs provided herein, wherein the RNA molecule is fully chemically modified, including one or more hydrophobic modifications, as described herein.

An RNAi agent, e.g., an RNA silencing agent, having a strand which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, translational repression and the like) mediated by RNA molecules, which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence, e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g., promoter or enhancer elements) of a target gene. In other embodiments, the target polynucleotide sequence is a target mRNA encoded by a target gene.

A gene “involved” in a disease or disorder includes a gene, the normal or aberrant expression or function of which effects or causes the disease or disorder or at least one symptom of said disease or disorder.

As used herein, the term “target gene” (e.g., the mutant allele of a heterozygous polymorphism, e.g., a heterozygous SNP) is a gene whose expression is to be substantially inhibited or “silenced.” This silencing can be achieved by RNA silencing, e.g., by cleaving the mRNA of the target gene or translational repression of the target gene. The term “non-target gene” (e.g., the wild-type allele) is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., single nucleotide polymorphisms or SNPs). In another embodiment, the target and non-target genes can share less than 100% sequence identity. In another embodiment, the non-target gene may be a homolog (e.g., an ortholog or paralog) of the target gene.

A “target allele” is an allele (e.g., a SNP allele) whose expression is to be selectively inhibited or “silenced.” This silencing can be achieved by RNA silencing, e.g., by cleaving the mRNA of the target gene or target allele by a siRNA. The term “non-target allele” is an allele (e.g., the corresponding wild-type allele) whose expression is not to be substantially silenced. In certain embodiments, the target and non-target alleles can correspond to the same target gene. In other embodiments, the target allele corresponds to, or is associated with, a target gene, and the non-target allele corresponds to, or is associated with, a non-target gene. In one embodiment, the polynucleotide sequences of the target and non-target alleles can differ by one or more nucleotides. In another embodiment, the target and non-target alleles can differ by one or more allelic polymorphisms (e.g., one or more SNPs). In another embodiment, the target and non-target alleles can share less than 100% sequence identity.

The term “polymorphism,” as used herein, refers to a variation (e.g., one or more deletions, insertions, or substitutions) in a gene sequence that is identified or detected when the same gene sequence from different sources or subjects (but from the same organism) are compared. For example, a polymorphism can be identified when the same gene sequence from different subjects are compared. Identification of such polymorphisms is routine in the art, the methodologies being similar to those used to detect, for example, breast cancer point mutations. Identification can be made, for example, from DNA extracted from a subject's lymphocytes, followed by amplification of polymorphic regions using specific primers to said polymorphic region. Alternatively, the polymorphism can be identified when two alleles of the same gene are compared.

In particular embodiments, the polymorphism is a single nucleotide polymorphism (SNP). A variation in sequence between two alleles of the same gene within an organism is referred to herein as an “allelic polymorphism.” In certain embodiments, the allelic polymorphism corresponds to a SNP allele. For example, the allelic polymorphism may comprise a single nucleotide variation between the two alleles of a SNP, also referred to herein as a heterozygous SNP. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene. In particular embodiments, the polymorphism is found in a coding region of the gene or in an untranslated region (e.g., a 5′ UTR or 3′ UTR) of the gene.

As used herein, the term “allelic frequency” is a measure (e.g., proportion or percentage) of the relative frequency of an allele (e.g., a SNP allele) at a single locus in a population of individuals. For example, where a population of individuals carry n loci of a particular chromosomal locus (and the gene occupying the locus) in each of their somatic cells, then the allelic frequency of an allele is the fraction or percentage of loci that the allele occupies within the population. In particular embodiments, the allelic frequency of an allele (e.g. a SNP allele) is at least 10% (e.g., at least 15%, 20%, 25%, 30%, 35%, 40% or more) in a sample population.

The term “gain-of-function mutation,” as used herein, refers to any mutation in a gene in which the protein encoded by said gene (i.e., the mutant protein) acquires a function not normally associated with the protein (i.e., the wild-type protein) causes or contributes to a disease or disorder. The gain-of-function mutation can be a deletion, addition, or substitution of a nucleotide or nucleotides in the gene, which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

As used herein, the term “gain-of-function disorder” refers to a disorder characterized by a gain-of-function mutation. In one embodiment, the gain-of-function disorder is a neurodegenerative disease caused by a gain-of-function mutation, e.g., polyglutamine disorders and/or trinucleotide repeat diseases, for example, Huntington's disease. In another embodiment, the gain-of-function disorder is caused by a gain-of-function in an oncogene, the mutated gene product being a gain-of-function mutant, e.g., cancers caused by a mutation in the ret oncogene (e.g., ret-1), for example, endocrine tumors, medullary thyroid tumors, parathyroid hormone tumors, multiple endocrine neoplasia type2, and the like. Additional exemplary gain-of-function disorders include, but are not limited to, Alzheimer's disease, amyotrophic lateral sclerosis (ALS), human immunodeficiency disorder (HIV), and slow channel congenital myasthenic syndrome (SCCMS).

The term “trinucleotide repeat diseases,” as used herein, refers to any disease or disorder characterized by an expanded trinucleotide repeat region located within a gene, the expanded trinucleotide repeat region being causative of the disease or disorder. Examples of trinucleotide repeat diseases include, but are not limited to, spino-cerebellar ataxia type 12 spino-cerebellar ataxia type 8, fragile X syndrome, fragile XE Mental Retardation, Friedreich's ataxia and myotonic dystrophy. Exemplary trinucleotide repeat diseases for treatment according to the present disclosure are those characterized or caused by an expanded trinucleotide repeat region at the 5′ end of the coding region of a gene, the gene encoding a mutant protein, which causes or is causative of the disease or disorder. Certain trinucleotide diseases, for example, fragile X syndrome, where the mutation is not associated with a coding region may not be suitable for treatment according to the methodologies of the present disclosure, as there is no suitable mRNA to be targeted by RNAi. By contrast, disease such as Friedreich's ataxia may be suitable for treatment according to the methodologies of this disclosure because, although the causative mutation is not within a coding region (i.e., lies within an intron), the mutation may be within, for example, an mRNA precursor (e.g., a pre-spliced mRNA precursor).

The term “polyglutamine disorder,” as used herein, refers to any disease or disorder characterized by an expanded of a (CAG), repeats at the 5′ end of the coding region (thus encoding an expanded polyglutamine region in the encoded protein). In one embodiment, polyglutamine disorders are characterized by a progressive degeneration of nerve cells. Examples of polyglutamine disorders include, but are not limited to, Huntington's disease, spino-cerebellar ataxia type 1, spino-cerebellar ataxia type 2, spino-cerebellar ataxia type 3 (also known as Machado-Joseph disease), and spino-cerebellar ataxia type 6, spino-cerebellar ataxia type 7, dentatoiubral-pallidoluysian atrophy and the like.

The term “single nucleotide polymorphism disorder” or “SNP disorder” refers to a disorder characterized by the presence of an SNP, e.g., a heterozygous SNP. SNP disorders include, but are not limited to, phenylketonuria, cystic fibrosis, sickle-cell anemia, albinism, Huntington's disease, myotonic dystrophy type 1, hypercholesterolemia (autosomal dominant, type B), neurofibromatosis (type 1), polycystic kidney disease (1 and 2), hemophilia A, Duchenne's muscular dystrophy, X-linked hypophosphatemic rickets, Rett's syndrome, non-obstructive spermatogenic failure and the like. An exemplary heterozygous SNP disorder is Huntington's disease.

In certain aspects, a double-stranded RNA (dsRNA) is provided comprising a first strand of about 15-35 nucleotides that is complementary to a region of a gene comprising an allelic polymorphism, and a second strand of about 15-35 nucleotides that is complementary to at least a portion of the first strand, wherein the first strand comprises a single nucleotide polymorphism (SNP) position nucleotide at a position 2 to 7 from the 5′ end that is complementary to the allelic polymorphism; and a mismatch (MM) position nucleotide located 2-11 nucleotide from the SNP position nucleotide that is a mismatch with a nucleotide in the gene. In exemplary embodiments, the SNP position nucleotide is at a position 2, 4 or 6 from the 5′ end and the mismatch (MM) position nucleotide is located 2-6 nucleotides from the SNP position nucleotide.

As used herein, a “single nucleotide polymorphism position nucleotide” or a “SNP position nucleotide” refers to the position of an RNA described herein (e.g., the first strand of a dsRNA) that corresponds to the polymorphic position of a target nucleic acid sequence (i.e., either the mutant nucleotide corresponding to the SNP allele or the wild-type nucleotide corresponding to the wild-type allele). For example, a strand may be labeled “SNP2,” “SNP3,” or “SNP4” to denote the position of the SNP as being 2, 3, or 4 nucleotides from the 5′ end of the strand.

In certain exemplary embodiments, a SNP position nucleotide is within a seed region. In certain exemplary embodiments, a SNP position nucleotide is located between position 2 and position 7 from the 5′ end, between position 2 and position 6 from the 5′ end, or between position 2 and position 5 from the 5′ end. In certain exemplary embodiments, a SNP position nucleotide is located at position 2 from the 5′ end, at position 3 from the 5′ end, at position 4 from the 5′ end, at position 5 from the 5′ end, at position 6 from the 5′ end, or at position 7 from the 5′ end of an RNA described herein (e.g., the first strand of a dsRNA). In certain exemplary embodiments, a SNP position nucleotide is located at a position set forth in Tables 5-7.

As used herein, the term “seed region” refers to a six-nucleotide stretch corresponding to positions 2-7 from the 5′ end of an RNA strand. siRNA recognition of the target mRNA is believed to be conferred by the seed region of its antisense strand.

As used herein, a “mismatch position nucleotide” or a “MM position nucleotide” refers to the position of an RNA described herein (e.g., the first strand of a dsRNA) that is in a position that does not correspond to the SNP position nucleotide. A MM position nucleotide can be defined by its position from the 5′ end or the 3′ end of an RNA described herein (e.g., the 5′ or the 3′ end of first strand of a dsRNA), or defined by its position relative to a SNP position nucleotide of an RNA described herein (e.g., a first strand of a dsRNA).

In certain exemplary embodiments, a MM position nucleotide is located 2-11 nucleotides, 2-10 nucleotides, 2-9 nucleotides, 2-8 nucleotides, 2-7 nucleotides, or 2-6 nucleotides from a SNP position nucleotide. In certain exemplary embodiments, a MM position nucleotide is located 11 nucleotides, 10 nucleotides, 9 nucleotides, 8 nucleotides, 7 nucleotides, 6 nucleotides, 5 nucleotides, 4 nucleotides, 3 nucleotides or 2 nucleotides from a SNP position nucleotide. In certain exemplary embodiments, a MM position nucleotide is located at a position set forth in Tables 5-7.

In one embodiment, an RNA described herein (e.g., the first strand of a dsRNA) is homologous to an allelic polymorphism except for one mismatched oligonucleotide at a particular position relative to the nucleotide corresponding to the allelic polymorphism. In certain embodiments, the mismatch is within about 6 nucleotides of the SNP position nucleotide, within about 5 nucleotides of the SNP position nucleotide, within about 4 nucleotides of the SNP position nucleotide, within about 3 nucleotide of the SNP position nucleotide, within about 2 nucleotide of the SNP position nucleotide, or within about 1 nucleotide of the SNP position nucleotide. In particular embodiments, the mismatch is not adjacent to a SNP position nucleotide.

In another embodiment, a SNP position nucleotide is at position 2, 3, 4, 5, or 6 from the 5′ end. In an embodiment, a SNP position nucleotide is at position 2 from the 5′ end. In an embodiment, is at position 3 from the 5′ end. In an embodiment, a SNP position nucleotide is at position 4 from the 5′ end. In an embodiment, a SNP position nucleotide is at position 5 from the 5′ end. In an embodiment, a SNP position nucleotide is at position 6 from the 5′ end.

In certain exemplary embodiments, an RNA described herein (e.g., the first strand of a dsRNA) comprises a MM position nucleotide at position 5, 7, 8, 11, 14, 15 or 16 from the 5′ end. In certain exemplary embodiments, an RNA described herein (e.g., the first strand of a dsRNA) comprises a MM position nucleotide 1, 2, 3, 4, 5, 8, 9, 10 or 11 nucleotides from the SNP position nucleotide.

In certain exemplary embodiments, an RNA described herein (e.g., the first strand of a dsRNA) comprises a SNP position nucleotide (referenced from the 5′ end)-MM position nucleotide (referenced from the 5′ end) combination selected from the group consisting of 2-7, 4-7, 4-8, 4-15, 6-5, 6-8, 6-11, 6-14, 6-16, 3-5, 3-7 and 3-8.

In a particularly exemplary embodiment, an RNA described herein (e.g., the first strand of a dsRNA) comprises an SNP position nucleotide at position 6 from the 5′ end and a MM position nucleotide at position 11 from the 5′ end. In another particularly exemplary embodiment, an RNA described herein (e.g., the first strand of a dsRNA) comprises an SNP position nucleotide at position 4 from the 5′ end and a mismatch at position 7 from the 5′ end.

In one aspect, the double-stranded RNAs provided herein selectively silence a mutant allele having an allelic polymorphism. In an embodiment, the double-stranded RNAs provided herein silence a mutant allele having an allelic polymorphism and do not affect the wild-type allele of the same gene. In another embodiment, the double-stranded RNAs provided herein silence a mutant allele having an allelic polymorphism and silence the wild-type allele of the same gene to a lesser extent than the mutant allele.

Accordingly, in one aspect, the present disclosure provides a method of treating a subject having or at risk of having a disease characterized or caused by a mutant protein associated with an allelic polymorphism by administering to the subject an effective amount of an RNAi agent targeting an allelic polymorphism within a gene encoding a mutant protein (e.g., huntingtin protein), such that sequence-specific interference of a gene occurs resulting in an effective treatment for the disease.

In one aspect, RNA silencing agents disclosed herein preferentially silence a mutant allele comprising a polymorphism more efficiently than the corresponding wild-type allele. In certain exemplary embodiments, dsRNAs disclosed herein silence the allele comprising a polymorphism about 20%, about 30%, about 40%, about 50%, about 60%, about 70%, about 80%, or about 90% more than the corresponding wild-type allele. In an embodiment, RNA silencing agents disclosed herein silence the allele comprising a polymorphism at least about 50% more than the corresponding wild-type allele. In certain exemplary embodiments, dsRNAs disclosed herein silence the allele comprising a polymorphism at least about 5 times, about 10 times, about 15 times, about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, about 100 times, about 110 times, about 120 times, about 130 times, about 140 times, about 150 times, about 160 times, about 170 times, about 180 times, about 190 times, about 200 times, about 250 times, about 300 times, about 350 times, about 400 times, about 450 times, or up to about 500 times the level of silencing of the corresponding wild-type allele.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “antisense oligonucleotide” or “ASO” refers to a nucleic acid (e.g., an RNA), having sufficient sequence complementarity to a target an RNA (e.g., a SNP-containing mRNA or a SNP-containing pre-mRNA) in order to block a region of a target RNA in an effective manner, e.g., in a manner effective to inhibit translation of a target mRNA and/or splicing of a target pre-mRNA. An antisense oligonucleotide having a “sequence sufficiently complementary to a target RNA” means that the antisense agent has a sequence sufficient to mask a binding site for a protein that would otherwise modulate splicing and/or that the antisense agent has a sequence sufficient to mask a binding site for a ribosome and/or that the antisense agent has a sequence sufficient to alter the three-dimensional structure of the targeted RNA to prevent splicing and/or translation.

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, Van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

Linkers useful in conjugated compounds of the disclosure include glycol chains (e.g., polyethylene glycol), alkyl chains, peptides, RNA, DNA, and combinations thereof. As used herein, the abbreviation “TEG” refers to triethylene glycol.

### Design of Oligonucleotides

In certain embodiments, an oligonucleotide, e.g., an siRNA, of the disclosure is a duplex containing a sense strand and complementary antisense strand, the antisense strand having sufficient complementary to a target mRNA containing an allelic polymorphism to mediate RNAi. In exemplary embodiments, the siRNA molecule has a length from about 10-50 or more nucleotides, i.e., each strand comprises 10-50 nucleotides (or nucleotide analogs). In particularly exemplary embodiments, the siRNA molecule has a length from about 15-35, e.g., about 15, about 16, about 17, about 18, about 19, about 20, about 21, about 22, about 23, about 24, about 25, about 26, about 27, about 28, about 29, about 30, about 31, about 32, about 33, about 34 or about 35 nucleotides in each strand, wherein one of the strands is sufficiently complementary to a target region.

In exemplary embodiments, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. In exemplary embodiments, the siRNA molecule has a length from about 10-50 or more nucleotides, i.e., each strand comprises 10-50 nucleotides (or nucleotide analogs). In particularly exemplary embodiments, the siRNA molecule has a length from about 15-35, e.g., about 15, about 16, about 17, about 18, about 19, about 20, about 21, about 22, about 23, about 24, about 25, about 26, about 27, about 28, about 29, about 30, about 31, about 32, about 33, about 34 or about 35 nucleotides in each strand, wherein one of the strands is substantially complementary to a target sequence containing an allelic polymorphism, and the other strand is complementary or substantially complementary to the first strand. In an embodiment, the siRNA molecule in fully complementary to a target sequence containing an allelic polymorphism except for one additional mismatch, also known as secondary mismatch.

In some embodiments, each strand contains from 10 to 50 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, 45 nucleotides or nucleotide analogs, 46 nucleotides or nucleotide analogs, 47 nucleotides or nucleotide analogs, 48 nucleotides or nucleotide analogs, 49 nucleotides or nucleotide analogs, or 50 nucleotides or nucleotide analogs). In some embodiments, each strand contains from 10 to 49 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, 45 nucleotides or nucleotide analogs, 46 nucleotides or nucleotide analogs, 47 nucleotides or nucleotide analogs, 48 nucleotides or nucleotide analogs, or 49 nucleotides or nucleotide analogs). In some embodiments, each strand contains from 10 to 48 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, 45 nucleotides or nucleotide analogs, 46 nucleotides or nucleotide analogs, 47 nucleotides or nucleotide analogs, or 48 nucleotides or nucleotide analogs). In some embodiments, each strand contains from 10 to 47 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, 45 nucleotides or nucleotide analogs, 46 nucleotides or nucleotide analogs, or 47 nucleotides or nucleotide analogs). In some embodiments, each strand contains from 10 to 46 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, 45 nucleotides or nucleotide analogs, or 46 nucleotides or nucleotide analogs). In some embodiments, each strand contains from 10 to 45 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, 44 nucleotides or nucleotide analogs, or 45 nucleotides or nucleotide analogs). In some embodiments, each strand contains from 10 to 44 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, 43 nucleotides or nucleotide analogs, or 44 nucleotides or nucleotide analogs). In some embodiments, each strand contains from 10 to 43 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, 42 nucleotides or nucleotide analogs, or 43 nucleotides or nucleotide analogs). In some embodiments, each strand contains from 10 to 42 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, 41 nucleotides or nucleotide analogs, or 42 nucleotides or nucleotide analogs). In some embodiments, each strand contains from 10 to 41 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, 40 nucleotides or nucleotide analogs, or 41 nucleotides or nucleotide analogs). In some embodiments, each strand contains from 10 to 40 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, 39 nucleotides or nucleotide analogs, or 40 nucleotides or nucleotide analogs). In some embodiments, each strand contains from 10 to 39 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, 38 nucleotides or nucleotide analogs, or 39 nucleotides or nucleotide analogs). In some embodiments, each strand contains from 10 to 38 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, 37 nucleotides or nucleotide analogs, or 38 nucleotides or nucleotide analogs). In some embodiments, each strand contains from 10 to 37 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, 36 nucleotides or nucleotide analogs, or 37 nucleotides or nucleotide analogs). In some embodiments, each strand contains from 10 to 36 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, 35 nucleotides or nucleotide analogs, or 36 nucleotides or nucleotide analogs). In some embodiments, each strand contains from 10 to 35 nucleotides or nucleotide analogs (e.g., 10 nucleotides or nucleotide analogs, 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs). In some embodiments, each strand contains from 11 to 35 nucleotides or nucleotide analogs (e.g., 11 nucleotides or nucleotide analogs, 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs). In some embodiments, each strand contains from 12 to 35 nucleotides or nucleotide analogs (e.g., 12 nucleotides or nucleotide analogs, 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs). In some embodiments, each strand contains from 13 to 35 nucleotides or nucleotide analogs (e.g., 13 nucleotides or nucleotide analogs, 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs). In some embodiments, each strand contains from 14 to 35 nucleotides or nucleotide analogs (e.g., 14 nucleotides or nucleotide analogs, 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs). In some embodiments, each strand contains from 15 to 35 nucleotides or nucleotide analogs (e.g., 15 nucleotides or nucleotide analogs, 16 nucleotides or nucleotide analogs, 17 nucleotides or nucleotide analogs, 18 nucleotides or nucleotide analogs, 19 nucleotides or nucleotide analogs, 20 nucleotides or nucleotide analogs, 21 nucleotides or nucleotide analogs, 22 nucleotides or nucleotide analogs, 23 nucleotides or nucleotide analogs, 24 nucleotides or nucleotide analogs, 25 nucleotides or nucleotide analogs, 26 nucleotides or nucleotide analogs, 27 nucleotides or nucleotide analogs, 28 nucleotides or nucleotide analogs, 29 nucleotides or nucleotide analogs, 30 nucleotides or nucleotide analogs, 31 nucleotides or nucleotide analogs, 32 nucleotides or nucleotide analogs, 33 nucleotides or nucleotide analogs, 34 nucleotides or nucleotide analogs, or 35 nucleotides or nucleotide analogs).

In some embodiments, the siRNA contains a sense strand and an antisense strand (e.g., as described above), and the antisense strand has a length of from 15 to 25 nucleotides or nucleotide analogs, such as a length of from 16 to 24 nucleotides or nucleotide analogs, a length of from 17 to 23 nucleotides or nucleotide analogs, a length of from 18 to 22 nucleotides or nucleotide analogs, or a length of 19 to 21 nucleotides or nucleotide analogs. In some embodiments, the antisense strand has a length of 15 nucleotides or nucleotide analogs. In some embodiments, the antisense strand has a length of 16 nucleotides or nucleotide analogs. In some embodiments, the antisense strand has a length of 17 nucleotides or nucleotide analogs. In some embodiments, the antisense strand has a length of 18 nucleotides or nucleotide analogs. In some embodiments, the antisense strand has a length of 19 nucleotides or nucleotide analogs. In some embodiments, the antisense strand has a length of 20 nucleotides or nucleotide analogs. In some embodiments, the antisense strand has a length of 21 nucleotides or nucleotide analogs. In some embodiments, the antisense strand has a length of 22 nucleotides or nucleotide analogs. In some embodiments, the antisense strand has a length of 23 nucleotides or nucleotide analogs. In some embodiments, the antisense strand has a length of 24 nucleotides or nucleotide analogs. In some embodiments, the antisense strand has a length of 25 nucleotides or nucleotide analogs.

In some embodiments, the siRNA contains a sense strand and an antisense strand (e.g., as described above), and the sense strand has a length of from 10 to 20 nucleotides or nucleotide analogs, such as a length of from 11 to 19 nucleotides or nucleotide analogs, a length of from 12 to 18 nucleotides or nucleotide analogs, a length of from 13 to 17 nucleotides or nucleotide analogs, or a length of from 14 to 16 nucleotides or nucleotide analogs. In some embodiments, the sense strand has a length of 10 nucleotides or nucleotide analogs. In some embodiments, the sense strand has a length of 11 nucleotides or nucleotide analogs. In some embodiments, the sense strand has a length of 12 nucleotides or nucleotide analogs. In some embodiments, the sense strand has a length of 13 nucleotides or nucleotide analogs. In some embodiments, the sense strand has a length of 14 nucleotides or nucleotide analogs. In some embodiments, the sense strand has a length of 15 nucleotides or nucleotide analogs. In some embodiments, the sense strand has a length of 16 nucleotides or nucleotide analogs. In some embodiments, the sense strand has a length of 17 nucleotides or nucleotide analogs. In some embodiments, the sense strand has a length of 18 nucleotides or nucleotide analogs. In some embodiments, the sense strand has a length of 19 nucleotides or nucleotide analogs. In some embodiments, the sense strand has a length of 20 nucleotides or nucleotide analogs.

In some embodiments, the siRNA contains a sense strand and an antisense strand (e.g., as described above), wherein the antisense strand has a length of from 15 to 25 nucleotides or nucleotide analogs and the sense strand has a length of from 10 to 20 nucleotides or nucleotide analogs. In some embodiments, the antisense strand has a length of from 16 to 24 nucleotides or nucleotide analogs and the sense strand has a length of from 11 to 19 nucleotides or nucleotide analogs. In some embodiments, the antisense strand has a length of from 17 to 23 nucleotides or nucleotide analogs and the sense strand has a length of from 12 to 18 nucleotides or nucleotide analogs. In some embodiments, the antisense strand has a length of from 18 to 22 nucleotides or nucleotide analogs and the sense strand has a length of from 13 to 17 nucleotides or nucleotide analogs. In some embodiments, the antisense strand has a length of from 19 to 21 nucleotides or nucleotide analogs and the sense strand has a length of from 14 to 16 nucleotides or nucleotide analogs.

In some embodiments, the siRNA contains a sense strand and an antisense strand (e.g., as described above), wherein the antisense strand has a length of 20 nucleotides or nucleotide analogs and the sense strand has a length of 15 nucleotides or nucleotide analogs.

In some embodiments, the siRNA contains a sense strand and an antisense strand (e.g., as described above), wherein the antisense strand has a length of 21 nucleotides or nucleotide analogs and the sense strand has a length of 15 nucleotides or nucleotide analogs.

In some embodiments, the siRNA contains a sense strand and an antisense strand (e.g., as described above), wherein the antisense strand has a length of 20 nucleotides or nucleotide analogs and the sense strand has a length of 16 nucleotides or nucleotide analogs.

In some embodiments, the siRNA contains a sense strand and an antisense strand (e.g., as described above), wherein the antisense strand has a length of 21 nucleotides or nucleotide analogs and the sense strand has a length of 16 nucleotides or nucleotide analogs.

Generally, siRNAs can be designed by using any method known in the art, for instance, by using the following protocol:

1. The siRNA may be specific for a target sequence which contains an allelic polymorphism. In exemplary embodiments, the first strand is substantially complementary to the target sequence containing an allelic polymorphism having one mismatch to the target sequence containing an allelic polymorphism, and the other strand is substantially complementary to the first strand. In an embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus, in one embodiment, the disclosure includes nucleic acid molecules having 35-55% G/C content.

2. The sense strand of the siRNA is designed based on the sequence of the selected target site and the position of the allelic polymorphism. In exemplary embodiments, the RNA silencing agents of the disclosure do not elicit a PKR response (i.e., are of a sufficiently short length). However, longer RNA silencing agents may be useful, for example, in cell types incapable of generating a PKR response or in situations where the PKR response has been down-regulated or dampened by alternative means.

The siRNA molecules of the disclosure have sufficient complementarity with the target sequence such that the siRNA can mediate RNAi. In general, siRNA containing nucleotide sequences sufficiently identical to a target sequence portion of the target gene to effect RISC-mediated cleavage of the target gene are used. Accordingly, in an exemplary embodiment, the sense strand of the siRNA is designed have to have a sequence sufficiently identical to a portion of the target which contains an allelic polymorphism. The disclosure has the advantage of being able to tolerate certain sequence variations to enhance efficiency and specificity of RNAi. In an aspect, the sense strand has 1 mismatched nucleotide with a target region containing an allelic polymorphism, such as a target region that differs by at least one base pair between a wild-type and mutant allele, e.g., a target region comprising the gain-of-function mutation, and the other strand is identical or substantially identical to the first strand. In some embodiments, the mismatch is 4 nucleotides upstream, 3 nucleotides upstream nucleotide corresponding to the allelic polymorphism, 2 nucleotides upstream nucleotide corresponding to the allelic polymorphism, 1 nucleotide upstream, 1 nucleotide downstream nucleotide corresponding to the allelic polymorphism, 2 nucleotides downstream nucleotide corresponding to the allelic polymorphism, 3 nucleotides downstream nucleotide corresponding to the allelic polymorphism, 4 nucleotides downstream nucleotide corresponding to the allelic polymorphism, or 5 nucleotides downstream nucleotide corresponding to the allelic polymorphism. In some embodiments, the mismatch is not adjacent to the nucleotide corresponding to the allelic polymorphism. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions x 100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). An exemplary, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17): 3389-3402. In another embodiment, the alignment is optimized by introducing appropriate gaps and percent identity is determined over the entire length of the sequences aligned (i.e., a global alignment). An exemplary, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant: wild-type mismatch is a purine: purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet the criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional exemplary hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2 (# of A+T bases)+4 (# of G+C bases). For hybrids between 18 and 49 base pairs in length, Tm(° C.)=81.5+16.6 (log 10 [Na+])+0.41 (% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference in its entirety for all purposes.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

Sites of siRNA-mRNA complementation are selected, which result in optimal mRNA specificity and maximal mRNA cleavage.

siRNA-Like Molecules

siRNA-like molecules of the present disclosure have a sequence (i.e., have a strand having a sequence) that is “sufficiently complementary” to a target sequence of an mRNA (e.g. an htt mRNA) to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected, such that it forms a wobble base pair (e.g., G:U) or a mismatched base pair (G: A, C: A, C:U, G: G, A: A, C:C, U:U). In a further exemplary embodiment, the “bulge” is centered at nucleotide positions 12 and 13 from the 5′ end of the miRNA molecule.

### Modified RNA Silencing Agents

In certain aspects of the disclosure, an RNA silencing agent (or any portion thereof) of the disclosure as described supra may be modified such that the activity of the agent is further improved. For example, the RNA silencing agents described in above may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the disclosure may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA).

In certain exemplary embodiments, the RNA silencing agents of the disclosure are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is typically utilized because it has relatively minor effect on the stability of the RNA duplex or the duplex formed by the guide strand of the RNA silencing agent and the target mRNA. Exemplary universal nucleotide include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly exemplary embodiments, the universal nucleotide is an inosine residue or a naturally occurring analog thereof.

In certain embodiments, the RNA silencing agents of the disclosure are modified by the introduction of at least one destabilizing nucleotide within 11 nucleotides from a specificity-determining nucleotide (e.g., within 11 nucleotides from the nucleotide which recognizes the disease-related polymorphism (e.g., a SNP position nucleotide)). For example, the destabilizing nucleotide may be introduced at a position that is within 11, 10, 9, 8, 7, 6, 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In particular exemplary embodiments, the destabilizing nucleotide is introduced in the same strand or strand portion that contains the specificity-determining nucleotide.

In certain embodiments, the RNA silencing agents of the disclosure are modified by the introduction of a modified intersubunit linkage of Formula 1:

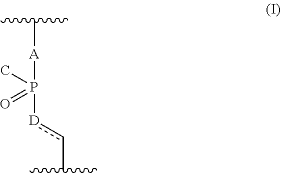

wherein:
D is selected from the group consisting of O, OCH₂, OCH, CH₂, and CH;
C is selected from the group consisting of O⁻, OH, OR¹, NH⁻, NH₂, S⁻, and SH;
A is selected from the group consisting of O and CH₂;
R¹ is a protecting group;
[symbol] is an optional double bond; and
the intersubunit is bridging two optionally modified nucleosides.

In an embodiment, when C is O⁻, either A or D is not O.

In an embodiment, D is CH₂. In another embodiment, the modified intersubunit linkage of Formula VIII is a modified intersubunit linkage of Formula 2:

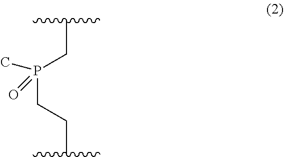 [SMILES: *CCP(C)(=O)C*]

In an embodiment, D is O. In another embodiment, the modified intersubunit linkage of Formula VIII is a modified intersubunit linkage of Formula 3:

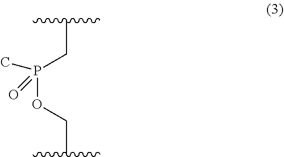 [SMILES: *COP(C)(=O)C*]

In an embodiment, D is CH. In another embodiment, the modified intersubunit linkage of Formula VIII is a modified intersubunit linkage of Formula 4:

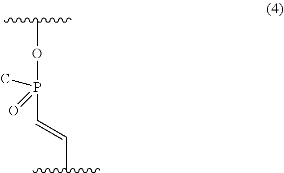 [SMILES: */C=C/P(C)(=O)O*]

In another embodiment, the modified intersubunit linkage is a modified intersubunit linkage of Formula 5:

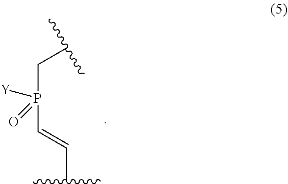

In an embodiment, D is OCH₂. In another embodiment, the modified intersubunit linkage is a modified intersubunit linkage of Formula 6:

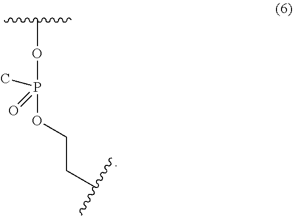 [SMILES: *CCOP(C)(=O)O*]

In another embodiment, the modified intersubunit linkage of Formula VII is a modified intersubunit linkage of Formula 7:

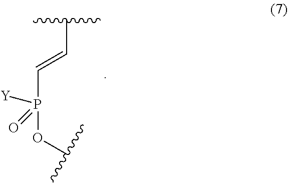

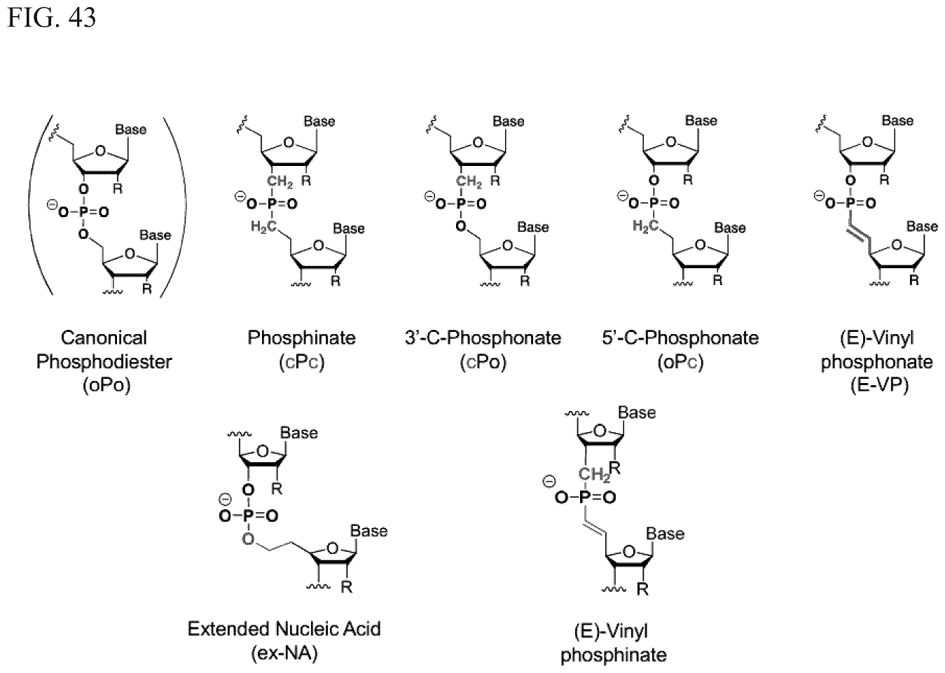

In certain embodiments, the RNA silencing agents of the disclosure are modified by the introduction of one or more of the intersubunit linkers of FIG. 43. In an exemplary embodiment, an intersubunit linker of FIG. 43 is inserted between the SNP position nucleotide and a nucleotide at a position directly adjacent to and on either side of the SNP position nucleotide of the antisense strand.

In certain embodiments, the RNA silencing agents of the disclosure are modified by the introduction of one or more vinyl phosphonate (VP) motifs in the intersubunit linker having the following formula:

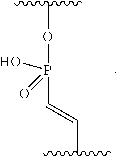 [SMILES: */C=C/P(=O)(O)O*]

In certain embodiments, a VP motif is inserted at any position(s) of an oligonucleotide, e.g., an RNA. For example, for an oligonucleotide having a length of 20 nucleotides, a VP motif can be inserted at position 1-2, 2-3, 3-4, 4-5, 5-6, 6-7, 7-8, 8-9, 9-10, 10-11, 11-12, 12-13, 13-14, 14-15, 15-16, 16-17, 17-18, 18-19 or 19-20 and at any combinations of these.

In certain exemplary embodiments, a VP motif is inserted at one or more of positions 1-2, 5-6, 6-7, 10-11, 18-19 and/or 19-20 of the antisense strand.

In other exemplary embodiments, a VP motif is inserted at one or more of positions 1-2, 6-7, 10-11 and/or 19-20 of the antisense strand.

In an exemplary embodiment, a VP motif is inserted next to (i.e., between a SNP position nucleotide and a nucleotide at a position directly adjacent to and on either side of) the SNP position nucleotide of the antisense strand. In another exemplary embodiment, a VP motif is inserted next to (i.e., between a MM position nucleotide and a nucleotide at a position directly adjacent to and on either side of) the MM position nucleotide of the antisense strand.

### 2) Modifications to Enhance Efficacy and Specificity

In certain embodiments, the RNA silencing agents of the disclosure may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the disclosure or an siRNA produced from a shRNA) into RISC in favor of the sense strand, such that the antisense strand preferentially guides cleavage or translational repression of a target mRNA, and thus increasing or improving the efficiency of target cleavage and silencing. In exemplary embodiments, the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the disclosure may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the disclosure may be enhanced such that there is at least one mismatched base pair between the 5′ end of the first or antisense strand and the 3′ end of the sense strand portion. In an exemplary embodiment, the mismatched base pair is selected from the group consisting of G: A, C: A, C:U, G: G, A: A, C:C and U:U. In another embodiment, the asymmetry of an RNA silencing agent of the disclosure may be enhanced such that there is at least one wobble base pair, e.g., G:U, between the 5′ end of the first or antisense strand and the 3′ end of the sense strand portion. In another embodiment, the asymmetry of an RNA silencing agent of the disclosure may be enhanced such that there is at least one base pair comprising a rare nucleotide, e.g., inosine (I). In exemplary embodiments, the base pair is selected from the group consisting of an I: A, I:U and I:C. In yet another embodiment, the asymmetry of an RNA silencing agent of the disclosure may be enhanced such that there is at least one base pair comprising a modified nucleotide. In particular embodiments, the modified nucleotide is selected from the group consisting of 2-amino-G, 2-amino-A, 2,6-diamino-G, and 2,6-diamino-A.

In certain embodiments, the RNA silencing agents of the disclosure are altered at one or more intersubunit linkages in an oligonucleotide by the introduction of a vinyl phosphonate (VP) motif having the following formula:

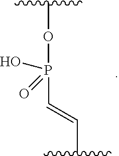 [SMILES: */C=C/P(=O)(O)O*]

3) RNA Silencing Agents with Enhanced Stability

The RNA silencing agents of the present disclosure can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a particular aspect, the disclosure features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a particular embodiment of the present disclosure, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Particularly, the ends may be stabilized by incorporating modified nucleotide analogues.

In certain embodiments, the RNA silencing agents of the disclosure are altered at one or more intersubunit linkages in an oligonucleotide by the introduction of a vinyl phosphonate (VP) motif having the following formula:

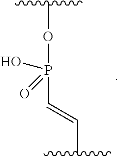 [SMILES: */C=C/P(=O)(O)O*]

A variety of oligonucleotide types (e.g., gapmers, mixmers, miRNA inhibitors, splice-switching oligonucleotides (“SSOs”), phosphorodiamidate morpholino oligonucleotides (“PMOs”), peptide nucleic acids (“PNAs”) and the like) can be used in the oligonucleotides described herein, optionally utilizing various combinations of modifications (e.g., chemical modifications) and/or conjugations described herein and in, e.g., U.S. Ser. No. 15/089,423; U.S. Ser. No. 15/236,051; U.S. Ser. No. 15/419,593; U.S. Ser. No. 15/697,120 and U.S. Pat. No. 9,809,817; and U.S. Ser. No. 15/814,350 and U.S. Pat. No. 9,862,350, each of which is incorporated herein by reference in its entirety for all purposes.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly exemplary modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moieties of the instant disclosure. Additional modified residues include, deoxy-abasic, inosine, N3-methyl-uridine, N6,N6-dimethyl-adenosine, pseudouridine, purine ribonucleoside and ribavirin. In a particularly exemplary embodiment, the 2′ moiety is a methyl group such that the linking moiety is a 2′-O-methyl oligonucleotide.

In an exemplary embodiment, the RNA silencing agent of the disclosure comprises locked nucleic acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol. 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the disclosure comprises peptide nucleic acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254:1497-1500).

In certain exemplary embodiments, nucleobase-modified ribonucleotides, i.e., ribonucleotides, containing at least one non-naturally occurring nucleobase instead of a naturally occurring nucleobase, are used. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the disclosure includes RNA silencing agents having two complementary strands of nucleic acid, wherein the two strands are crosslinked. The disclosure also includes RNA silencing agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′-OMe moiety on a U in a sense or antisense strand, but especially on a sense strand, or provision of a 2′-OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (in most cases, sequence changes are located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′-OMe moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### 4) Modifications to Enhance Cellular Uptake

In other embodiments, RNA silencing agents may be modified with chemical moieties, for example, to enhance cellular uptake by target cells (e.g., neuronal cells). Thus, the disclosure includes RNA silencing agents which are conjugated or unconjugated (e.g., at its 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3): 137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2): 404-10 (1995) (describes nucleic acids linked to nanoparticles).

In a particular embodiment, a modification to the RNA silencing agents of the disclosure comprise a vinyl phosphonate (VP) motif in one or more intersubunit linkers of an oligonucleotide, wherein the VP motif has the following formula:

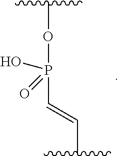 [SMILES: */C=C/P(=O)(O)O*]

In a particular embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl) cholenic acid, dimethoxytrityl, or phenoxazine.

### 5) Tethered Ligands

Other entities can be tethered to an RNA silencing agent of the disclosure. For example, a ligand tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. In certain exemplary embodiments, these are located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, typically covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of an RNA silencing agent into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolicd) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O (octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon.

In one aspect, the ligand is a lipid or lipid-based molecule. Such a lipid or lipid-based molecule typically binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. In a particular embodiment, the lipid based ligand binds HSA. However, it is desired that the affinity not be so strong that the HSA-ligand binding cannot be reversed. In another exemplary embodiment, the lipid based ligand binds HSA weakly or not at all.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, typically a helical cell-permeation agent. In certain exemplary embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an alpha-helical agent, which typically has a lipophilic face and a lipophobic face.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

### 6) Hydrophobic Moieties

In certain embodiments of the double-stranded RNAs provided herein, the RNA molecule is conjugated to one or more hydrophobic moieties (see PCT Pub. No. WO 2018/031933, which is incorporated herein by reference). In an embodiment, the hydrophobic moiety has an affinity for low density lipoprotein and/or intermediate density lipoprotein. In a related embodiment, the hydrophobic moiety is a saturated or unsaturated moiety having fewer than three double bonds.

In another embodiment, the hydrophobic moiety has an affinity for high density lipoprotein. In a related embodiment, the hydrophobic moiety is a polyunsaturated moiety having at three or more double bonds (e.g., having three, four, five, six, seven, eight, nine or ten double bonds). In a particular embodiment, the hydrophobic moiety is a polyunsaturated moiety having three double bonds. In a particular embodiment, the hydrophobic moiety is a polyunsaturated moiety having four double bonds. In a particular embodiment, the hydrophobic moiety is a polyunsaturated moiety having five double bonds. In a particular embodiment, the hydrophobic moiety is a polyunsaturated moiety having six double bonds.

In another embodiment, the hydrophobic moiety is selected from the group consisting of fatty acids, steroids, secosteroids, lipids, gangliosides and nucleoside analogs, and endocannabinoids.

In another embodiment, the hydrophobic moiety is a neuromodulatory lipid, e.g., an endocannabinoid. Non-limiting examples of endocannabinoids include: anandamide, arachidonoylethanolamine, 2-Arachidonyl glyceryl ether (noladin ether), 2-Arachidonyl glyceryl ether (noladin ether), 2-Arachidonoylglycerol, and N-Arachidonoyl dopamine.

In another embodiment, the hydrophobic moiety is an omega-3 fatty acid. Non-limiting examples of omega-3 fatty acids include, but are not limited to: hexadecatrienoic acid (HTA), alpha-linolenic acid (ALA), stearidonic acid (SDA), cicosatrienoic acid (ETE), eicosatetraenoic acid (ETA), eicosapentaenoic acid (EPA, Timnodonic acid), heneicosapentaenoic acid (HPA), docosapentaenoic acid (DPA, clupanodonic acid), docosahexaenoic acid (DHA, cervonic acid), tetracosapentaenoic acid, and tetracosahexaenoic acid (nisinic acid).

In another embodiment, the hydrophobic moiety is an omega-6 fatty acid. Non-limiting examples of omega-6 fatty acids include, but are not limited to: linoleic acid, gamma-linolenic acid (GLA), eicosadienoic acid, dihomo-gamma-linolenic acid (DGLA), arachidonic acid (AA), docosadienoic acid, adrenic acid, docosapentaenoic acid (osbond acid), tetracosatetraenoic acid, and tetracosapentaenoic acid.

In another embodiment, the hydrophobic moiety is an omega-9 fatty acid. Non-limiting examples of omega-9 fatty acids include, but are not limited to: oleic acid, eicosenoic acid, Mead acid, erucic acid, and nervonic acid.

In another embodiment, the hydrophobic moiety is a conjugated linolenic acid. Non-limiting examples of conjugated linolenic acids include, but are not limited to: α-calendic acid, β-calendic acid, jacaric acid, α-eleostearic acid, β-eleostearic acid, catalpic acid, and punicic acid.

In another embodiment, the hydrophobic moiety is a saturated fatty acid. Non-limiting examples of saturated fatty acids include, but are not limited to: caprylic acid, capric acid, docosanoic acid, lauric acid, myristic acid, palmitic acid, stearic acid, arachidic acid, behenic acid, lignoceric acid, and cerotic acid.

In another embodiment, the hydrophobic moiety is an acid selected from the group consisting of: rumelenic acid, α-parinaric acid, β-parinaric acid, bosseopentaenoic acid, pinolenic acid, and podocarpic acid.

In another embodiment, the hydrophobic moiety is selected from the group consisting of: docosanoic acid (DCA), docosahexaenoic acid (DHA), and eicosapentaenoic acid (EPA). In a particular embodiment, the hydrophobic moiety is docosanoic acid (DCA). In another particular embodiment, the hydrophobic moiety is DHA. In another particular embodiment, the hydrophobic moiety is EPA.

In another embodiment, the hydrophobic moiety is a secosteroid. In a particular embodiment, the hydrophobic moiety is calciferol. In another embodiment, the hydrophobic moiety is a steroid other than cholesterol.

In a particular embodiment, the hydrophobic moiety is not cholesterol.

In another embodiment, the hydrophobic moiety is an alkyl chain, a vitamin, a peptide, or a bioactive conjugate, including but not limited to: glycosphingolipids, polyunsaturated fatty acids, secosteroids, steroid hormones, or sterol lipids.

In an embodiment, a double-stranded RNA provided herein comprises one or more chemically-modified nucleotides. In a particular embodiment, the double-stranded RNA comprises alternating 2′-methoxy-nucleotides and 2′-fluoro-nucleotides. In another particular embodiment, one or more nucleotides of the double-stranded RNA are connected to adjacent nucleotides via phosphorothioate linkages. In certain embodiments of the dsRNAs disclosed herein, the mismatch nucleotide and the nucleotide(s) adjacent to the mismatch nucleotide are 2′-methoxy-ribonucleotides.

In another particular embodiment, the nucleotides at positions 1 and 2 from the 3′ end of the double-stranded RNAs provided herein are connected to adjacent nucleotides via phosphorothioate linkages. In yet another particular embodiment, the nucleotides at positions 1 and 2 from the 3′ end of the double-stranded RNAs and the nucleotides at positions 1 and 2 from the 5′ end of the double-stranded RNAs are connected to adjacent nucleotides via phosphorothioate linkages.

In one embodiment of a double-stranded RNA, the first oligonucleotide comprises at least 16 contiguous nucleotides, a 5′ end, a 3′ end, and has complementarity to a target, wherein:
(1) the first oligonucleotide comprises alternating 2′-methoxy-nucleotides and 2′-fluoro-nucleotides;
(2) the nucleotides at positions 2 and 14 from the 5′ end are not 2′-methoxy-nucleotides;
(3) the nucleotides are connected via phosphodiester or phosphorothioate linkages; and
(4) the nucleotides at positions 1-6 from the 3′ end, or positions 1-7 from the 3′ end, are connected to adjacent nucleotides via phosphorothioate linkages.

### 7) Advanced Stabilization Pattern

In one embodiment of the double-stranded RNAs provided herein:
(1) the first oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the first oligonucleotide are not 2′-methoxy-ribonucleotides;
(2) the second oligonucleotide comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the second oligonucleotide are 2′-methoxy-ribonucleotides;
(3) the nucleotides of the first oligonucleotide are connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and
(4) the nucleotides of the second oligonucleotide are connected to adjacent nucleotides via phosphodiester or phosphorothioate linkages, wherein the nucleotides at positions 1 and 2 from the 3′ end are connected to adjacent nucleotides via phosphorothioate linkages.

In one embodiment of the double-stranded RNAs, the first oligonucleotide has 3-7 more ribonucleotides than the second oligonucleotide.

In one embodiment, the double-stranded RNA comprises 11-16 base pair duplexes, wherein the nucleotides of each base pair duplex have different chemical modifications (e.g., one nucleotide has a 2′-fluoro modification and the other nucleotide has a 2′-methoxy).

In one embodiment of the double-stranded RNAs, the first oligonucleotide has 3-7 more ribonucleotides than the second oligonucleotide. In another embodiment.

In one embodiment, the first oligonucleotide is the antisense strand and the second oligonucleotide is the sense strand. See PCT Pub. No. WO 2016/161388, which is incorporated herein by reference.

In one embodiment, the first or second oligonucleotide comprises one or more VP intersubunit modifications having the following formula:

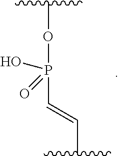 [SMILES: */C=C/P(=O)(O)O*]

### 8) Branched Oligonucleotides

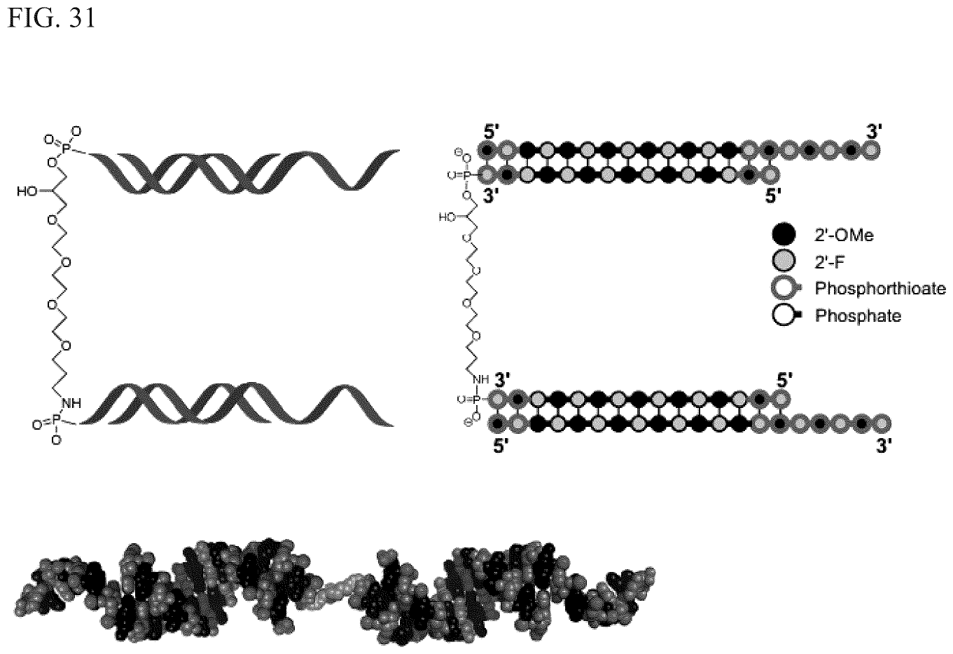

Two or more RNA silencing agents as disclosed above, for example oligonucleotide constructs such as siRNAs, may be connected to one another by one or more moieties independently selected from a linker, a spacer and a branching point, forming a branched oligonucleotide containing two or more RNA silencing agents. FIG. 31 illustrates an exemplary di-siRNA di-branched scaffolding for delivering two siRNAs. In representative embodiments, the nucleic acids of the branched oligonucleotide each comprise an antisense strand (or portions thereof), wherein the antisense strand has sufficient complementary to a heterozygous single nucleotide polymorphism to mediate an RNA-mediated silencing mechanism (e.g. RNAi). In other embodiments, there is provided a second type of branched oligonucleotides featuring nucleic acids that comprise a sense strand (or portions thereof) for silencing antisense transcripts, where the sense strand has sufficient complementarity to an antisense transcript to mediate an RNA-mediated silencing mechanism. In further embodiments, there is provided a third type of branched oligonucleotides including nucleic acids of both types, that is, a nucleic acid comprising an antisense strand (or portions thereof) and an oligonucleotide comprising a sense strand (or portions thereof).

In exemplary embodiments, the branched oligonucleotides may have two to eight RNA silencing agents attached through a linker. The linker may be hydrophobic. In a particular embodiment, branched oligonucleotides of the present application have two to three oligonucleotides. In one embodiment, the oligonucleotides independently have substantial chemical stabilization (e.g., at least 40% of the constituent bases are chemically-modified). In a particular embodiment, the oligonucleotides have full chemical stabilization (i.e., all of the constituent bases are chemically-modified). In some embodiments, branched oligonucleotides comprise one or more single-stranded phosphorothioated tails, each independently having two to twenty nucleotides. In a particular embodiment, each single-stranded tail has eight to ten nucleotides.

In certain embodiments, branched oligonucleotides are characterized by three properties: (1) a branched structure, (2) full metabolic stabilization, and (3) the presence of a single-stranded tail comprising phosphorothioate linkers. In a specific embodiment, branched oligonucleotides have 2 or 3 branches. It is believed that the increased overall size of the branched structures promotes increased uptake. Also, without being bound by a particular theory of activity, multiple adjacent branches (e.g., 2 or 3) are believed to allow each branch to act cooperatively and thus dramatically enhance rates of internalization, trafficking and release.

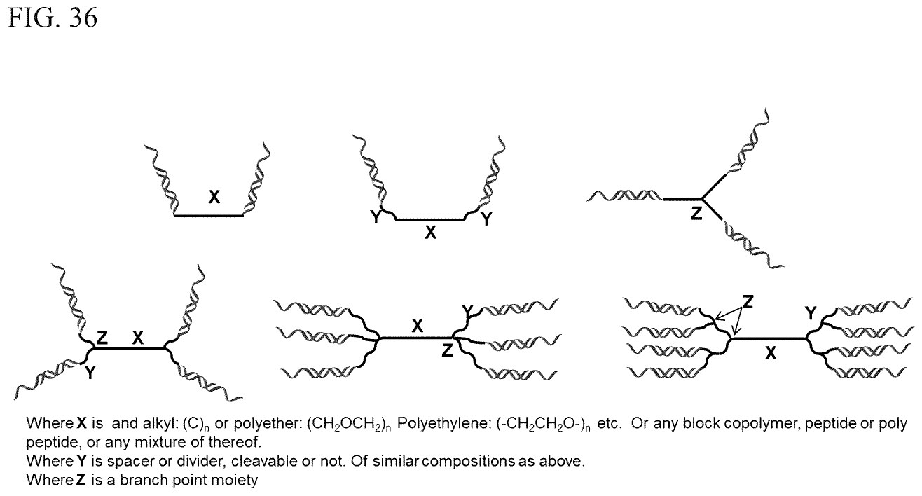

Branched oligonucleotides are provided in various structurally diverse embodiments. As shown in FIG. 36, for example, in some embodiments nucleic acids attached at the branching points are single stranded and consist of miRNA inhibitors, gapmers, mixmers, SSOs, PMOs, or PNAs. These single strands can be attached at their 3′ or 5′ end. Combinations of siRNA and single stranded oligonucleotides could also be used for dual function. In another embodiment, short nucleic acids complementary to the gapmers, mixmers, miRNA inhibitors, SSOs, PMOs, and PNAs are used to carry these active single-stranded nucleic acids and enhance distribution and cellular internalization. The short duplex region has a low melting temperature (Tₘ ˜37° C.) for fast dissociation upon internalization of the branched structure into the cell.

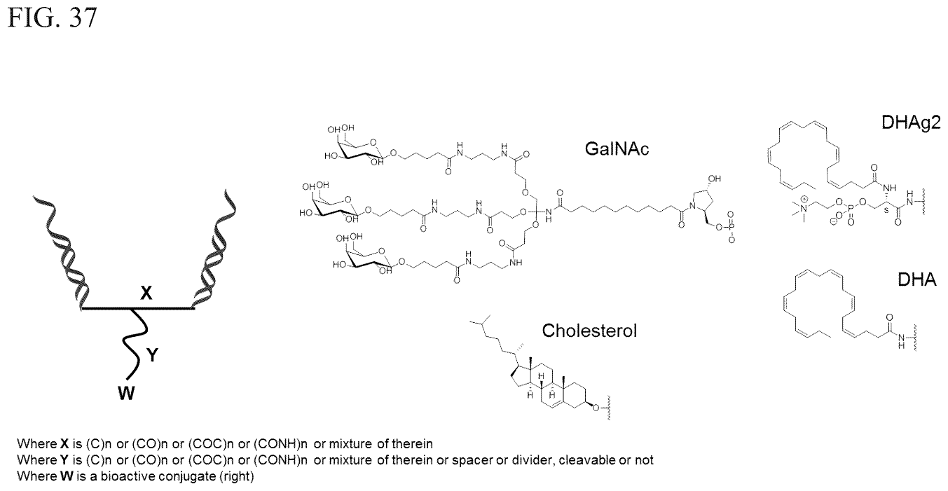

As shown in FIG. 37, Di-siRNA branched oligonucleotides may comprise chemically diverse conjugates. Conjugated bioactive ligands may be used to enhance cellular specificity and to promote membrane association, internalization, and serum protein binding. Examples of bioactive moieties to be used for conjugation include DHAg2, DHA, GalNAc, and cholesterol. These moieties can be attached to Di-siRNA either through the connecting linker or spacer, or added via an additional linker or spacer attached to another free siRNA end.

The presence of a branched structure improves the level of tissue retention in the brain more than 100-fold compared to non-branched compounds of identical chemical composition, suggesting a new mechanism of cellular retention and distribution. Branched oligonucleotides have unexpectedly uniform distribution throughout the spinal cord and brain. Moreover, branched oligonucleotides exhibit unexpectedly efficient systemic delivery to a variety of tissues, and very high levels of tissue accumulation.

Branched oligonucleotides comprise a variety of therapeutic nucleic acids, including ASOs, miRNAs, miRNA inhibitors, splice switching, PMOs, PNAs. In some embodiments, branched oligonucleotides further comprise conjugated hydrophobic moieties and exhibit unprecedented silencing and efficacy in vitro and in vivo.

### Linkers

In an embodiment of the branched oligonucleotide, each linker is independently selected from an ethylene glycol chain, an alkyl chain, a peptide, RNA, DNA, a phosphate, a phosphonate, a phosphoramidate, an ester, an amide, a triazole, and combinations thereof; wherein any carbon or oxygen atom of the linker is optionally replaced with a nitrogen atom, bears a hydroxyl substituent, or bears an oxo substituent. In one embodiment, each linker is an ethylene glycol chain. In another embodiment, each linker is an alkyl chain. In another embodiment, each linker is a peptide. In another embodiment, each linker is RNA. In another embodiment, each linker is DNA. In another embodiment, each linker is a phosphate. In another embodiment, each linker is a phosphonate. In another embodiment, each linker is a phosphoramidate. In another embodiment, each linker is an ester. In another embodiment, each linker is an amide. In another embodiment, each linker is a triazole. In another embodiment, each linker is a structure selected from the formulas of FIG. 37.

### 9) Compound of Formula (I)

In another aspect, provided herein is a branched oligonucleotide compound of formula (I):

L-(N)ₙ (I)

wherein L is selected from an ethylene glycol chain, an alkyl chain, a peptide, RNA, DNA, a phosphate, a phosphonate, a phosphoramidate, an ester, an amide, a triazole, and combinations thereof, wherein formula (I) optionally further comprises one or more branch point B, and one or more spacer S; wherein B is independently for each occurrence a polyvalent organic species or derivative thereof; S is independently for each occurrence selected from an ethylene glycol chain, an alkyl chain, a peptide, RNA, DNA, a phosphate, a phosphonate, a phosphoramidate, an ester, an amide, a triazole, and combinations thereof; N is an RNA duplex comprising a sense strand and an antisense strand, wherein the antisense strand comprises a region of complementarity which is substantially complementary to a region of a gene comprising an allelic polymorphism, wherein the antisense strand comprises: a single nucleotide polymorphism (SNP) position nucleotide at a position 2 to 7 from the 5′ end that is complementary to the allelic polymorphism; and a mismatch (MM) position nucleotide located 2-11 nucleotides from the SNP position nucleotide that is a mismatch with a nucleotide in the gene. In exemplary embodiments, the SNP position nucleotide is at a position 2, 4 or 6 from the 5′ end and the mismatch (MM) position nucleotide is located 2-6 nucleotides from the SNP position nucleotide.

The sense strand and antisense strand each independently comprise one or more chemical modifications; and n is 2, 3, 4, 5, 6, 7 or 8.

In an embodiment, the compound of formula (I) has a structure selected from formulas (I-1)-(I-9) of Table 1.

TABLE 1

| | |
|---|---|
| N—L—N | (I-1) |
| N—S—L—S—N | (I-2) |
| 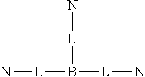 | (I-3) |
| 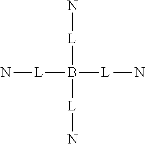 | (I-4) |
| 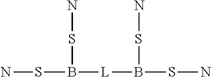 | (I-5) |
| 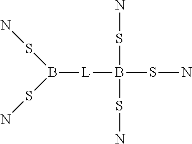 | (I-6) |
| 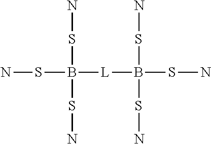 | (I-7) |
| 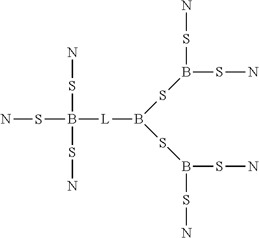 | (I-8) |
| 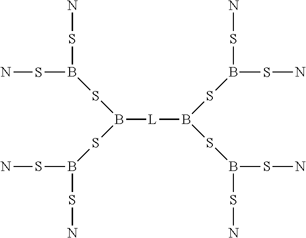 | (I-9) |

In one embodiment, the compound of formula (I) is formula (I-1). In another embodiment, the compound of formula (I) is formula (I-2). In another embodiment, the compound of formula (I) is formula (I-3). In another embodiment, the compound of formula (I) is formula (I-4). In another embodiment, the compound of formula (I) is formula (I-5). In another embodiment, the compound of formula (I) is formula (I-6). In another embodiment, the compound of formula (I) is formula (I-7). In another embodiment, the compound of formula (I) is formula (I-8). In another embodiment, the compound of formula (I) is formula (I-9).

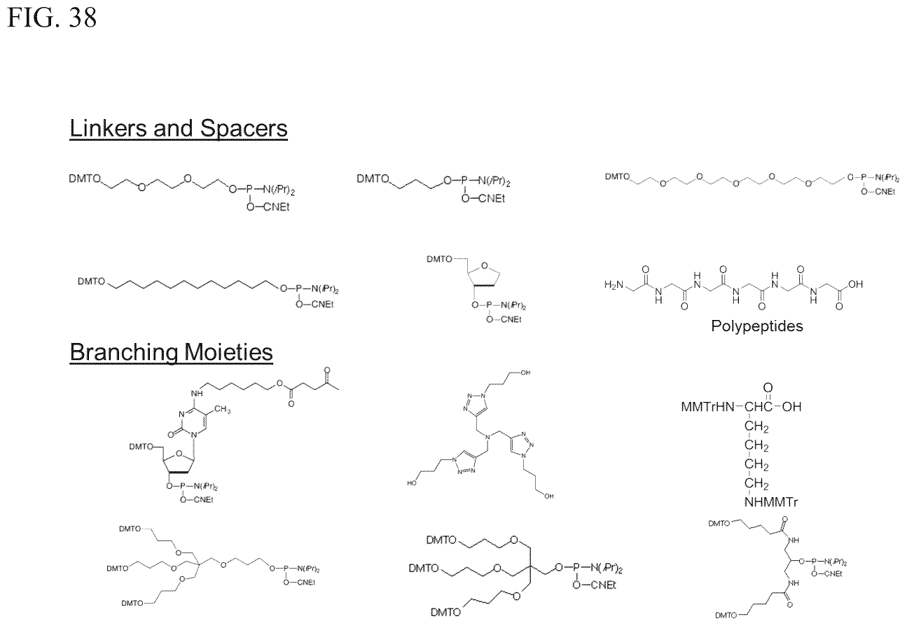

In an embodiment of the compound of formula (I), each linker is independently selected from an ethylene glycol chain, an alkyl chain, a peptide, RNA, DNA, a phosphate, a phosphonate, a phosphoramidate, an ester, an amide, a triazole, and combinations thereof; wherein any carbon or oxygen atom of the linker is optionally replaced with a nitrogen atom, bears a hydroxyl substituent, or bears an oxo substituent. In one embodiment of the compound of formula (I), each linker is an ethylene glycol chain. In another embodiment, each linker is an alkyl chain. In another embodiment of the compound of formula (I), each linker is a peptide. In another embodiment of the compound of formula (I), each linker is RNA. In another embodiment of the compound of formula (I), each linker is DNA. In another embodiment of the compound of formula (I), each linker is a phosphate. In another embodiment, each linker is a phosphonate. In another embodiment of the compound of formula (I), each linker is a phosphoramidate. In another embodiment of the compound of formula (I), each linker is an ester. In another embodiment of the compound of formula (I), each linker is an amide. In another embodiment of the compound of formula (I), each linker is a triazole. In another embodiment of the compound of formula (I), each linker is a structure selected from the formulas of FIG. 36 and FIG. 38.

In one embodiment of the compound of formula (I), B is a polyvalent organic species. In another embodiment of the compound of formula (I), B is a derivative of a polyvalent organic species. In one embodiment of the compound of formula (I), B is a triol or tetrol derivative. In another embodiment, B is a tri- or tetra-carboxylic acid derivative. In another embodiment, B is an amine derivative. In another embodiment, B is a tri- or tetra-amine derivative. In another embodiment, B is an amino acid derivative. In another embodiment of the compound of formula (I), B is selected from the formulas of FIG. 38.

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In an embodiment of the compound of formula (I), each nucleic acid comprises one or more chemically-modified nucleotides. In an embodiment of the compound of formula (I), each nucleic acid consists of chemically-modified nucleotides. In certain embodiments of the compound of formula (I), >95%, >90%, >85%, >80%, >75%, >70%, >65%, >60%, >55% or >50% of each nucleic acid comprises chemically-modified nucleotides.

In an embodiment, each antisense strand independently comprises a 5′ terminal group R selected from the groups of Table 2:

TABLE 2

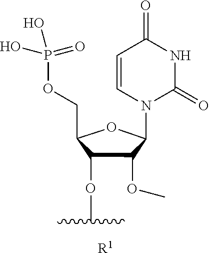

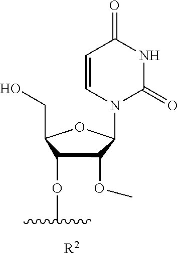

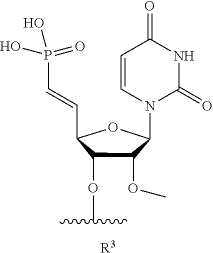

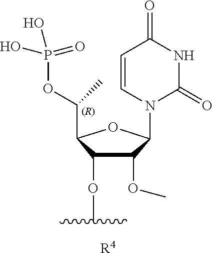

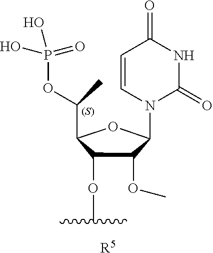

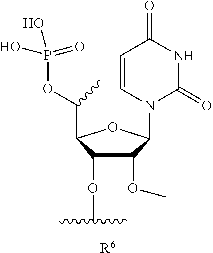

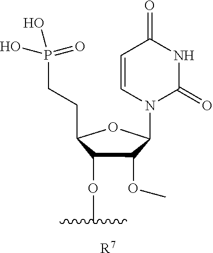

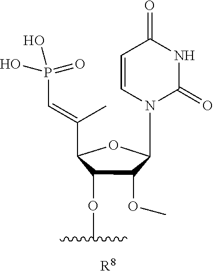

In one embodiment, R is R₁. In another embodiment, R is R₂. In another embodiment, R is R₃. In another embodiment, R is R₄. In another embodiment, R is R₅. In another embodiment, R is R₆. In another embodiment, R is R₇. In another embodiment, R is R₈.

### Structure of Formula (II)

In an embodiment, the compound of formula (I) the structure of formula (II):

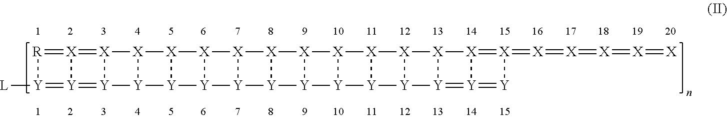

wherein X, for each occurrence, independently, is selected from adenosine, guanosine, uridine, cytidine, and chemically-modified derivatives thereof; Y, for each occurrence, independently, is selected from adenosine, guanosine, uridine, cytidine, and chemically-modified derivatives thereof; - represents a phosphodiester internucleoside linkage; ═ represents a phosphorothioate internucleoside linkage; and --- represents, individually for each occurrence, a base-pairing interaction or a mismatch.

In certain embodiments, the structure of formula (II) does not contain mismatches. In one embodiment, the structure of formula (II) contains 1 mismatch. In another embodiment, the compound of formula (II) contains 2 mismatches. In another embodiment, the compound of formula (II) contains 3 mismatches. In another embodiment, the compound of formula (II) contains 4 mismatches. In an embodiment, each nucleic acid consists of chemically-modified nucleotides.

In certain embodiments, >95%, >90%, >85%, >80%, >75%, >70%, >65%, >60%, >55% or >50% of X's of the structure of formula (II) are chemically-modified nucleotides. In other embodiments, >95%, >90%, >85%, >80%, >75%, >70%, >65%, >60%, >55% or >50% of X's of the structure of formula (II) are chemically-modified nucleotides.

### Structure of Formula (III)

In an embodiment, the compound of formula (I) has the structure of formula (III):

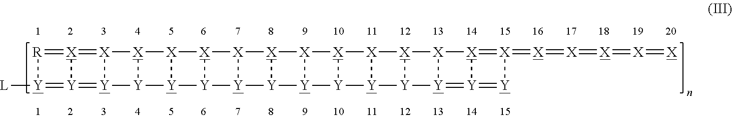

wherein X, for each occurrence, independently, is a nucleotide comprising a 2′-deoxy-2′-fluoro modification; X, for each occurrence, independently, is a nucleotide comprising a 2′-O-methyl modification; Y, for each occurrence, independently, is a nucleotide comprising a 2′-deoxy-2′-fluoro modification; and Y, for each occurrence, independently, is a nucleotide comprising a 2′-O-methyl modification.

In an embodiment, X is chosen from the group consisting of 2′-deoxy-2′-fluoro modified adenosine, guanosine, uridine or cytidine. In an embodiment, X is chosen from the group consisting of 2′-O-methyl modified adenosine, guanosine, uridine or cytidine. In an embodiment, Y is chosen from the group consisting of 2′-deoxy-2′-fluoro modified adenosine, guanosine, uridine or cytidine. In an embodiment, Y is chosen from the group consisting of 2′-O-methyl modified adenosine, guanosine, uridine or cytidine.

In certain embodiments, the structure of formula (III) does not contain mismatches. In one embodiment, the structure of formula (III) contains 1 mismatch. In another embodiment, the compound of formula (III) contains 2 mismatches. In another embodiment, the compound of formula (III) contains 3 mismatches. In another embodiment, the compound of formula (III) contains 4 mismatches.

### Structure of Formula (IV)

In an embodiment, the compound of formula (I) has the structure of formula (IV):

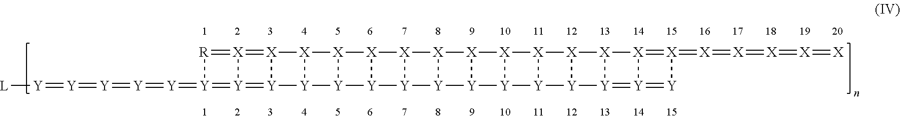

wherein X, for each occurrence, independently, is selected from adenosine, guanosine, uridine, cytidine, and chemically-modified derivatives thereof; Y, for each occurrence, independently, is selected from adenosine, guanosine, uridine, cytidine, and chemically-modified derivatives thereof; - represents a phosphodiester internucleoside linkage; ═ represents a phosphorothioate internucleoside linkage; and --- represents, individually for each occurrence, a base-pairing interaction or a mismatch.

In certain embodiments, the structure of formula (IV) does not contain mismatches. In one embodiment, the structure of formula (IV) contains 1 mismatch. In another embodiment, the compound of formula (IV) contains 2 mismatches. In another embodiment, the compound of formula (IV) contains 3 mismatches. In another embodiment, the compound of formula (IV) contains 4 mismatches. In an embodiment, each nucleic acid consists of chemically-modified nucleotides.

In certain embodiments, >95%, >90%, >85%, >80%, >75%, >70%, >65%, >60%, >55% or >50% of X's of the structure of formula (II) are chemically-modified nucleotides. In other embodiments, >95%, >90%, >85%, >80%, >75%, >70%, >65%, >60%, >55% or >50% of X's of the structure of formula (II) are chemically-modified nucleotides.

### Structure of Formula (V)

In an embodiment, the compound of formula (I) has the structure of formula (V):

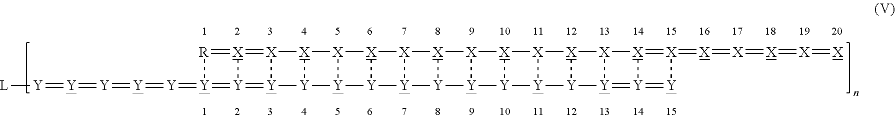

wherein X, for each occurrence, independently, is a nucleotide comprising a 2′-deoxy-2′-fluoro modification; X, for each occurrence, independently, is a nucleotide comprising a 2′-O-methyl modification; Y, for each occurrence, independently, is a nucleotide comprising a 2′-deoxy-2′-fluoro modification; and Y, for each occurrence, independently, is a nucleotide comprising a 2′-O-methyl modification.

In certain embodiments, X is chosen from the group consisting of 2′-deoxy-2′-fluoro modified adenosine, guanosine, uridine or cytidine. In an embodiment, X is chosen from the group consisting of 2′-O-methyl modified adenosine, guanosine, uridine or cytidine. In an embodiment, Y is chosen from the group consisting of 2′-deoxy-2′-fluoro modified adenosine, guanosine, uridine or cytidine. In an embodiment, Y is chosen from the group consisting of 2′-O-methyl modified adenosine, guanosine, uridine or cytidine.

In certain embodiments, the structure of formula (V) does not contain mismatches. In one embodiment, the structure of formula (V) contains 1 mismatch. In another embodiment, the compound of formula (V) contains 2 mismatches. In another embodiment, the compound of formula (V) contains 3 mismatches. In another embodiment, the compound of formula (V) contains 4 mismatches.

### Variable Linkers

In an embodiment of the compound of formula (I), L has the structure of L1:

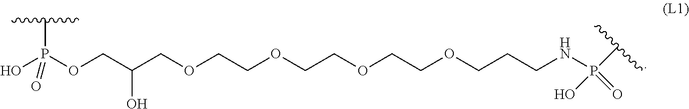 [SMILES: *P(=O)(O)NCCCOCCOCCOCCOCC(O)COP(*)(=O)O]

In an embodiment of L1, R is R³ and n is 2.

In an embodiment of the structure of formula (II), L has the structure of L1. In an embodiment of the structure of formula (III), L has the structure of L1. In an embodiment of the structure of formula (IV), L has the structure of L1. In an embodiment of the structure of formula (V), L has the structure of L1. In an embodiment of the structure of formula (VI), L has the structure of L1. In an embodiment of the structure of formula (VI), L has the structure of L1.

In an embodiment of the compound of formula (I), L has the structure of L2:

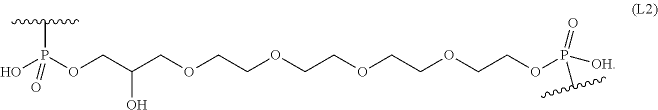 [SMILES: *P(=O)(O)OCCOCCOCCOCCOCC(O)COP(*)(=O)O]

In an embodiment of L2, R is R³ and n is 2. In an embodiment of the structure of formula (II), L has the structure of L2. In an embodiment of the structure of formula (III), L has the structure of L2. In an embodiment of the structure of formula (IV), L has the structure of L2. In an embodiment of the structure of formula (V), L has the structure of L2. In an embodiment of the structure of formula (VI), L has the structure of L2. In an embodiment of the structure of formula (VI), L has the structure of L2.

### 10) Delivery System

In a further aspect, provided herein is a delivery system for therapeutic nucleic acids having the structure of formula (VI):

L-(cNA)ₙ (VI)

wherein L is selected from an ethylene glycol chain, an alkyl chain, a peptide, RNA, DNA, a phosphate, a phosphonate, a phosphoramidate, an ester, an amide, a triazole, and combinations thereof, wherein formula (VI) optionally further comprises one or more branch point B, and one or more spacer S; wherein B is independently for each occurrence a polyvalent organic species or derivative thereof; S is independently for each occurrence selected from an ethylene glycol chain, an alkyl chain, a peptide, RNA, DNA, a phosphate, a phosphonate, a phosphoramidate, an ester, an amide, a triazole, and combinations thereof; each cNA, independently, is a carrier nucleic acid comprising one or more chemical modifications; and n is 2, 3, 4, 5, 6, 7 or 8.

In one embodiment of the delivery system, L is an ethylene glycol chain. In another embodiment of the delivery system, L is an alkyl chain. In another embodiment of the delivery system, L is a peptide. In another embodiment of the delivery system, L is RNA. In another embodiment of the delivery system, L is DNA. In another embodiment of the delivery system, L is a phosphate. In another embodiment of the delivery system, L is a phosphonate. In another embodiment of the delivery system, L is a phosphoramidate. In another embodiment of the delivery system, L is an ester. In another embodiment of the delivery system, L is an amide. In another embodiment of the delivery system, L is a triazole.

In one embodiment of the delivery system, S is an ethylene glycol chain. In another embodiment, S is an alkyl chain. In another embodiment of the delivery system, S is a peptide. In another embodiment, S is RNA. In another embodiment of the delivery system, S is DNA. In another embodiment of the delivery system, S is a phosphate. In another embodiment of the delivery system, S is a phosphonate. In another embodiment of the delivery system, S is a phosphoramidate. In another embodiment of the delivery system, S is an ester. In another embodiment, S is an amide. In another embodiment, S is a triazole.

In one embodiment of the delivery system, n is 2. In another embodiment of the delivery system, n is 3. In another embodiment of the delivery system, n is 4. In another embodiment of the delivery system, n is 5. In another embodiment of the delivery system, n is 6. In another embodiment of the delivery system, n is 7. In another embodiment of the delivery system, n is 8.

In certain embodiments, each cNA comprises >95%, >90%, >85%, >80%, >75%, >70%, >65%, >60%, >55% or >50% chemically-modified nucleotides.

In an embodiment, the compound of formula (VI) has a structure selected from formulas (VI-1)-(VI-9) of Table 3:

TABLE 3

| | |
|---|---|
| ANc—L—cNA | (VI-1) |
| ANc—S—L—S—cNA | (VI-2) |
| 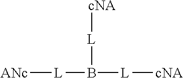 | (VI-3) |
| 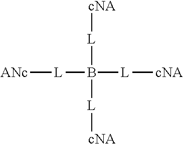 | (VI-4) |
| 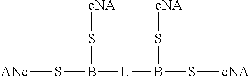 | (VI-5) |
| 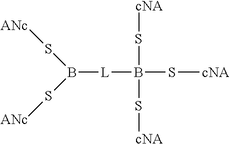 | (VI-6) |
| 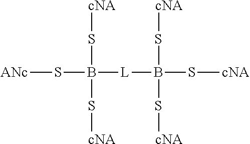 | (VI-7) |
| 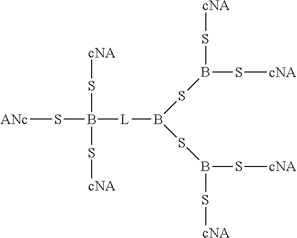 | (VI-8) |
| 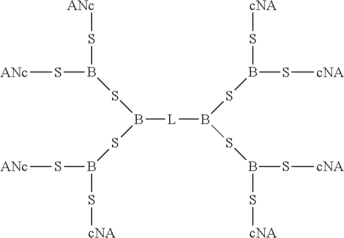 | (VI-9) |

In an embodiment, the compound of formula (VI) is the structure of formula (VI-1). In an embodiment, the compound of formula (VI) is the structure of formula (VI-2). In an embodiment, the compound of formula (VI) is the structure of formula (VI-3). In an embodiment, the compound of formula (VI) is the structure of formula (VI-4). In an embodiment, the compound of formula (VI) is the structure of formula (VI-5). In an embodiment, the compound of formula (VI) is the structure of formula (VI-6). In an embodiment, the compound of formula (VI) is the structure of formula (VI-7). In an embodiment, the compound of formula (VI) is the structure of formula (VI-8). In an embodiment, the compound of formula (VI) is the structure of formula (VI-9).

In an embodiment, the compound of formulas (VI) (including, e.g., formulas (VI-1)-(VI-9), each cNA independently comprises at least 15 contiguous nucleotides. In an embodiment, each cNA independently consists of chemically-modified nucleotides.

In an embodiment, the delivery system further comprises n therapeutic nucleic acids (NA), wherein each NA comprises a region of complementarity which is substantially complementary to a region of a gene comprising an allelic polymorphism, wherein the antisense strand comprises: a single nucleotide polymorphism (SNP) position nucleotide at a position 2 to 7 from the 5′ end that is complementary to the allelic polymorphism; and a mismatch (MM) position nucleotide located 2-11 nucleotide from the SNP position nucleotide that is a mismatch with a nucleotide in the gene. In exemplary embodiments, the SNP position nucleotide is at a position 2, 4 or 6 from the 5′ end and the mismatch (MM) position nucleotide is located 2-6 nucleotides from the SNP position nucleotide. Also, each NA is hybridized to at least one cNA. In one embodiment, the delivery system is comprised of 2 NAs. In another embodiment, the delivery system is comprised of 3 NAs. In another embodiment, the delivery system is comprised of 4 NAs. In another embodiment, the delivery system is comprised of 5 NAs. In another embodiment, the delivery system is comprised of 6 NAs. In another embodiment, the delivery system is comprised of 7 NAs. In another embodiment, the delivery system is comprised of 8 NAs.

In an embodiment, each NA independently comprises at least 16 contiguous nucleotides. In an embodiment, each NA independently comprises 16-20 contiguous nucleotides. In an embodiment, each NA independently comprises 16 contiguous nucleotides. In another embodiment, each NA independently comprises 17 contiguous nucleotides. In another embodiment, each NA independently comprises 18 contiguous nucleotides. In another embodiment, each NA independently comprises 19 contiguous nucleotides. In another embodiment, each NA independently comprises 20 contiguous nucleotides.

In an embodiment, each NA comprises an unpaired overhang of at least 2 nucleotides. In another embodiment, each NA comprises an unpaired overhang of at least 3 nucleotides. In another embodiment, each NA comprises an unpaired overhang of at least 4 nucleotides. In another embodiment, each NA comprises an unpaired overhang of at least 5 nucleotides. In another embodiment, each NA comprises an unpaired overhang of at least 6 nucleotides. In an embodiment, the nucleotides of the overhang are connected via phosphorothioate linkages.

In an embodiment, each NA, independently, is selected from the group consisting of: DNA, siRNAs, antagomiRs, miRNAs, gapmers, mixmers, or guide RNAs. In one embodiment, each NA, independently, is a DNA. In another embodiment, each NA, independently, is a siRNA. In another embodiment, each NA, independently, is an antagomiR. In another embodiment, each NA, independently, is a miRNA. In another embodiment, each NA, independently, is a gapmer. In another embodiment, each NA, independently, is a mixmer. In another embodiment, each NA, independently, is a guide RNA. In an embodiment, each NA is the same. In an embodiment, each NA is not the same.

In an embodiment, the delivery system further comprising n therapeutic nucleic acids (NA) has a structure selected from formulas (I), (II), (III), (IV), (V), (VI), and embodiments thereof described herein. In one embodiment, the delivery system has a structure selected from formulas (I), (II), (III), (IV), (V), (VI), and embodiments thereof described herein further comprising 2 therapeutic nucleic acids (NA). In another embodiment, the delivery system has a structure selected from formulas (I), (II), (III), (IV), (V), (VI), and embodiments thereof described herein further comprising 3 therapeutic nucleic acids (NA). In one embodiment, the delivery system has a structure selected from formulas (I), (II), (III), (IV), (V), (VI), and embodiments thereof described herein further comprising 4 therapeutic nucleic acids (NA). In one embodiment, the delivery system has a structure selected from formulas (I), (II), (III), (IV), (V), (VI), and embodiments thereof described herein further comprising 5 therapeutic nucleic acids (NA). In one embodiment, the delivery system has a structure selected from formulas (I), (II), (III), (IV), (V), (VI), and embodiments thereof described herein further comprising 6 therapeutic nucleic acids (NA). In one embodiment, the delivery system has a structure selected from formulas (I), (II), (III), (IV), (V), (VI), and embodiments thereof described herein further comprising 7 therapeutic nucleic acids (NA). In one embodiment, the delivery system has a structure selected from formulas (I), (II), (III), (IV), (V), (VI), and embodiments thereof described herein further comprising 8 therapeutic nucleic acids (NA).

In one embodiment, the delivery system has a structure selected from formulas (I), (II), (III), (IV), (V), (VI), further comprising a linker of structure L1 or L2 wherein R is R3 and n is 2. In another embodiment, the delivery system has a structure selected from formulas (I), (II), (III), (IV), (V), (VI), further comprising a linker of structure L1 wherein R is R3 and n is 2. In another embodiment, the delivery system has a structure selected from formulas (I), (II), (III), (IV), (V), (VI), further comprising a linker of structure L2 wherein R is R3 and n is 2.

### Pharmaceutical Compositions and Methods of Administration

In one aspect, provided herein is a pharmaceutical composition comprising a therapeutically effective amount of one or more compound, oligonucleotide, or nucleic acid as described herein, and a pharmaceutically acceptable carrier. In one embodiment, the pharmaceutical composition comprises one or more double-stranded, chemically-modified nucleic acid as described herein, and a pharmaceutically acceptable carrier. In a particular embodiment, the pharmaceutical composition comprises one double-stranded, chemically-modified nucleic acid as described herein, and a pharmaceutically acceptable carrier. In another particular embodiment, the pharmaceutical composition comprises two double-stranded, chemically-modified nucleic acids as described herein, and a pharmaceutically acceptable carrier.

In a particular embodiment, the pharmaceutical composition comprises a double-stranded RNA molecule comprising about 15-35 nucleotides complementary to a region of a gene encoding a heterozygous SNP mutant protein, said region comprising an allelic polymorphism, and a second strand comprising about 15-35 nucleotides complementary to the first strand, wherein the dsRNA molecule comprises a mismatch that is not in the position of the allelic polymorphism; and the mismatch and the nucleotide corresponding to the polymorphism are not in the center of the dsRNA molecule.

In an embodiment, the mismatch is 4 nucleotides upstream, 3 nucleotides upstream nucleotide corresponding to the allelic polymorphism, 2 nucleotides upstream nucleotide corresponding to the allelic polymorphism, 1 nucleotide upstream, 1 nucleotide downstream nucleotide corresponding to the allelic polymorphism, 2 nucleotides downstream nucleotide corresponding to the allelic polymorphism, 3 nucleotides downstream nucleotide corresponding to the allelic polymorphism, 4 nucleotides downstream nucleotide corresponding to the allelic polymorphism, or 5 nucleotides downstream nucleotide corresponding to the allelic polymorphism. In certain embodiments, the mismatch is not adjacent to the nucleotide corresponding to the allelic polymorphism.

In another embodiment of the pharmaceutical composition, the double-stranded RNA comprises a nucleotide corresponding to the allelic polymorphism which is in position 2, 3, 4, 5, or 6 from the 5′ end. In an embodiment, the nucleotide corresponding to the allelic polymorphism is in position 2 from the 5′ end. In an embodiment, the nucleotide corresponding to the allelic polymorphism is in position 3 from the 5′ end. In an embodiment, the nucleotide corresponding to the allelic polymorphism is in position 4 from the 5′ end. In an embodiment, the nucleotide corresponding to the allelic polymorphism is in position 5 from the 5′ end. In an embodiment, the nucleotide corresponding to the allelic polymorphism is in position 6 from the 5′ end.

In an embodiment of the pharmaceutical composition, the double-stranded RNA selectively silences a mutant allele having an allelic polymorphism, e.g., a heterozygous SNP. In an embodiment of the pharmaceutical composition, the double-stranded RNA silences a mutant allele having an allelic polymorphism and does not affect the wild-type allele of the same gene. In another embodiment of the pharmaceutical composition, the double-stranded RNA provided herein silences a mutant allele having an allelic polymorphism and silences the wild-type allele of the same gene to a lesser extent than the mutant allele.

A pharmaceutical composition of the disclosure is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desired to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, typical methods of preparation are vacuum drying and freeze-drying which yields a powder of the active ingredient plus any additional desired ingredient from a previously sterile-filtered solution thereof.

The data obtained from the cell culture assays and animal studies can be used in formulating a range of dosage for use in humans. The dosage of such compounds should typically lie within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

### Methods of Treatment

The present disclosure provides for both prophylactic and therapeutic methods of treating a subject at risk of (or susceptible to) a disease or disorder caused, in whole or in part, by an allelic polymorphism (e.g., a heterozygous SNP). In one embodiment, the disease or disorder is a trinucleotide repeat disease or disorder. In another embodiment, the disease or disorder is a polyglutamine disorder. In an embodiment, the methods comprise administering a therapeutically effective amount of a double-stranded RNA molecule provided herein. In an embodiment, the disease or disorder is a disorder associated with the expression of huntingtin and in which alteration of huntingtin, especially the amplification of CAG repeat copy number, leads to a defect in the huntingtin gene (structure or function) or huntingtin protein (structure or function or expression), such that clinical manifestations include those seen in Huntington's disease patients.

In embodiments of the methods, the double-stranded RNAs disclosed herein are homologous to an allelic polymorphism except for one mismatched oligonucleotide at a particular position relative to the nucleotide corresponding to the allelic polymorphism. In certain embodiments, the mismatch is within about 6 nucleotides of the nucleotide corresponding to the allelic polymorphism, within about 5 nucleotides of the nucleotide corresponding to the allelic polymorphism, within about 4 nucleotides of the nucleotide corresponding to the allelic polymorphism within about 3 nucleotide of the nucleotide corresponding to the allelic polymorphism, within about 2 nucleotide of the nucleotide corresponding to the allelic polymorphism, or within about 1 nucleotides of the nucleotide corresponding to the allelic polymorphism. In particularly exemplary embodiments, the mismatch is not adjacent to the nucleotide corresponding to the allelic polymorphism.

In another embodiment of the methods, the double-stranded RNA comprises a nucleotide corresponding to the allelic polymorphism which is in position 2, 3, 4, 5, or 6 from the 5′ end. In an embodiment, the nucleotide corresponding to the allelic polymorphism is in position 2 from the 5′ end. In an embodiment, the nucleotide corresponding to the allelic polymorphism is in position 3 from the 5′ end. In an embodiment, the nucleotide corresponding to the allelic polymorphism is in position 4 from the 5′ end. In an embodiment, the nucleotide corresponding to the allelic polymorphism is in position 5 from the 5′ end. In an embodiment, the nucleotide corresponding to the allelic polymorphism is in position 6 from the 5′ end.

In an embodiment of the methods, the dsRNA comprises a nucleotide corresponding to a polymorphism at position 6 from the 5′end and a mismatch at position 11 from the 5′ end. In an embodiment of the methods, the dsRNA comprises a nucleotide corresponding to a polymorphism at position 4 from the 5′ end and a mismatch at position 7 from the 5′ end.

In another embodiment of the methods, the double-stranded RNA selectively silences a mutant allele having an allelic polymorphism. In an embodiment, the double-stranded RNA silences a mutant allele having an allelic polymorphism and does not affect the wild-type allele of the same gene. In another embodiment, the double-stranded RNA silences a mutant allele having an allelic polymorphism and silences the wild-type allele of the same gene to a lesser extent than the mutant allele.

In an embodiment of the methods, the dsRNA comprises one or more VP intersubunit linkage modifications wherein the intersubunit linkage has the following formula:

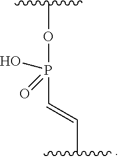 [SMILES: */C=C/P(=O)(O)O*]

In additional embodiments, the dsRNA comprises one or more of the intersubunit linkage modifications depicted in FIG. 43.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the disclosure pertains to methods treating subjects therapeutically, i.e., alter onset of symptoms of the disease or disorder. In an exemplary embodiment, the modulatory method of the disclosure involves contacting a cell expressing a gain-of-function mutant with a therapeutic agent (e.g., a RNAi agent or vector or transgene encoding same) that is specific for one or more target sequences within the gene, such that sequence specific interference with the gene is achieved. These methods can be performed in vitro (e.g., by culturing the cell with the agent) or, alternatively, in vivo (e.g., by administering the agent to a subject).

An RNA silencing agent modified for enhanced uptake into neural cells can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075 or 0.00015 nmole of RNA silencing agent per kg of bodyweight. The unit dose, for example, can be administered by injection (e.g., intravenous or intramuscular, intrathecally, or directly into the brain), an inhaled dose, or a topical application. In exemplary embodiments, dosages are less than 2, 1 or 0.1 mg/kg of body weight.

Delivery of an RNA silencing agent directly to an organ (e.g., directly to the brain) can be at a dosage on the order of about 0.00001 mg to about 3 mg per organ, or about 0.0001-0.001 mg per organ, about 0.03-3.0 mg per organ, about 0.1-3.0 mg per organ or about 0.3-3.0 mg per organ. The dosage can be an amount effective to treat or prevent a neurological disease or disorder (e.g., Huntington's disease). In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In one embodiment, a subject is administered an initial dose, and one or more maintenance doses of an RNA silencing agent. The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are typically administered no more than once every 5, 10, or 30 days. Further, the treatment regimen may last for a period of time which will vary depending upon the nature of the particular disease, its severity and the overall condition of the patient. In particular embodiments, the dosage may be delivered no more than once per day, e.g., no more than once per 24, 36, 48 or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

### Huntington's Disease

In certain aspects of the disclosure, RNA silencing agents are designed to target polymorphisms (e.g., heterozygous single nucleotide polymorphisms) in the mutant human huntingtin protein (htt) for the treatment of Huntington's disease. Accordingly, in another aspect, provided herein is a method of treating or managing Huntington's disease comprising administering to a patient in need of such treatment or management a therapeutically effective amount of a compound, oligonucleotide, or nucleic acid as described herein, or a pharmaceutical composition comprising said compound, oligonucleotide, or nucleic acid.

Huntington's disease, inherited as an autosomal dominant disease, causes impaired cognition and motor disease. Patients can live more than a decade with severe debilitation, before premature death from starvation or infection. The disease begins in the fourth or fifth decade for most cases, but a subset of patients manifest disease in teenage years. The genetic mutation for Huntington's disease is a lengthened CAG repeat in the huntingtin gene. The CAG repeat varies in number from 8 to 35 copies in normal individuals (Kremer et al., 1994). The genetic mutation (e.g., an increase in length of the CAG repeats from less than 36 in the normal huntingtin gene to greater than 36 in the disease) is associated with the synthesis of a mutant huntingtin protein, which has greater than 36 consecutive polyglutamine residues (Aronin et al., 1995). In general, individuals with 36 or more CAG repeats will get Huntington's disease. Prototypic for as many as twenty other diseases with a lengthened CAG as the underlying mutation, Huntington's disease still has no effective therapy. A variety of interventions-such as interruption of apoptotic pathways, addition of reagents to boost mitochondrial efficiency, and blockade of NMDA receptors—have shown promise in cell cultures and mouse model of Huntington's disease. However, at best these approaches reveal a short prolongation of cell or animal survival.

The disease gene linked to Huntington's disease is termed Huntingtin or (htt). The huntingtin locus is large, spanning 180 kb and consisting of 67 exons. The huntingtin gene is widely expressed and is required for normal development. It is expressed as 2 alternatively polyadenylated forms displaying different relative abundance in various fetal and adult tissues. The larger transcript is approximately 13.7 kb and is expressed predominantly in adult and fetal brain whereas the smaller transcript of approximately 10.3 kb is more widely expressed. The two transcripts differ with respect to their 3′ untranslated regions (Lin et al., 1993). Both messages are predicted to encode a 348 kilodalton protein containing 3144 amino acids. The genetic defect leading to Huntington's disease is believed to confer a new property on the mRNA or alter the function of the protein.

Huntington's disease complies with the central dogma of genetics: a mutant gene serves as a template for production of a mutant mRNA; the mutant mRNA then directs synthesis of a mutant protein (Aronin et al., 1995; DiFiglia et al., 1997). Mutant huntingtin (protein) likely accumulates in selective neurons in the striatum and cortex, disrupts as yet determined cellular activities, and causes neuronal dysfunction and death (Aronin et al., 1999; Laforet et al., 2001). Because a single copy of a mutant gene suffices to cause Huntington's disease, the most parsimonious treatment would render the mutant gene ineffective. Theoretical approaches might include stopping gene transcription of mutant huntingtin, destroying mutant mRNA, and blocking translation. Each has the same outcome-loss of mutant huntingtin.

### Huntington SNPs

Exemplary SNPs in the huntingtin gene sequence suitable for targeting according to certain exemplary embodiments are disclosed in Table 4 below. Genomic sequence for each SNP site can be found in, for example, the publicly available “SNP Entrez” database maintained by the NCBI. The frequency of heterozygosity for each SNP site for HD patient and control DNA is further illustrated in Table 4. Targeting combinations of frequently heterozygous SNPs allows the treatment of a large percentage of the individuals in a HD population using a relatively small number of allele-specific RNA silencing agents.

TABLE 4
htt SNPs.

| | | | |
|---|---|---|---|
| rs363125 | ORF, exon 39 | 11.00% | GTTAAGAGATGGGGAC |
| | | | AGTA[A/C]TTCAACG |
| | | | CTAGAAGAACACA |
| | | | (SEQ ID NO: 1) |
| rs362273 | ORF, exon 57 | 35.20% | AGCCACGAGAAGCTGC |
| | | | TGCT[A/G]CAGATCA |
| | | | ACCCCGAGCGGGA |
| | | | (SEQ ID NO: 2) |
| rs362307 | 3' UTR, exon 67 | 48.60% | CCGGAGCCTTTGGAAG |
| | | | TCTG[C/T]GCCCTTG |
| | | | TGCCCTGCCTCCA |
| | | | (SEQ ID NO: 3) |
| rs362336 | ORF, exon 48 | 37.40% | CAGCCCGAGCTGCCTG |
| | | | CAGA[A/G]CCGGCGG |
| | | | CCTACTGGAGCAA |
| | | | (SEQ ID NO: 4) |
| rs362331 | ORF, exon 50 | 39.40% | CCCACGCCTGCTCCCT |
| | | | CATC[C/T]ACTGTGT |
| | | | GCACTTCATCCTG |
| | | | (SEQ ID NO: 5) |
| rs362272 | ORF, exon 61 | 36.10% | GGGTTGGAGCCCTGCA |
| | | | CGGC[A/G]TCCTCTA |
| | | | TGTGCTGGAGTGC |
| | | | (SEQ ID NO: 6) |
| rs362306 | 3′ UTR, exon 67 | 35.80% | CTGCTGGTTGTTGCCA |
| | | | GGTT[A/G]CAGCTGC |
| | | | TCTTGCATCTGGG |
| | | | (SEQ ID NO: 7) |
| rs362268 | 3′ UTR, exon 67 | 35.80% | TCCTCCCTCCTGCAGG |
| | | | CTGG[C/G]TGTTGGC |
| | | | CCCTSTGCTGTCC |
| | | | (SEQ ID NO: 8) |
| rs362267 | 3′ UTR, exon 67 | 35.50% | GATTTGGGAGCTCTGC |
| | | | TTGC[C/TIGACTGGC |
| | | | TGTGAGACGAGGC |
| | | | (SEQ ID NO: 9) |
| rs363099 | ORF, exon 29 | 35.80% | GAAAAGTTTGGAGGGT |
| | | | TTCT[C/T]CGCTCAG |
| | | | CCTTGGATGTTCT |
| | | | (SEQ ID NO: 10) |

In one embodiment, RNA silencing agents of the disclosure are capable of targeting one or more of the SNP sites listed in Table 4. In one embodiment, RNA silencing agents of the disclosure are capable of targeting rs363125 SNP site of the Huntingtin mRNA. In another embodiment, RNA silencing agents of the disclosure are capable of targeting rs362273 SNP site of the Huntingtin mRNA. In another embodiment, RNA silencing agents of the disclosure are capable of targeting rs362307 SNP site of the Huntingtin mRNA. In another embodiment, RNA silencing agents of the disclosure are capable of targeting rs362336 SNP site of the Huntingtin mRNA. In another embodiment, RNA silencing agents of the disclosure are capable of targeting rs362331 SNP site of the Huntingtin mRNA. In another embodiment, RNA silencing agents of the disclosure are capable of targeting rs362272 SNP site of the Huntingtin mRNA. In another embodiment, RNA silencing agents of the disclosure are capable of targeting rs362306 SNP site of the Huntingtin mRNA. In another embodiment, RNA silencing agents of the disclosure are capable of targeting rs362268 SNP site of the Huntingtin mRNA. In another embodiment, RNA silencing agents of the disclosure are capable of targeting rs362267 SNP site of the Huntingtin mRNA. In another embodiment, RNA silencing agents of the disclosure are capable of targeting rs363099 SNP site of the Huntingtin mRNA. In some embodiments, SNP sites targeted by RNA silencing agents are associated with Huntington's Disease. In particularly exemplary embodiments, SNP sites targeted by RNA silencing agents are significantly associated with Huntington's Disease.

In additional exemplary embodiments, the RNA silencing agents include one or more of the sequences of Tables 5-7:

TABLE 5

| | | | addi- | | | | |
|---|---|---|---|---|---|---|---|
| | | | tional | | | | |
| | | | mis- | sequence | | | |
| | com- | sup | match | | SEQ | | SEQ |
| | pound | posi- | posi- | antisense | ID | sense | ID |
| HTT SNP | name | tion | tion | strand | NO: | strand | NO: |
| rs362273 | SNP2- | 2 | 7 | UUAGCAUCAG | 230 | AGAAGCUG | 242 |
| | 7 | | | CUUCUCGUGG | | CUGCUAA | |
| rs362273 | SNP4- | 4 | 7 | UUGUAGUAGC | 231 | AAGCUGCU | 243 |
| | 7 | | | AGCUUCUCGU | | GCUACAA | |
| rs362273 | SNP4- | 4 | 8 | UUGUAGCUGC | 232 | AAGCUGCU | 243 |
| | 8 | | | AGCUUCUCGU | | GCUACAA | |
| rs362273 | SNP4- | 4 | 15 | UUGUAGCAGC | 233 | AAGCUGCU | 243 |
| | 15 | | | AGCUACUCGU | | GCUACAA | |
| rs362273 | SNP6- | 6 | 5 | UUCUAUAGCA | 234 | GCUGCUGC | 244 |
| | 5A | | | GCAGCUUCUC | | UACAGAA | |
| rs362273 | SNP6- | 6 | 8 | UUCUGUAUCA | 235 | GCUGCUGC | 244 |
| | 8 | | | GCAGCUUCUC | | UACAGAA | |
| rs362273 | SNP6- | 6 | 11 | UUCUGUAGCA | 236 | GCUGCUGC | 244 |
| | 11 | | | UCAGCUUCUC | | UACAGAA | |
| rs362273 | SNP6- | 6 | 14 | UUCUGUAGCA | 237 | GCUGCUGC | 244 |
| | 14 | | | GCAUCUUCUC | | UACAGAA | |
| rs362273 | SNP6- | 6 | 16 | UUCUGUAGCA | 238 | GCUGCUGC | 244 |
| | 16 | | | GCAGCAUCUC | | UACAGAA | |
| rs362307 | SNP3- | 3 | 5 | UCGCGGACUU | 239 | UUUGGAAG | 245 |
| | 5G | | | CCAAAGGCUC | | UCCGCGA | |
| rs362307 | SNP3- | 3 | 7 | UCGCAGGCUU | 240 | UUUGGAAG | 246 |
| | 7G | | | CCAAAGGCUC | | CCUGCGA | |
| rs362307 | SNP3- | 3 | 8 | UCGCAGAUUU | 241 | UUUGGAAA | 247 |
| | 8 | | | CCAAAGGCUC | | UCUGCGA | |

U can be replaced with T

TABLE 6

| | | description | | | | | |
|---|---|---|---|---|---|---|---|
| | | of siRNA | | | | | |
| | | sequence | | | | | |
| | | flanking | | | | | |
| | | snp site | | | | | |
| | | | addi- | | | | |
| SNP | | | tional | | | | |
| of | | | mis- | sequence | | | |
| inter- | com- | snp | match | anti- | SEQ | | SEQ |
| est | pound | posi- | posi- | sense | ID | sense | ID |
| SNP | name | tion | tion | strand | NO: | strand | NO: |
| rs362273 | SNP2- | 2 | 7 | UUAGCAU | 230 | AGAAGCUG | 242 |
| (A) | 7 | | | CAGCUUC | | CUGCUAA | |
| | | | | UCGUGG | | | |
| rs362273 | SNP4- | 4 | 7 | UUGUAGU | 231 | AAGCUGCU | 243 |
| (A) | 7 | | | AGCAGCU | | GCUACAA | |
| | | | | UCUCGU | | | |
| rs362273 | SNP4- | 4 | 8 | UUGUAGC | 232 | AAGCUGCU | 243 |
| (A) | 8 | | | UGCAGCU | | GCUACAA | |
| | | | | UCUCGU | | | |
| rs362273 | SNP4- | 4 | 15 | UUGUAGC | 233 | AAGCUGCU | 243 |
| (A) | 15 | | | AGCAGCU | | GCUACAA | |
| | | | | ACUCGU | | | |
| rs362273 | SNP6- | 6 | 5 | UUCUAUA | 234 | GCUGCUGC | 244 |
| (A) | 5A | | | GCAGCAG | | UACAGAA | |
| | | | | CUUCUC | | | |
| rs362273 | SNP6- | 6 | 8 | UUCUGUA | 235 | GCUGCUGC | 244 |
| (A) | 8 | | | UCAGCAG | | UACAGAA | |
| | | | | CUUCUC | | | |
| rs362273 | SNP6- | 6 | 11 | UUCUGUA | 236 | GCUGCUGC | 244 |
| (A) | 11 | | | GCAUCAG | | UACAGAA | |
| | | | | CUUCUC | | | |
| rs362273 | SNP6- | 6 | 14 | UUCUGUA | 237 | GCUGCUGC | 244 |
| (A) | 14 | | | GCAGCAU | | UACAGAA | |
| | | | | CUUCUC | | | |
| rs362273 | SNP6- | 6 | 16 | UUCUGUA | 238 | GCUGCUGC | 244 |
| (A) | 16 | | | GCAGCAG | | UACAGAA | |
| | | | | CAUCUC | | | |
| rs362307 | SNP3- | 3 | 5 | UCGCGGA | 239 | UUUGGAAG | 245 |
| (C′) | 5G | | | CUUCCAA | | UCCGCGA | |
| | | | | AGGCUC | | | |
| rs362307 | SNP3- | 3 | 7 | UCGCAGG | 240 | UUUGGAAG | 246 |
| (C′) | 7G | | | CUUCCAA | | CCUGCGA | |
| | | | | AGGCUC | | | |
| rs362307 | SNP3- | 3 | 8 | UCGCAGA | 241 | UUUGGAAA | 247 |
| (C′) | 8 | | | UUUCCAA | | UCUGCGA | |
| | | | | AGGCUC | | | |

TABLE 7

| | | description | | | | | |
|---|---|---|---|---|---|---|---|
| | | of siRNA | | | | | |
| | | sequence | | | | | |
| | | flanking | | | | | |
| | | snp | | | | | |
| | | | addi- | | | | |
| | | | tional | | | | |
| SNP of | | | mis- | sequence | | | |
| inter- | com- | snp | match | anti- | SEQ | | SEQ |
| est | pound | posi- | posi- | sense | ID | sense | ID |
| SNP | name | tion | tion | strand | NO: | strand | NO: |
| rs362273 | SNP2- | 2 | 7 | UCAGCAU | 248 | AGAAGCUG | 259 |
| (G) | 7 | | | CAGCUUC | | CUGCUGA | |
| | | | | UCGUGG | | | |
| rs362273 | SNP4- | 4 | 7 | UUGCAGU | 249 | AAGCUGCU | 260 |
| (G) | 7 | | | AGCAGCU | | GCUGCAA | |
| | | | | UCUCGU | | | |
| rs362273 | SNP4- | 4 | 8 | UUGCAGC | 250 | AAGCUGCU | 260 |
| (G) | 8 | | | UGCAGCU | | GCUGCAA | |
| | | | | UCUCGU | | | |
| rs362273 | SNP4- | 4 | 15 | UUGCAGC | 251 | AAGCUGCU | 260 |
| (G) | 15 | | | AGCAGCU | | GCUGCAA | |
| | | | | ACUCGU | | | |
| rs362273 | SNP6- | 6 | 5 | UUCUACA | 252 | GCUGCUGC | 261 |
| (G) | 5A | | | GCAGCAG | | UGCAGAA | |
| | | | | CUUCUC | | | |
| rs362273 | SNP6- | 6 | 8 | UUCUGCA | 253 | GCUGCUGC | 261 |
| (G) | 8 | | | UCAGCAG | | UGCAGAA | |
| | | | | CUUCUC | | | |
| rs362273 | SNP6- | 6 | 11 | UUCUGCA | 254 | GCUGCUGC | 261 |
| (G) | 11 | | | GCAUCAG | | UGCAGAA | |
| | | | | CUUCUC | | | |
| rs362273 | SNP6- | 6 | 14 | UUCUGCA | 255 | GCUGCUGC | 261 |
| (G) | 14 | | | GCAGCAU | | UGCAGAA | |
| | | | | CUUCUC | | | |
| rs362307 | SNP3- | 3 | 5 | UCACGGA | 256 | UUUGGAAG | 262 |
| (T) | 5G | | | CUUCCAA | | UCCGUGA | |
| | | | | AGGCUC | | | |
| rs362307 | SNP3- | 3 | 7 | UCACAGG | 257 | UUUGGAAG | 263 |
| (T) | 7G | | | CUUCCAA | | CCUGUGA | |
| | | | | AGGCUC | | | |
| rs362307 | SNP3- | 3 | 8 | UCACAGA | 258 | UUUGGAAA | 264 |
| (T) | 8 | | | UUUCCAA | | UCUGUGA | |
| | | | | AGGCUC | | | |

In certain embodiments of Tables 5-7, a U nucleotide may be replaced with a T nucleotide.

### Methods of Delivering Nucleic Acids

RNA silencing agents of the disclosure may be directly introduced into a cell (e.g., a neural cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-IS mediated transport, such as calcium phosphate, and the like. Thus, the RNA may be introduced along with components that perform one or more of the following activities: enhance RNA uptake by the cell, inhibit annealing of single strands, stabilize the single strands, or other-wise increase inhibition of the target gene.

RNA may be directly introduced into the cell (i.e., intracellularly), or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the RNA may be introduced.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double-stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS) and the like.

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Depending on the assay, quantitation of the amount of gene expression allows one to determine a degree of inhibition which is greater than 10%, 33%, 50%, 90%, 95% or 99% as compared to a cell not treated according to the present disclosure. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell. mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In a particular aspect, the efficacy of an RNAi agent of the disclosure (e.g., an siRNA targeting a polymorphism in a mutant gene) is tested for its ability to specifically degrade mutant mRNA (e.g., mutant htt mRNA and/or the production of mutant huntingtin protein) in cells, in particular, in neurons (e.g., striatal or cortical neuronal clonal lines and/or primary neurons). Also suitable for cell-based validation assays are other readily transfectable cells, for example, HeLa cells or COS cells. Cells are transfected with human wild-type or mutant cDNAs (e.g., human wild-type or mutant huntingtin cDNA). Standard siRNA, modified siRNA or vectors able to produce siRNA from U-looped mRNA are co-transfected. Selective reduction in mutant mRNA (e.g., mutant huntingtin mRNA) and/or mutant protein (e.g., mutant huntingtin) is measured. Reduction of mutant mRNA or protein can be compared to levels of normal mRNA or protein. Exogenously-introduced normal mRNA or protein (or endogenous normal mRNA or protein) can be assayed for comparison purposes. When utilizing neuronal cells, which are known to be somewhat resistant to standard transfection techniques, it may be desirable to introduce RNAi agents (e.g., siRNAs) by passive uptake.

In certain exemplary embodiments, a composition that includes an RNA agent, e.g., a dsRNA agent, of the disclosure can be delivered to the nervous system of a subject by a variety of routes. Exemplary routes include intrathecal, parenchymal (e.g., in the brain), nasal, and ocular delivery. The composition can also be delivered systemically, e.g., by intravenous, subcutaneous or intramuscular injection, which is particularly useful for delivery of the RNA agents, e.g., dsRNA agents, to peripheral neurons. An exemplary route of delivery is directly to the brain, e.g., into the ventricles or the hypothalamus of the brain, or into the lateral or dorsal areas of the brain. The RNA agents, e.g., dsRNA agents, for neural cell delivery can be incorporated into pharmaceutical compositions suitable for administration.

For example, compositions can include one or more species of an RNA agent, e.g., a dsRNA agent, and a pharmaceutically acceptable carrier. The pharmaceutical compositions of the present disclosure may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, intrathecal, or intraventricular (e.g., intracerebroventricular) administration. In certain exemplary embodiments, an RNA silencing agent of the disclosure is delivered across the Blood-Brain-Barrier (BBB) suing a variety of suitable compositions and methods described herein.

The route of delivery can be dependent on the disorder of the patient. For example, a subject diagnosed with Huntington's disease can be administered an anti-htt RNA agent, e.g., a dsRNA agent, of the disclosure directly into the brain (e.g., into the globus pallidus or the corpus striatum of the basal ganglia, and near the medium spiny neurons of the corpus striatum). In addition to an RNA silencing agent of the disclosure, a patient can be administered a second therapy, e.g., a palliative therapy and/or disease-specific therapy. The secondary therapy can be, for example, symptomatic (e.g., for alleviating symptoms), neuroprotective (e.g., for slowing or halting disease progression), or restorative (e.g., for reversing the disease process). For the treatment of Huntington's disease, for example, symptomatic therapies can include the drugs haloperidol, carbamazepine, or valproate. Other therapies can include psychotherapy, physiotherapy, speech therapy, communicative and memory aids, social support services, and dietary advice.

An RNA agent, e.g., a dsRNA agent, can be delivered to neural cells of the brain. Delivery methods that do not require passage of the composition across the blood-brain barrier can be utilized. For example, a pharmaceutical composition containing an RNA agent, e.g., a dsRNA agent, can be delivered to the patient by injection directly into the area containing the disease-affected cells. For example, the pharmaceutical composition can be delivered by injection directly into the brain. The injection can be by stereotactic injection into a particular region of the brain (e.g., the substantia nigra, cortex, hippocampus, striatum, or globus pallidus). The RNA agent, e.g., a dsRNA agent, can be delivered into multiple regions of the central nervous system (e.g., into multiple regions of the brain, and/or into the spinal cord). The RNA agent, e.g., a dsRNA agent, can be delivered into diffuse regions of the brain (e.g., diffuse delivery to the cortex of the brain).

In one embodiment, the RNA agent, e.g., a dsRNA agent, can be delivered by way of a cannula or other delivery device having one end implanted in a tissue, e.g., the brain, e.g., the substantia nigra, cortex, hippocampus, striatum or globus pallidus of the brain. The cannula can be connected to a reservoir of RNA agent, e.g., dsRNA agent. The flow or delivery can be mediated by a pump, e.g., an osmotic pump or minipump, such as an Alzet pump (Durect, Cupertino, CA). In one embodiment, a pump and reservoir are implanted in an area distant from the tissue, e.g., in the abdomen, and delivery is affected by a conduit leading from the pump or reservoir to the site of release. Devices for delivery to the brain are described, for example, in U.S. Pat. Nos. 6,093,180, and 5,814,014.

An RNA agent, e.g., a dsRNA agent, of the disclosure can be further modified such that it is capable of traversing the blood brain barrier. For example, the RNA agent, e.g., a dsRNA agent, can be conjugated to a molecule that enables the agent to traverse the barrier. Such modified RNA agents, e.g., dsRNA agents, can be administered by any desired method, such as by intraventricular or intramuscular injection, or by pulmonary delivery, for example.

In certain embodiments, exosomes are used to deliver an RNA agent, e.g., a dsRNA agent, of the disclosure. Exosomes can cross the BBB and deliver siRNAs, antisense oligonucleotides, chemotherapeutic agents and proteins specifically to neurons after systemic injection (See, Alvarez-Erviti L, Seow Y, Yin H, Betts C, Lakhal S, Wood M J. (2011). Delivery of siRNA to the mouse brain by systemic injection of targeted exosomes. Nat Biotechnol. 2011 April; 29(4): 341-5. doi: 10.1038/nbt.1807; El-Andaloussi S, Lee Y, Lakhal-Littleton S, Li J, Scow Y, Gardiner C, Alvarez-Erviti L, Sargent I L, Wood M J. (2011). Exosome-mediated delivery of siRNA in vitro and in vivo. Nat Protoc. 2012 December; 7 (12): 2112-26. doi: 10.1038/nprot.2012.131; EL Andaloussi S, Mager I, Breakefield X O, Wood M J. (2013). Extracellular vesicles: biology and emerging therapeutic opportunities. Nat Rev Drug Discov. 2013 May; 12(5): 347-57. doi: 10.1038/nrd3978; El Andaloussi S, Lakhal S, Mäger I, Wood M J. (2013). Exosomes for targeted siRNA delivery across biological barriers. Adv. Drug Deliv Rev. 2013 March; 65(3): 391-7. doi: 10.1016/j.addr.2012.08.008).

In certain embodiments, one or more lipophilic molecules are used to allow delivery of an RNA agent, e.g., a dsRNA agent, of the disclosure past the BBB (Alvarez-Ervit (2011)). The RNA silencing agent would then be activated, e.g., by enzyme degradation of the lipophilic disguise to release the drug into its active form.

In certain embodiments, one or more receptor-mediated permeabilizing compounds can be used to increase the permeability of the BBB to allow delivery of an RNA silencing agent of the disclosure. These drugs increase the permeability of the BBB temporarily by increasing the osmotic pressure in the blood which loosens the tight junctions between the endothelial cells ((El-Andaloussi (2012)). By loosening the tight junctions normal intravenous injection of an RNA silencing agent can be performed.

In certain embodiments, nanoparticle-based delivery systems are used to deliver an RNA agent, e.g., a dsRNA agent, of the disclosure across the BBB. As used herein, “nanoparticles” refer to polymeric nanoparticles that are typically solid, biodegradable, colloidal systems that have been widely investigated as drug or gene carriers (S. P. Egusquiaguirre, M. Igartua, R. M. Hernandez, and J. L. Pedraz, “Nanoparticle delivery systems for cancer therapy: advances in clinical and preclinical research,” Clinical and Translational Oncology, vol. 14, no. 2, pp. 83-93, 2012). Polymeric nanoparticles are classified into two major categories, natural polymers and synthetic polymers. Natural polymers for siRNA delivery include, but are not limited to, cyclodextrin, chitosan, and atelocollagen (Y. Wang, Z. Li, Y. Han, L. H. Liang, and A. Ji, “Nanoparticle-based delivery system for application of siRNA in vivo,” Current Drug Metabolism, vol. 11, no. 2, pp. 182-196, 2010). Synthetic polymers include, but are not limited to, polyethyleneimine (PEI), poly(dl-lactide-co-glycolide) (PLGA), and dendrimers, which have been intensively investigated (X. Yuan, S. Naguib, and Z. Wu, “Recent advances of siRNA delivery by nanoparticles,” Expert Opinion on Drug Delivery, vol. 8, no. 4, pp. 521-536, 2011). For a review of nanoparticles and other suitable delivery systems, See Jong-Min Lee, Tac-Jong Yoon, and Young-Seok Cho, “Recent Developments in Nanoparticle-Based siRNA Delivery for Cancer Therapy,” BioMed Research International, vol. 2013, Article ID 782041, 10 pages, 2013. doi: 10.1155/2013/782041 (incorporated by reference in its entirety.)

An RNA agent, e.g., a dsRNA agent, of the disclosure can be administered ocularly, such as to treat retinal disorder, e.g., a retinopathy. For example, the pharmaceutical compositions can be applied to the surface of the eye or nearby tissue, e.g., the inside of the eyelid. They can be applied topically, e.g., by spraying, in drops, as an eyewash, or an ointment. Ointments or droppable liquids may be delivered by ocular delivery systems known in the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The pharmaceutical composition can also be administered to the interior of the eye, and can be introduced by a needle or other delivery device which can introduce it to a selected area or structure. The composition containing the RNA silencing agent can also be applied via an ocular patch.

In general, an RNA agent, e.g., a dsRNA agent, of the disclosure can be administered by any suitable method. As used herein, topical delivery can refer to the direct application of an RNA agent, e.g., a dsRNA agent, to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the RNA agent, e.g., a dsRNA agent, to the epidermis or dermis of a subject, or to specific strata thereof, or to an underlying tissue.

Compositions for intrathecal or intraventricular (e.g., intracerebroventricular) administration may include sterile aqueous solutions which may also contain buffers, diluents and other suitable additives. Compositions for intrathecal or intraventricular administration typically do not include a transfection reagent or an additional lipophilic moiety besides, for example, the lipophilic moiety attached to the RNA agent, e.g., a dsRNA agent.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

An RNA agent, e.g., a dsRNA agent, of the disclosure can be administered to a subject by pulmonary delivery. Pulmonary delivery compositions can be delivered by inhalation of a dispersion so that the composition within the dispersion can reach the lung where it can be readily absorbed through the alveolar region directly into blood circulation. Pulmonary delivery can be effective both for systemic delivery and for localized delivery to treat diseases of the lungs. In one embodiment, an RNA agent, e.g., a dsRNA agent, administered by pulmonary delivery has been modified such that it is capable of traversing the blood brain barrier.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are exemplary. One of the benefits of using an atomizer or inhaler is that the potential for contamination is minimized because the devices are self-contained. Dry powder dispersion devices, for example, deliver drugs that may be readily formulated as dry powders. An RNA silencing agent composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The types of pharmaceutical excipients that are useful as carriers include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Bulking agents that are particularly valuable include compatible carbohydrates, polypeptides, amino acids or combinations thereof. Suitable carbohydrates include monosaccharides such as galactose, D-mannose, sorbose, and the like; disaccharides, such as lactose, trehalose, and the like; cyclodextrins, such as 2-hydroxypropyl-.beta.-cyclodextrin; and polysaccharides, such as raffinose, maltodextrins, dextrans, and the like; alditols, such as mannitol, xylitol, and the like. An exemplary group of carbohydrates includes lactose, trehalose, raffinose maltodextrins, and mannitol. Suitable polypeptides include aspartame. Amino acids include alanine and glycine, with glycine being exemplary.

Suitable pH adjusters or buffers include organic salts prepared from organic acids and bases, such as sodium citrate, sodium ascorbate, and the like; sodium citrate is exemplary.

An RNA agent, e.g., a dsRNA agent, of the disclosure can be administered by oral and nasal delivery. For example, drugs administered through these membranes have a rapid onset of action, provide therapeutic plasma levels, avoid first pass effect of hepatic metabolism, and avoid exposure of the drug to the hostile gastrointestinal (GI) environment. Additional advantages include easy access to the membrane sites so that the drug can be applied, localized and removed easily. In one embodiment, an RNA silencing agent administered by oral or nasal delivery has been modified to be capable of traversing the blood-brain barrier.

In one embodiment, unit doses or measured doses of a composition that include RNA agents, e.g., dsRNA agents, are dispensed by an implanted device. The device can include a sensor that monitors a parameter within a subject. For example, the device can include a pump, such as an osmotic pump and, optionally, associated electronics.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1: SNP Discrimination Varies According to the Position of the Mismatch

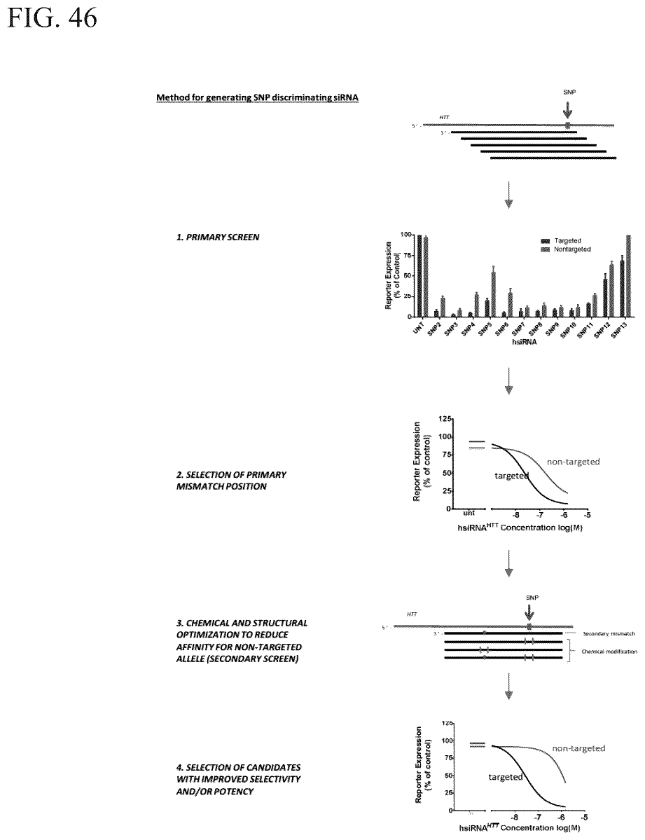

FIG. 46 is a flow chart illustrating a methodology for generating and selecting SNP-discriminating siRNAs that was implemented in the instance of HTT, but is also applicable to SNPs in other genes. A primary screen is conducted to determine which position the SNP is placed at causes the greatest discrimination. Then, the mismatch position(s) yielding best results are selected, and affinity for non-target alleles is further reduced in a secondary screening where chemical and structural optimizations to the siRNA molecule with improved selectivity and/or potency are selected.

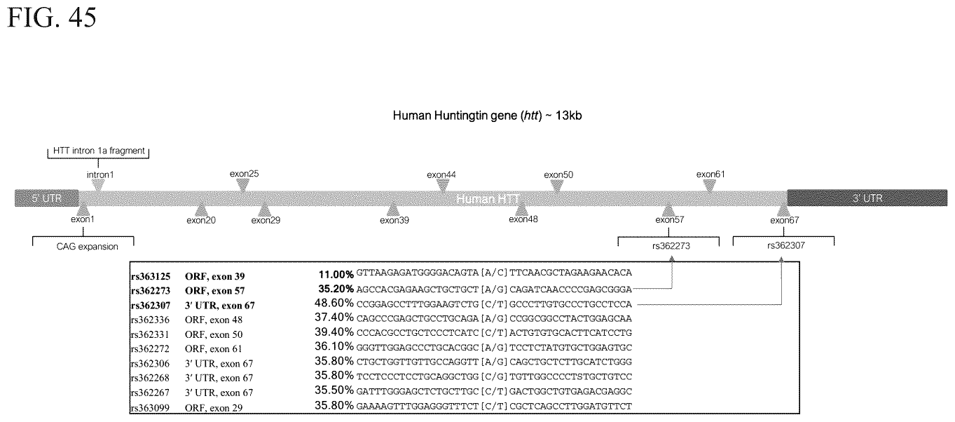

There are several SNPs within the HTT gene that have high rates of heterozygosity in HD patients (FIG. 45). For optimization of SNP-specific RNAi-mediated silencing of huntingtin, SNP rs362273 in exon 57 of HTT mRNA was used as model target for optimization of SNP selective silencing. This SNP heterozygosity occurs in 35% of the HD patient population.

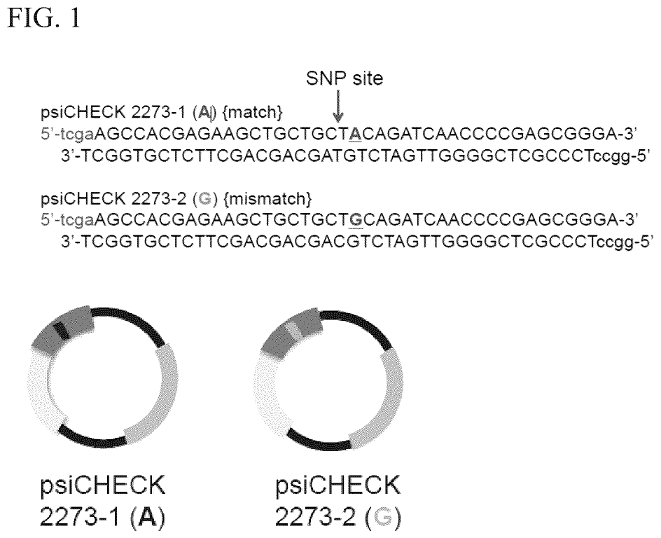

The psiCHECK reporter plasmid described herein contains SNP rs362273 and a partial flanking region from exon 57 of htt, within a Rluc 3′ UTR. The wild-type psiCHECK reporter plasmid contains the same region of htt without the SNP (FIG. 1).

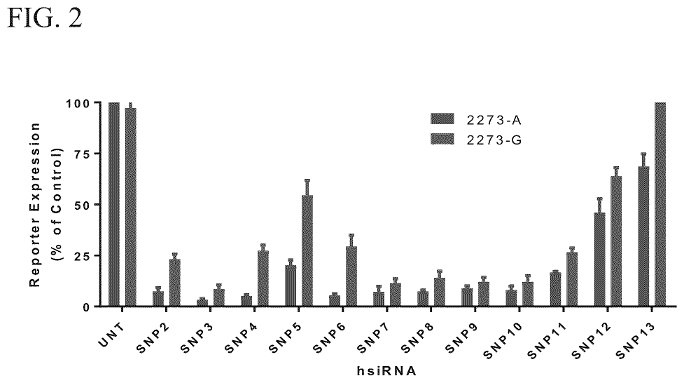

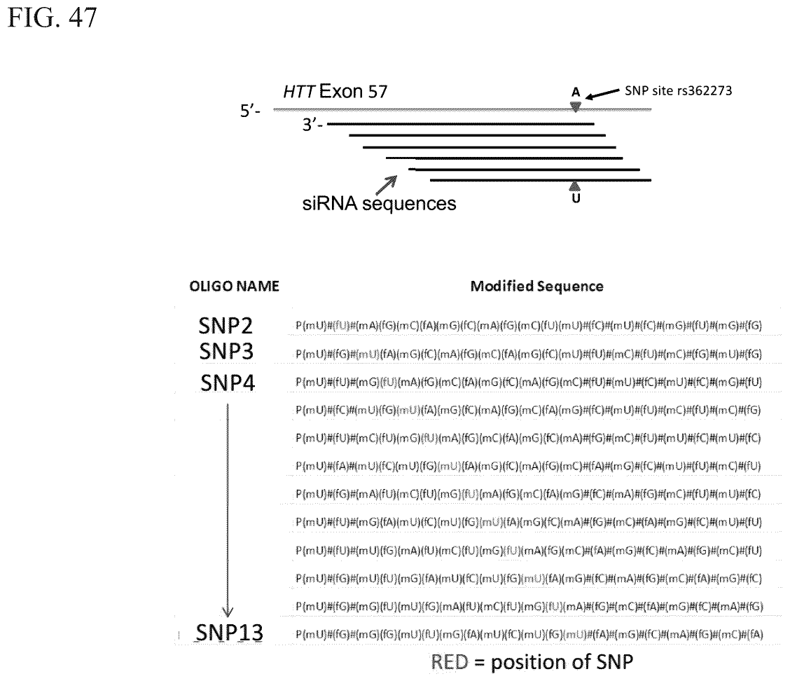

Hydrophobically modified RNAs (hsiRNAs) designed to be complimentary to the Huntingtin (htt) mRNA containing the mutant SNP (2273-1 (A)) were screened for efficacy with the psiCheck reporter plasmid system. The number following SNP represents the position of the SNP in the siRNA (FIG. 47). FIG. 2 shows that placing the SNP in position 2, 4 or 6 provided the greatest SNP discrimination, without losing efficacy against the mutant allele. Hela cells transfected with one of two reporter plasmids were reverse transfected with 1.5 μM hsiRNAs by passive uptake, and treated for 72 hours. Luciferase activity was measured at 72 hours post transfection (FIG. 2).

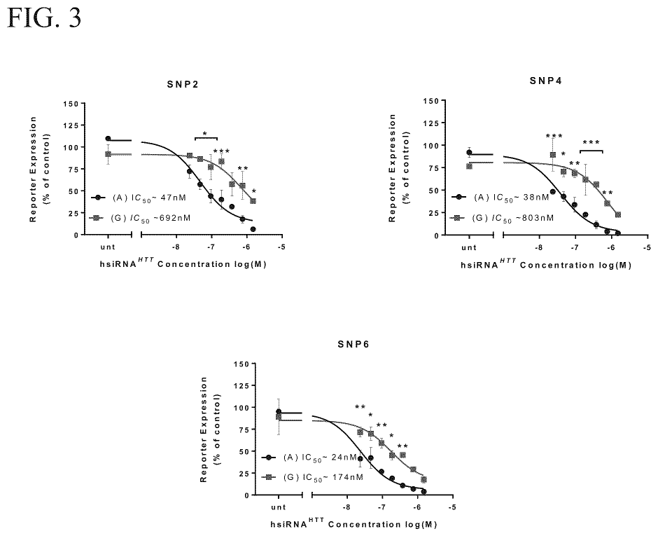

The hsiRNAs were further tested for allelic discrimination in a dose response dual luciferase assay in HeLa cells (FIG. 3). Multiple hsiRNAs preferentially silenced the reporter plasmid containing the mutant SNP as compared to the wild-type reporter plasmid. HeLa cells transfected with one of two reporter plasmids were reverse transfected with 1.5 μM hsiRNAs by passive uptake, and treated for 72 hours. Reporter plasmid expression was measured at 72 hours post transfection (FIG. 3).

### Example 2: SNP Discrimination in the Endogenous Htt mRNA

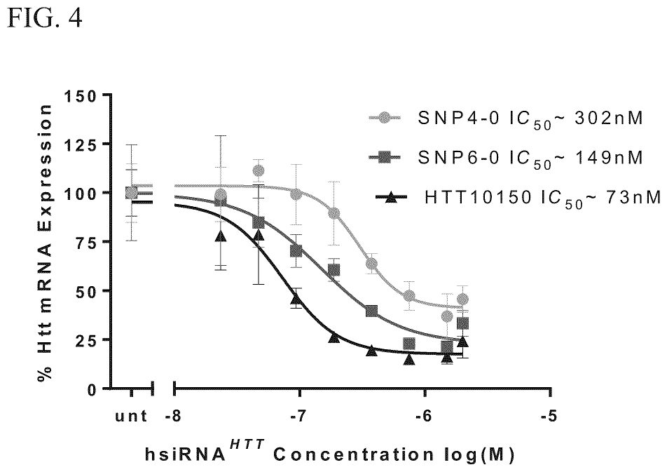

The hsiRNAs were tested for efficacy against the endogenous Huntingtin mRNA containing a homozygous rs362273 SNP. As HeLa cells are homozygous at rs362273, with an A on each allele, allelic discrimination was not assessed with this assay. Instead, FIG. 4 shows that two hsiRNAs, SNP4-0 and SNP6-0, were highly effective at silencing the htt mRNA containing the correct SNP. The mRNA levels were measured using Quantigene 2.0 bDNA assay after treating HeLa cells with hsiRNAs via passive uptake for 72 hours. Human htt mRNA levels were normalized to human HPRT.

### Example 3: Designing hsiRNAs with a Second Mismatch for Greater Allelic Discrimination

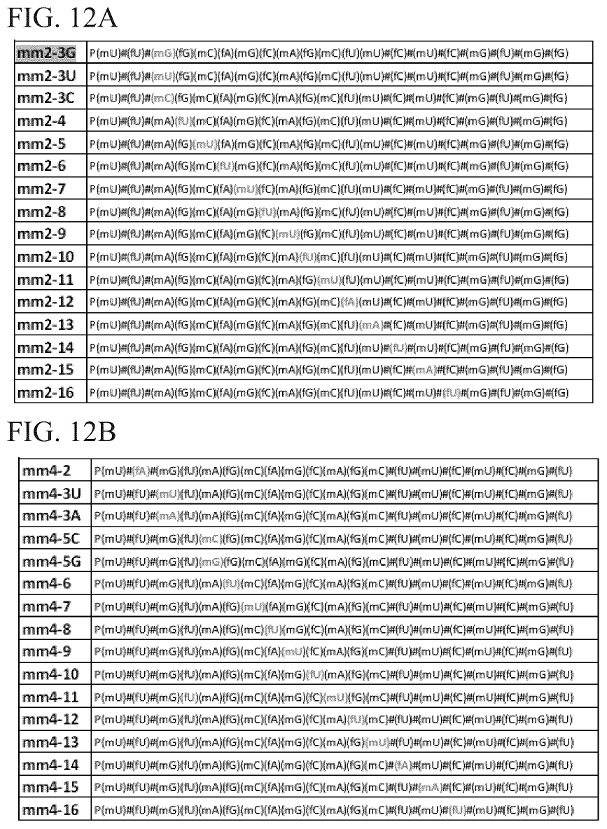

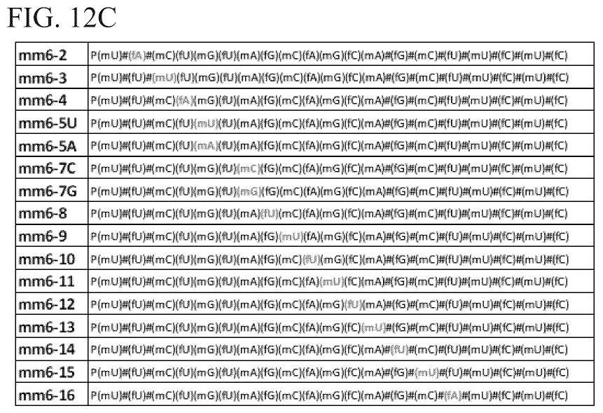

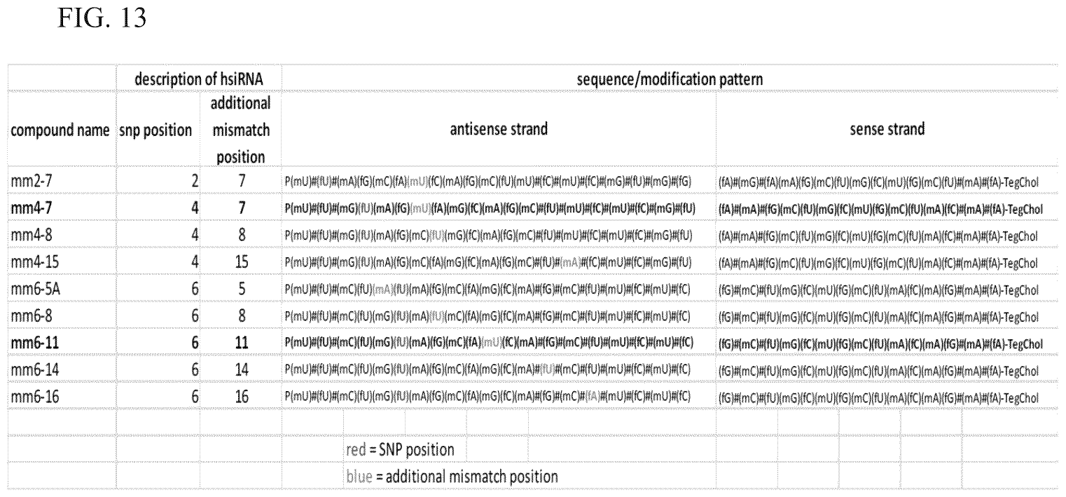

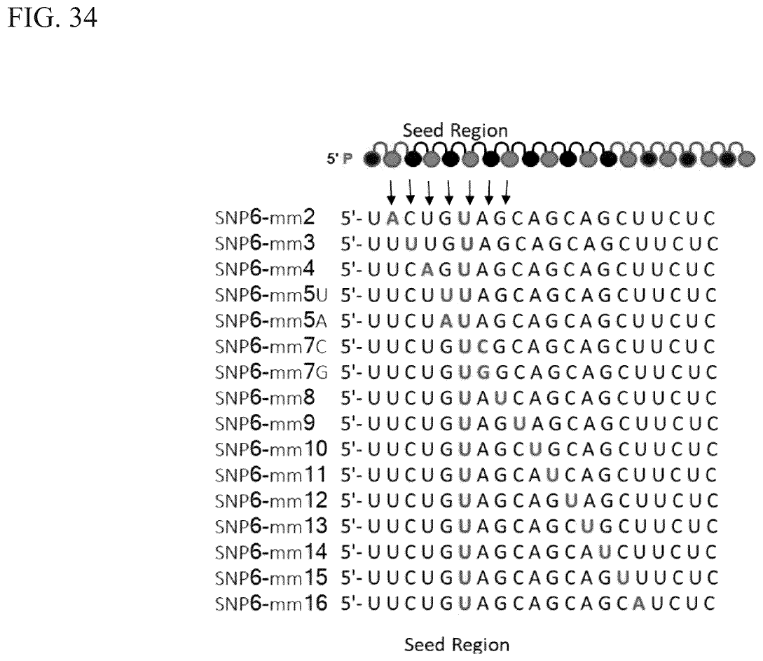

For each of the three hsiRNAs (SNP2-0, SNP4-0, and SNP6-0, also named mm2, mm4, and mm6, respectively) previously chosen for dose response, 16 new hsiRNAs were designed and synthesized with slight sequence modifications (FIG. 34). These sequences introduced a single mismatch at every possible position along the original sequence, in order to test if the second mismatch impairs silencing of the off-target SNP more significantly than before, with little effect on silencing the target SNP. Antisense strand sequences shown 5′ to 3′, with the SNP site in red, and the new mismatch in blue (FIG. 12).

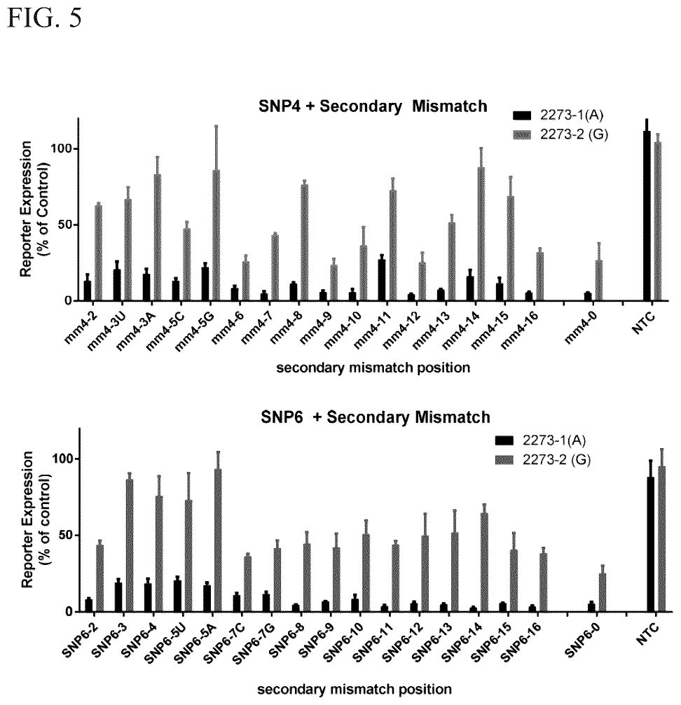

A primary screen of the efficacy of the hsiRNAs in FIG. 12 showed that the position of the second mismatch, relative to the position of the nucleotide corresponding to the SNP, resulted in varying levels of SNP discrimination in Hela cells. Hela cells transfected with one of two psiCHECK reporter plasmids were reverse transfected with 1.5 μM hsiRNAs by passive uptake, and treated for 72 hours. Luciferase activity was measured at 72 hours post transfection. FIG. 5 shows that multiple hsiRNAs discriminately silenced the reporter plasmid containing the SNP mutation as compared to the wild-type reporter plasmid.

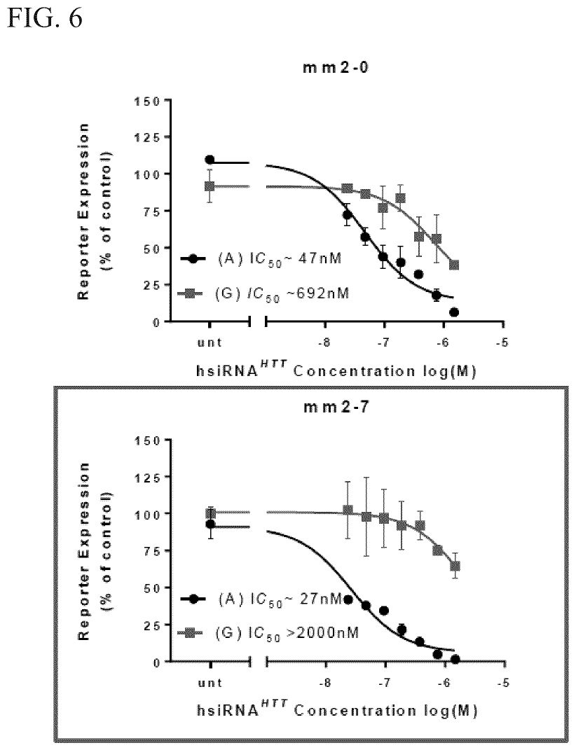

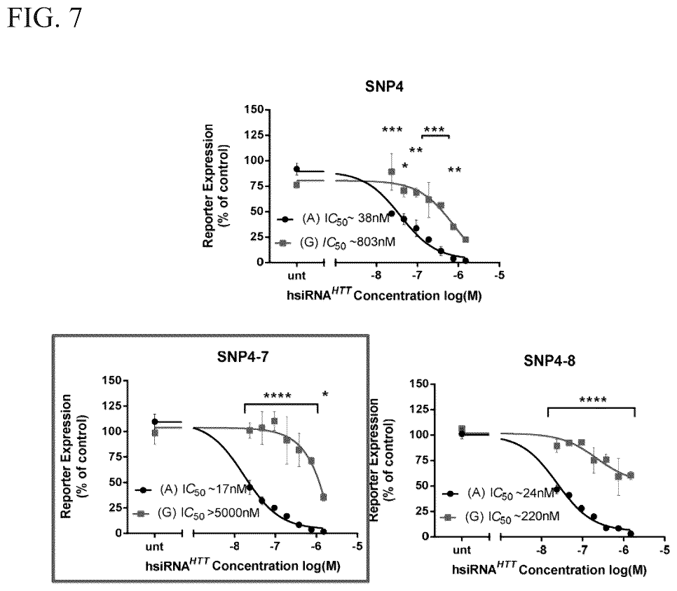

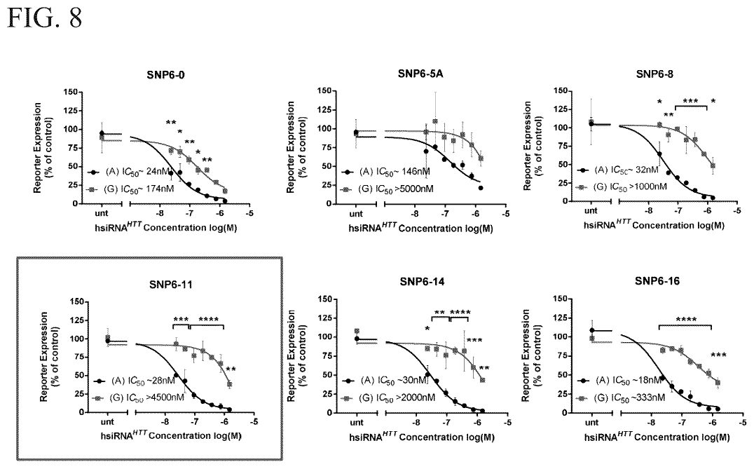

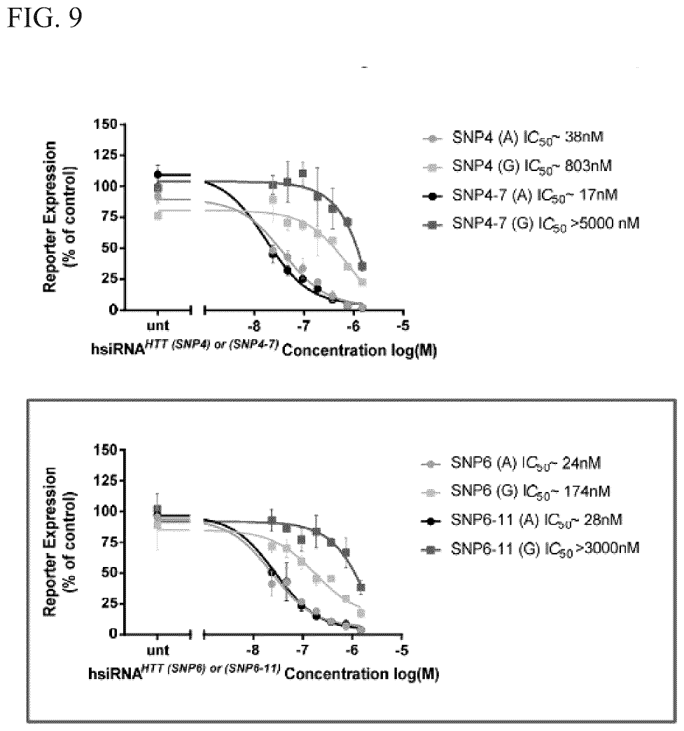

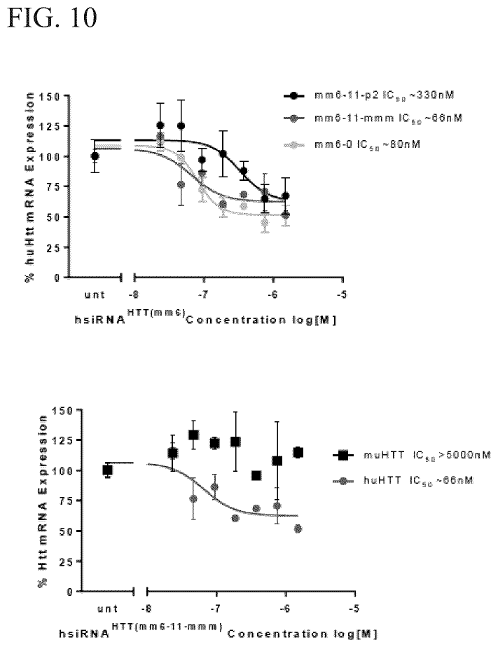

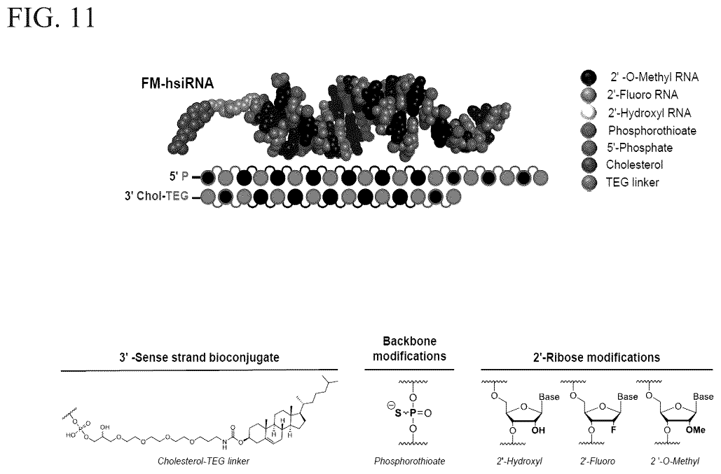

The most efficacious hsiRNAs, containing the second mismatch, were further tested in a dose response curve to verify improved SNP discrimination. HeLa cells transfected with one of two reporter plasmids were reverse transfected with hsiRNAs by passive uptake, and treated for 72 hours. Reporter expression measured with a dual-luciferase assay. FIGS. 6-8 show the IC50 values of the hsiRNAs with two mismatches for silencing the reporter plasmid containing the SNP mutation versus the wild-type reporter plasmid. The SNP6-11 hsiRNA (hsiRNA molecule with the nucleotide corresponding to the polymorphism at position 6 from the 5′ end and the mismatch at position 11 from the 5′ end) and the SNP4-7 hsiRNA (hsiRNA molecule with the nucleotide corresponding to the polymorphism at position 4 from the 5′end and the mismatch at position 7 from the 5′ end) were shown to be the most efficacious (see FIGS. 7-9). Surprisingly, altering the modification pattern around the SNP rescues efficacy lost by introducing the second mismatch without impairing discrimination. The SNP6-11 hsiRNA was altered so that it had 2′O-methyl modifications flanking the mismatch nucleotide (as well as the mismatch nucleotide itself having the 2′O-methyl modification) (see FIG. 10).

### Example 4: Additional Modifications

A variety of oligonucleotide types (e.g., gapmers, mixmers, miRNA inhibitors, splice-switching oligonucleotides (“SSOs”), phosphorodiamidate morpholino oligonucleotides (“PMOs”), peptide nucleic acids (“PNAs”) and the like) can be used in the oligonucleotides described herein, optionally utilizing various combinations of modifications (e.g., chemical modifications) and/or conjugations described herein and in, e.g., U.S. Ser. No. 15/089,423; U.S. Ser. No. 15/236,051; U.S. Ser. No. 15/419,593; U.S. Ser. No. 15/697,120 and U.S. Pat. No. 9,809,817; and U.S. Ser. No. 15/814,350 and U.S. Pat. No. 9,862,350, each of which is incorporated herein by reference in its entirety for all purposes.

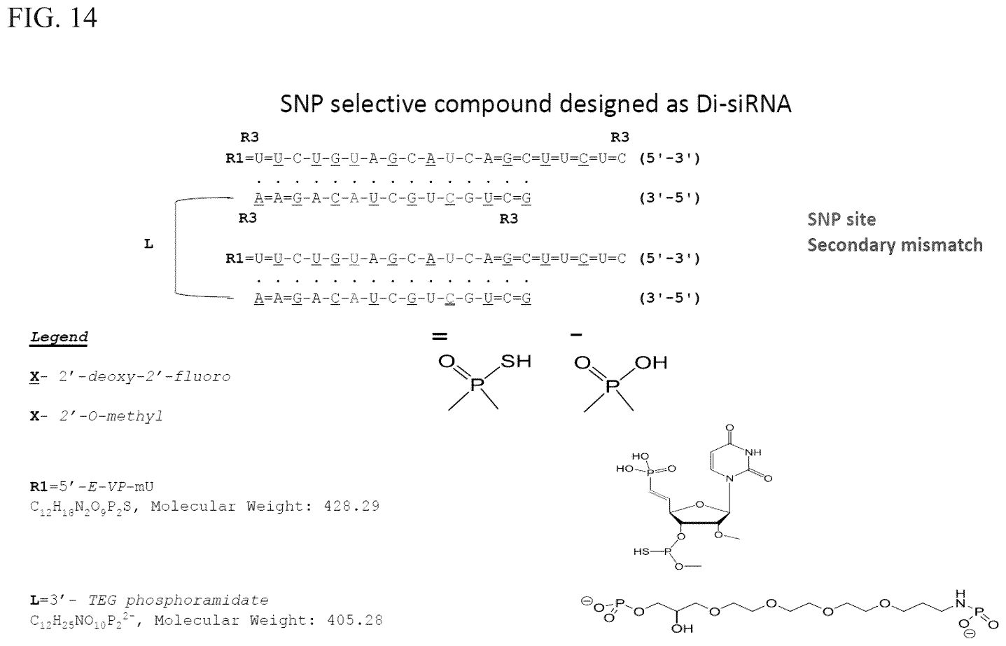

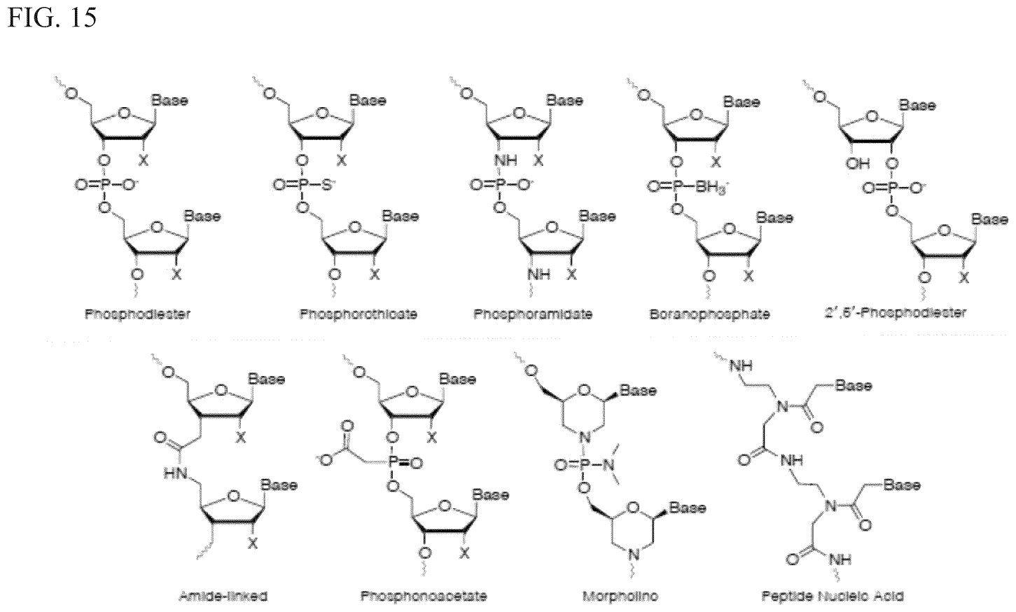

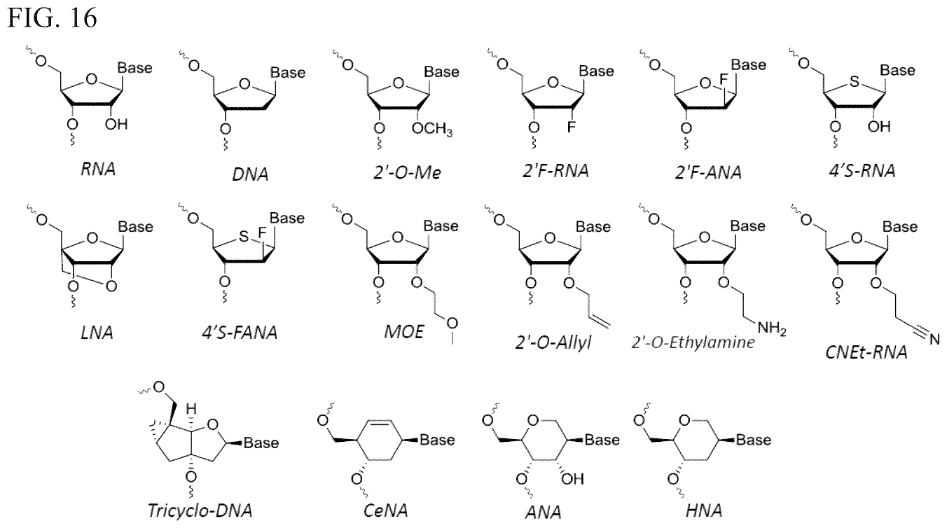

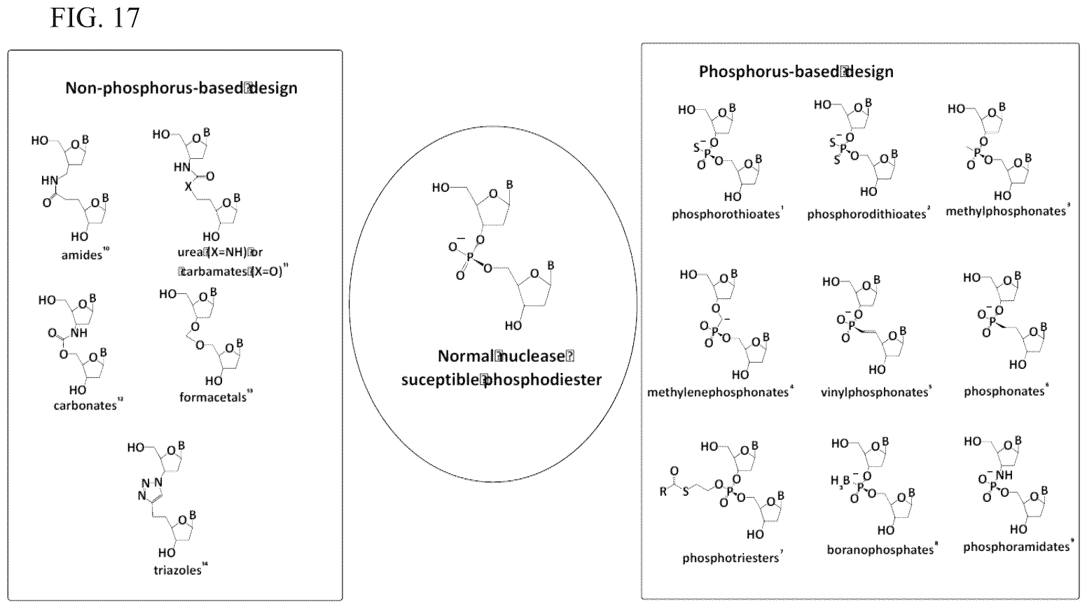

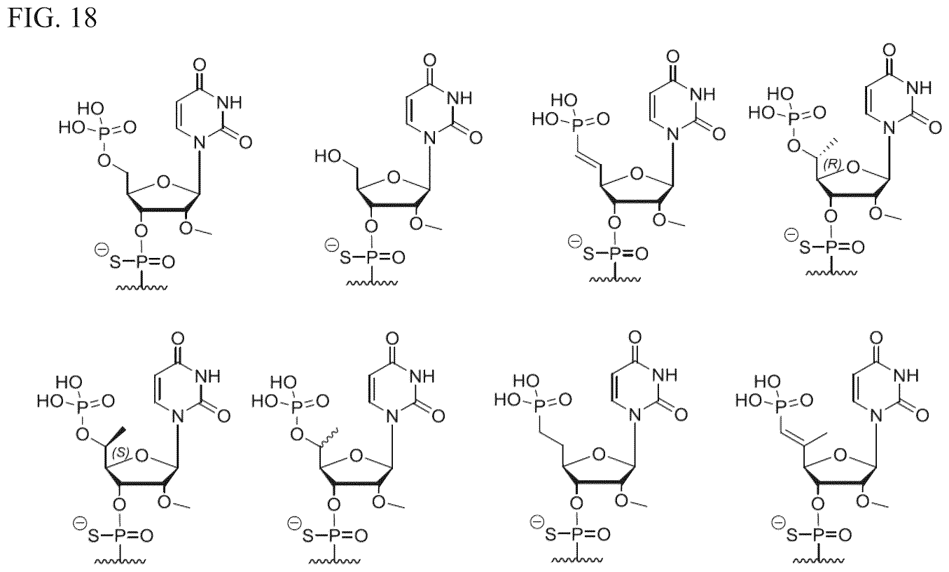

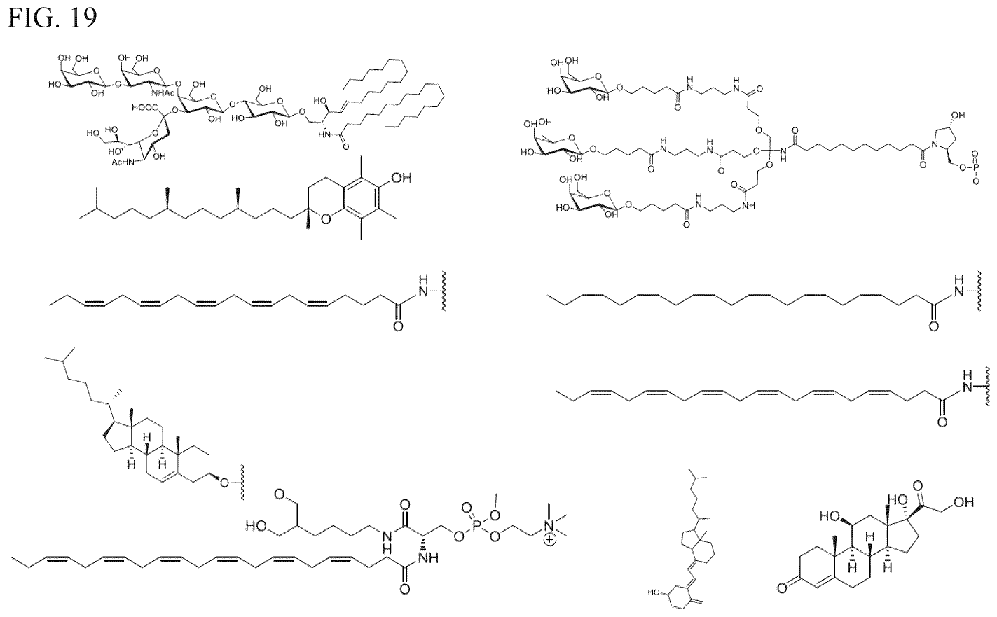

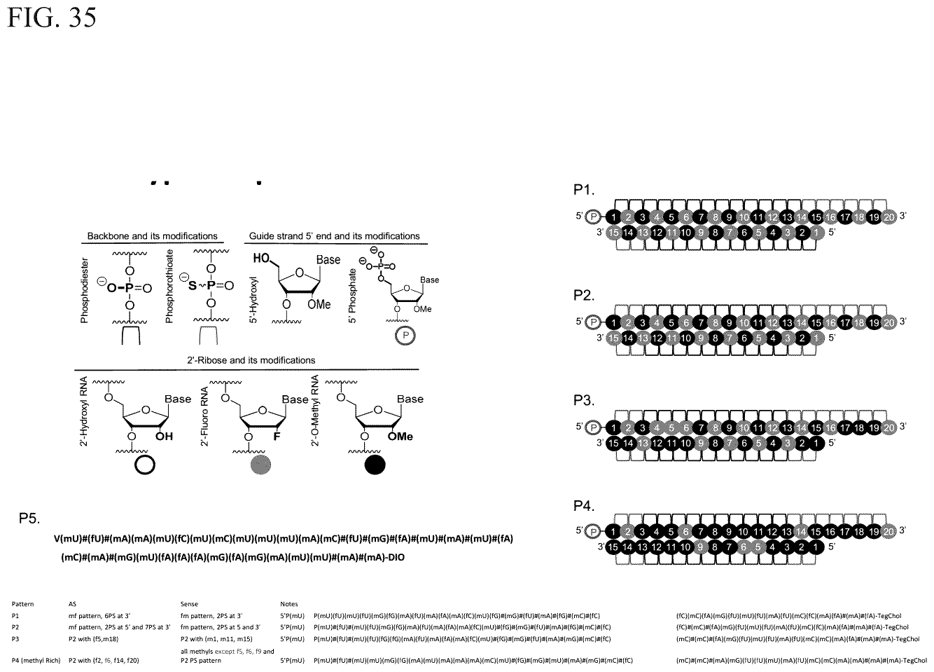

For example, an oligonucleotide described herein may be designed as a di-siRNA (see, e.g., FIG. 14). An oligonucleotide described herein may include one or more different backbone linkages (see, e.g., FIG. 15). An oligonucleotide described herein may include a variety of sugar modifications (see, e.g., FIG. 16). An oligonucleotide described herein may include a variety of internucleotide bonds (see, e.g., FIG. 17). An oligonucleotide described herein may include one or more 5′ stabilization modifications (see, e.g., FIG. 18). An oligonucleotide described herein may include one or more conjugated moieties (see, e.g., FIG. 19). Illustrated in FIG. 35 are a number of exemplary oligonucleotide backbone modifications.

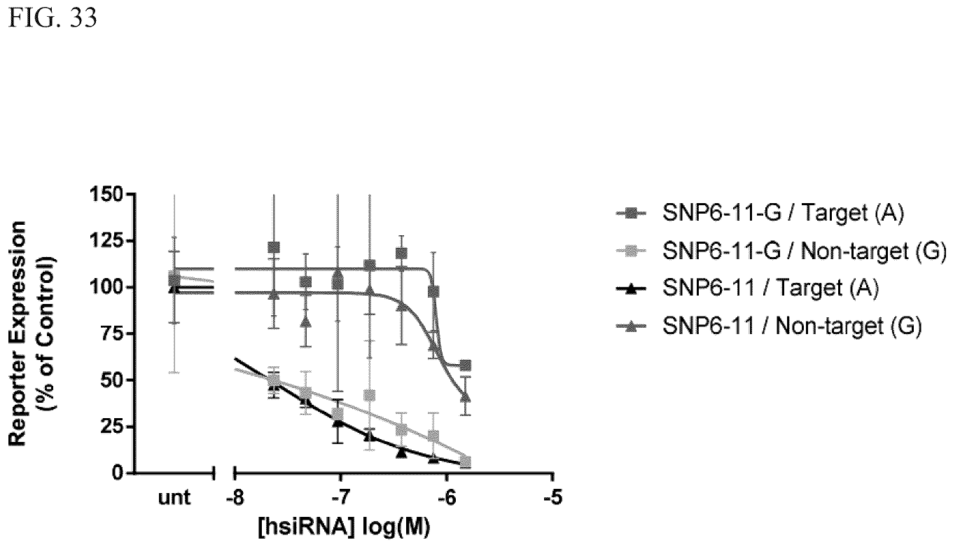

An oligonucleotide described herein can effectively be used to target a G at the SNP site simply by changing the base at the SNP position. As seen in FIG. 33, compound SNP6-11 was synthesized a second time, this time to target a G at the SNP site instead of an A. This allowed for selectively silencing either allele, a strategy that is very useful for patients with different heterozygosities at the same SNP site.

In certain exemplary embodiments, one or more abasic nucleotides are utilized at an SNP position nucleotide, at a MM position nucleotide, at the 5′ end, at the 3′ end, or any combination of these.

In certain exemplary embodiments, hsiRNAs are synthesized with varying sugar modifications around the mismatch to improve allele specificity, e.g., 2′FANA instead of 2′F; triple 2′F or triple 2′OMe around SNP/mismatch position.

### Example 5: HTT Mouse Model

BAC97-HD refer to a transgenic mouse comprising a human bacterial artificial chromosome (BAC) transgenic insert containing the entire pathogenic 170 kb human Huntingtin (htt) genomic locus that was modified by replacing the human htt exon 1 with a loxP-flanked human mutant htt exon 1 sequence containing 97 mixed CAA-CAG repeats encoding a continuous polyglutamine (polyQ) stretch.

Lead compound (SNP6-11) was synthesized into the di-branched chemical scaffold having the structure illustrated in FIG. 31 and subsequently tested in vivo via 40 nmol bilateral intracerebroventricular (ICV) injection (20 nmols to each side) in BAC97-HD female mice at 8 weeks of age. The mice had two copies of normal mouse htt gene with a G at SNP rs362273 and a transgenic insert of pathogenic human htt gene with an A at SNP rs362273A. A nonsense sequence with no target matches in the RNA transcriptome was also synthesized into the same di-branched scaffold and injected in the mice as a negative control (NTC).

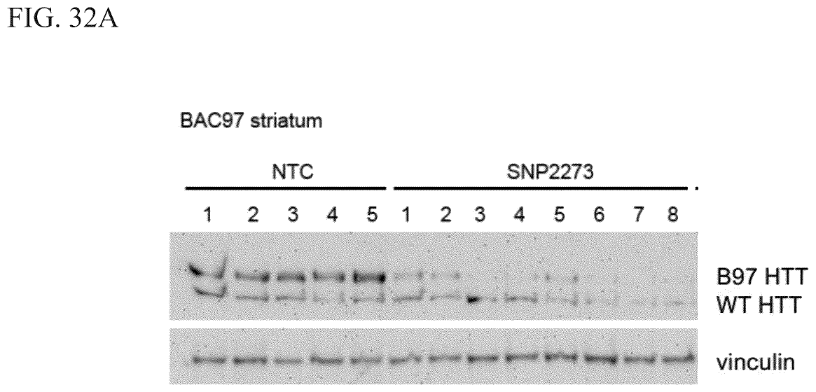

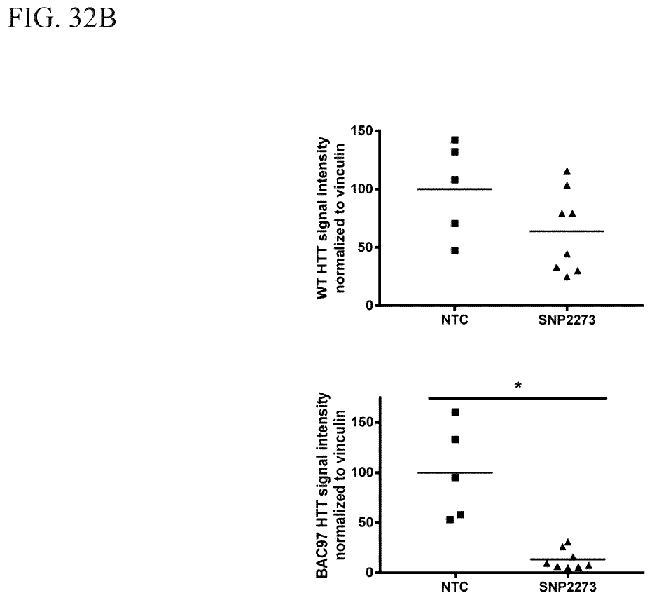

Several brain regions were collected from the mice for RNA and protein analysis 1 month post injection, and HTT protein levels were measured by western blot using Abl antibody. FIG. 32A is a western blot performed on collected striatum tissue, and protein levels normalized to vinculin are presented in FIG. 32B.

### Example 6: SNP Targeting is Sequence-Independent

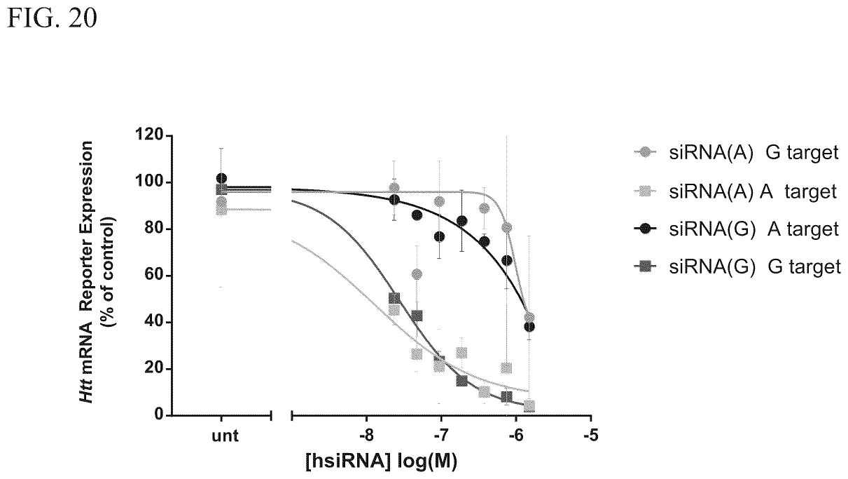

Whether SNP discrimination of lead compounds was sequence-dependent was assessed. Hydrophobically modified RNAs (hsiRNAs) designed to be complimentary to the Huntingtin (htt) mRNA containing a U to G mismatch or a C to A mismatch in rs362273 were used. Both the 6-11 hsiRNA complementary to a U to G mismatch and the 6-11 hsiRNA complementary to a C to A mismatch preferentially cleaved the target SNP (FIG. 20).

### Example 7: Synthesis of Vinyl Phosphonate Modified Intersubunit Linkages

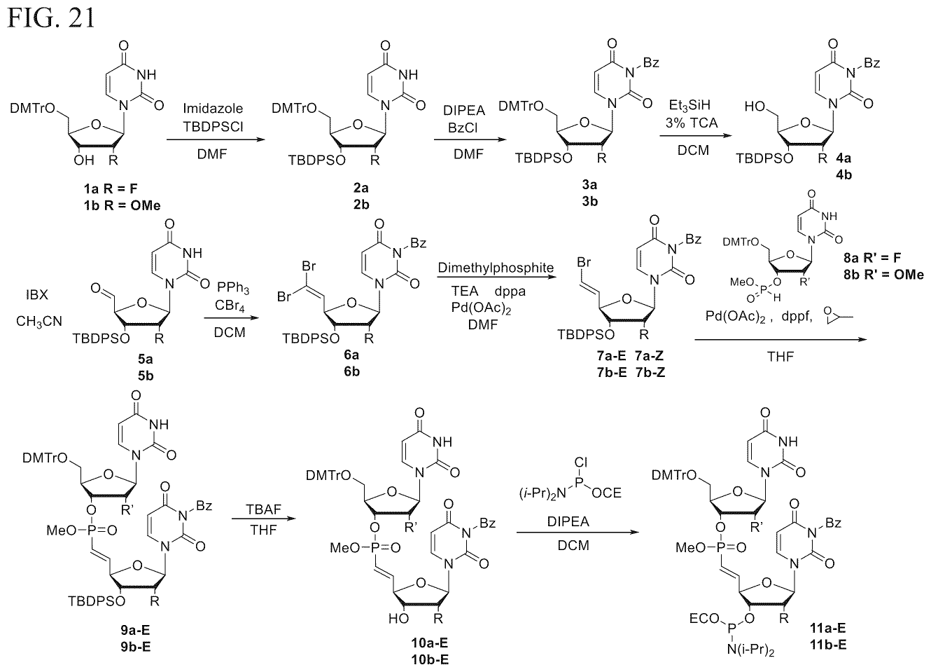

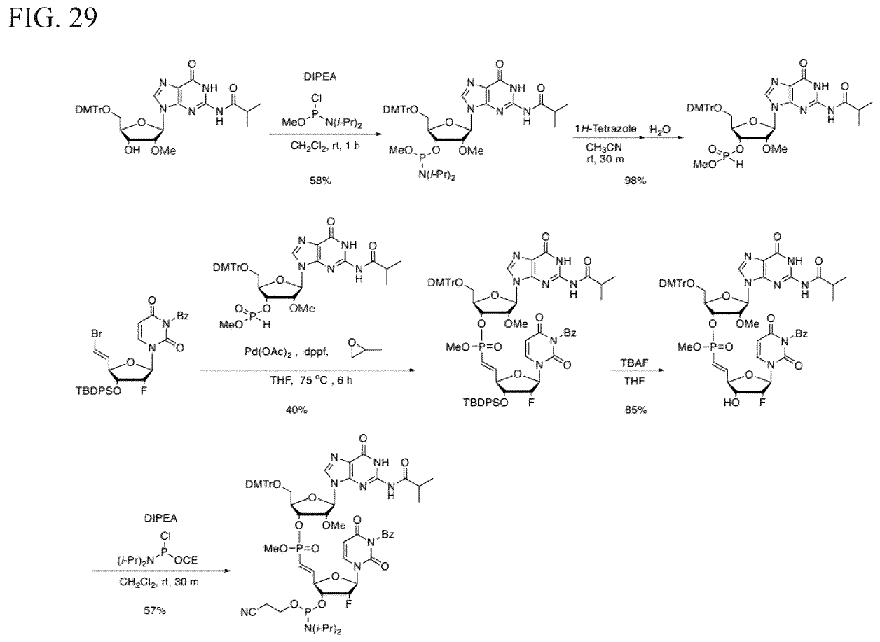

Representative syntheses of the vinyl phosphinate modified intersubunit linkages discussed herein are illustrated in FIGS. 21 and 29. The synthetic procedure of FIG. 21 is detailed below.

### Synthesis of Compound 3a

Anhydrous solution of compound 2a (16.6 g, 20.8 mmol) in pyridine (100 mL) was added anhydrous DIPEA (6.5 mL, 37.4 mmol) and benzoyl chloride (3.6 mL, 31.2 mmol). After the mixture was stirred for 4 hours at room temperature, excess pyridine was evaporated and diluted with CH₂Cl₂. The organic solution was washed by sat. aq. NaHCO₃. The organic layer was collected, dried over MgSO₄, filtered and evaporated. Obtained crude material was purified by silica gel column chromatography (hexane-ethyl acetate, 4:1 to 1:1) yielding compound 3a as a slightly yellow foam (14.5 g, 78%); ¹H NMR (500 MHZ, CDCl₃) δ 7.88-7.87 (m, 2H), 7.84 (d, 1H, J=8.3 Hz), 7.67-7.58 (m, 5H), 7.48-7.45 (m, 4H), 7.39-7.32 (m, 4H), 7.25-7.23 (m, 3H), 7.18-7.17 (m, 2H), 7.12-7.07 (m, 4H), 6.80-6.75 (m, 4H), 6.08 (dd, 1H, J_{HH}=1.5 Hz, J_{HF}=15.2 Hz), 5.14, (d, 1H, J_{HH}=8.3 Hz), 4.59 (ddd, 1H, J_{HH}=3.7, 1.5 Hz, J_{HF}=51.9 Hz), 4.43 (ddd, 1H, J_{HH}=7.4, 4.0 Hz, J_{HF}=19.1 Hz), 4.24-4.23 (m, 1H), 3.79 (s, 6H), 3.62 (dd, 1H, J_{HH}=11.2, 2.0 Hz), 3.35 (dd, 1H, J_{HH}=11.1, 2.0 Hz), 1.00 (s, 9H); ¹³C NMR (126 Hz, CDCl₃) δ 168.4, 161.8, 158.72, 158.66, 148.9, 143.9, 139.4, 135.71. 135.70, 135.1, 134.8, 134.7, 132.3, 132.2, 131.3, 130.4, 130.2, 130.1, 129.1, 128.2, 128.0, 127.91, 127.89, 127.2, 113.19, 113.16, 102.2, 92.5 (d, J_{CF}=194.4 Hz), 87.7 (d, J_{CF}=34.5 Hz), 87.2, 82.4, 70.0 (d, J_{CF}=15.4 Hz), 60.7, 60.4, 55.2, 26.6.

### Synthesis of Compound 4a

Compound 3a (14.5 g, 16.3 mmol) was dissolved into 3% trichloroacetic acid/CH₂Cl₂ solution (200 mL) containing triethylsilane (8.0 mL, 50.1 mmol) and stirred for 1 hour at room temperature. After the solution was washed by sat. aq. NaHCO₃ three times, collected organic layer was dried over MgSO₄, filtered, and evaporated. Obtained crude material was purified by silica gel column chromatography (hexane/ethyl acetate, 4:1 to 3:7) yielding compound 4a as a white foam (8.67 g, 91%); ¹H NMR (500 MHZ, CDCl₃) δ 7.89-7.88 (m, 2H), 7.68-7.64 (6H, m), 7.51-7.45 (m, 4H), 7.42-7.38 (4H, m), 5.93 (dd, 1H, J_{HH}=2.9 Hz, J_{HF}=15.1 Hz), 5.73 (d, 1H, J_{HH}=8.2 Hz), 4.74 (ddd, 1H, J_{HH}=4.1, 3.2 Hz, J_{HF}=52.2 Hz), 4.31 (ddd, 1H, J_{HH}=5.8, 4.7, J_{HF}=15.4 Hz), 4.11-4.09 (m, 1H), 3.82-3.79 (m, 1H), 3.39 (ddd, 1H, J_{HH}=12.1, 5.6, 1.5 Hz), 1.64 (br, 1H), 1.11 (s, 9H); ¹³C NMR (126 Hz, CDCl₃) δ 168.3, 161.8, 149.0, 140.5, 135.7, 135.2, 132.8, 132.3, 131.3, 130.5, 130.4, 130.3, 129.2, 128.02, 127.96, 102.4, 91.8 (d, J_{CF}=91.8 Hz), 89.5 (d, J_{CF}=33.6 Hz), 69.5 (d, J_{CF}=69.5 Hz), 60.3, 26.8.

### Synthesis of Compound 6a

Anhydrous solution of compound 4a (6.5 g, 11.0 mmol) was added IBX (7.7 g, 27.6 mmol) and stirred for 2 hours at 85° C. After cooling the mixture in an ice bath, the precipitate in the solution was filtered off through celite. Collected eluent was evaporated, co-evaporated with anhydrous CH₃CN three times under argon atmosphere, and obtained compound 5a as a white foam was used without further purification. In a separate flask, anhydrous CH₂Cl₂ (25 mL) solution containing CBr₄ (7.3 g, 22.1 mmol) was added PPh₃ (11.6 g, 44.2 mmol) at 0° C. and stirred for 0.5 h at 0° C. To this solution, anhydrous CH₂Cl₂ solution (25 mL) of compound 5a was added dropwise (10 min) at 0° C. and stirred for 2 h at 0° C. After diluting with CH₂Cl₂, the organic solution was washed by aq. sat. NH₄Cl, dried over MgSO₄, filtered, and evaporated. Obtained material was dissolved into minimum amount of diethyl ether and added dropwise to excess diethyl ether solution under vigorously stirring at 0° C. Precipitate in solution was filtered off through celite and eluents was evaporated. Obtained crude material was purified by silica gel column chromatography (hexane/ethyl acetate, 9:1 to 1:1) yielding compound 6a as a white foam (4.3 g, 52%). ¹H NMR (500 MHZ, CDCl₃) δ 7.68-7.84 (m, 2H), 7.70-7.65 (m, 3H), 7.60-7.58 (m, 2H), 7.52-7.49 (m, 2H), 7.42-7.36 (m, 4H), 7.31-7.28 (m, 2H), 7.09 (d, 1H, J=8.2 Hz), 6.25 (d, 1H, J=8.9 Hz), 5.75 (dd, 1H, J_{HF}=8.24 Hz), 5.49 (dd, 1H, J_{HF}=21.4 Hz), 4.77 (t, 1H, J_{HH}=8.5 Hz, J_{HF}=8.5 Hz), 4.38 (dd, 1H, J_{HH}=4.1 Hz, J_{HF}=52.1 Hz), 4.25 (ddd, 1H, J_{HH}=8.1, 4.9 Hz, J_{HF}=19.4 Hz), 1.10 (s, 9H); ¹³C NMR (126 Hz, CDCl₃) δ 167.9, 161.6, 148.3, 141.4, 135.8, 134.7 (d, J_{C-Br}=139.0 Hz), 132.5, 132.2, 131.1, 130.5, 130.3, 130.2, 129.2, 127.9, 102.7, 97.3, 93.3 (d, J_{CF}=39.1 Hz), 91.5 (d, J_{CF}=190.7 Hz), 82.4, 73.9 (d, J_{CF}=16.4 Hz), 26.7.

### Synthesis of Compound 7a-E and 7a-Z

Anhydrous solution of compound 6a (4.2 g, 5.66 mmol) in DMF (25 mL) was added dimethylphosphite (2.09 mL, 22.6 mmol) and triethylamine (1.58 mL, 11.3 mmol) at 0° C., and then stirred overnight at room temperature. After the solution was diluted with ethyl acetate, the organic solution was washed with aq. sat. NH₄Cl and brine. Then the organic solution was dried over MgSO₄, filtered and evaporated. Obtained crude material was purified repeatedly by silica gel column chromatography (hexane/ethyl acetate, 9:1 to 1:1) until all pure isomeric compound were collected separately, giving compound 7a-E (1.95 g, 52%); ¹H NMR (500 MHZ, CDCl₃) δ 7.87-7.85 (m, 2H), 7.89-7.85 (m, 3H), 7.61-7.59 (m, 2H), 7.52-7.48 (m, 2H), 7.45-7.32 (m, 6H), 7.08 (d, 1H, J_{HH}=8.2), 6.49 (d, 1H, J_{HH}=13.7), 5.99 (dd, 1H, J_{HH}=13.7 Hz, 8.1 Hz), 5.75 (d, 1H, J_{HH}=8.2), 5.63 (d, 1H, J_{HF}=19.8 Hz), 4.43 (dd, 1H, J_{HF}=52.6 Hz, J_{HH}=4.3 Hz), 4.42 (t, 1H, J_{HH}=8.0 Hz), 4.07 (ddd, J_{HH}=7.8, 4.7 Hz, J_{HF}=19.5 Hz), 1.08 (s, 9H); ¹³C NMR (126 Hz, CDCl₃) δ 148.4, 140.4, 135.8, 135.7, 135.3, 133.3, 132.3, 132.4, 132.1, 131.1, 130.5, 130.4, 130.3, 129.2, 127.95, 127.93, 112.4, 102.7, 91.7 (d, J_{CF}=36.3 Hz), 91.6 (d, J_{CF}=191.6 Hz), 82.8, 73.9 (d, J_{CF}=16.4 Hz), 26.7, 19.1; and 7a-Z (0.58 g, 15%); ¹H NMR (500 MHz, CDCl₃) δ 7.87-7.85 (m, 2H), 7.68-7.65 (m, 3H), 7.61-7.59 (m, 2H), 7.52-7.48 (m, 2H), 7.42-7.39 (m, 2H), 7.34-7.29 (m, 4H), 7.12 (d, 1H, J_{HH}=8.2 Hz), 6.51 (d, 1H, J_{HH}=7.4 Hz), 5.96 (dd, 1H, J_{HH}=8.4 Hz, 7.4 Hz), 5.75 (d, 1H, J_{HH}=8.2 Hz), 5.57 (dd, 1H, J_{HH}=1.2 Hz, J_{HF}=20.6 Hz), 5.04 (dd, 1H, J_{HH}=8.2 Hz), 4.48 (J_{HH}=3.5 Hz, J_{HF}=53.1 Hz), 4.24 (ddd, 1H, J_{HH}=7.8, 4.9 Hz, J_{HF}=18.6 Hz), 1.09 (s, 9H); ¹³C NMR (126 Hz, CDCl₃) δ 168.0, 161.7, 148.4, 141.4, 135.9, 135.8, 135.2, 132.6, 132.5, 131.2, 130.6, 130.5, 130.2, 130.1, 129.2, 127.8, 127.7, 114.5, 102.6, 93.0 (d, J_{CF}=37.2 Hz), 91.6 (d, J_{CF}=191.6 Hz), 80.3, 74.3 (d, J_{CF}=16.4 Hz), 26.7, 19.1.

### Synthesis of Compound 9a

Anhydrous compound 7a-E (1.95 g, 2.94 mmol) and Pd(OAc)₂ (125 mg, 0.59 mmol) and [1,1′-Bis (diphenylphosphino) ferrocene]dichloropalladium (II) (652 mg, 1.18 mmol) were purged with argon, and then dissolved into anhydrous THF (50 mL). After adding propylene oxide (2.06 mL, 29.4 mmol), compound 8a (2.07 g, 3.24 mmol) was added in one portion and stirred at for 4 h at 70° C. After removing solvent under reduced pressure, the crude mixture was purified by silica gel column chromatography (hexane/ethyl acetate, 50:50 to 0:100) and obtained fractions containing compound 9a were further purified by silica gel column chromatography (CH₂Cl₂-MeOH, 0% to 5%) yielding compound 9a as a mixture of diastereo-isomers (2.04 g, 57%); ³¹P NMR (202 MHZ, CDCl₃) δ 18.3.

### Synthesis of Compound 10a

Compound 9a (2.0 g, 1.64 mmol) in anhydrous THF (22.5 mL) was added 1.0 M TBAF-THF (2.5 mL, 2.5 mmol) and stirred at ambient temperature for 30 minutes. After diluting with CH₂Cl₂ (120 mL), the organic layer was washed with brine, dried over MgSO₄, filtered, and then evaporated. Obtained crude material was purified by silica gel column chromatography (1% TEA-CH₂Cl₂/MeOH, 0% to 6%) yielding compound 10a (1.52 g, 94%); ³¹P NMR (202 MHZ, CDCl₃) δ 19.0, 18.7.

### Synthesis of Compound 11a

Compound 10a (589.7 mg, 0.6 mmol) was rendered anhydrous by repeated co-evaporation with anhydrous CH₃CN and then dissolved into anhydrous CH₂Cl₂ (6.0 mL). To this solution N,N-diisopropylethylamine (0.31 mL, 1.8 mmol) and 2-cyanoethyl N,N-diisopropylchlorophosphoramidite (0.16 mL, 0.72 mmol) were added at 0° C. After stirring for 30 min at 0° C., the reaction mixture was diluted with excess CH₂Cl₂. The organic layer was repeatedly washed with aq. sat. NaHCO₃, dried over MgSO₄, filtered, and evaporated. The obtained crude material was purified by silica gel column chromatography (1% TEA-CH₂Cl₂/MeOH, from 100% to 4%) yielding compound 11a as a white foam (570 mg, 80%); ³¹P NMR (202 MHz, CDCl₃) δ 150.3, 151.2, 151.1, 151.0, 18.72, 18.65, 18.55, 18.3.

### Synthesis of Compound 4b

Anhydrous solution of compound 3b (1.35 g, 2.0 mmol) in pyridine (10 mL) was added DIPEA (0.63 mL, 3.6 mmol) and benzoyl chloride (0.35 mL, 3.0 mmol), and stirred for 3 hours at room temperature. After diluting with excess CH₂Cl₂, the organic solution was washed with aq. sat. NaHCO₃ and brine. After drying over MgSO₄, filtered and evaporating, obtained crude material was used for the next reaction without further purification. Obtained crude material containing compound 3b was added 3% trichloroacetic acid in CH₂Cl₂ (25 mL) and triethylsilane (1 mL, 6.26 mmol), and stirred for 1 hour at room temperature. After the reaction mixture was diluted with CH₂Cl₂, the solution was washed with sat. NaHCO₃aq. three times, dried over MgSO₄, filtered, then evaporated. Obtained crude material was purified by silica gel column chromatography (hexane/ethyl acetate, 4:1 to 1:4) yielding pure compound 4b (596.7 mg, 63% in 2 steps); ¹H NMR (500 MHZ, DMSO-d6) δ 8.13 (d, 1H, J_{HH}=8.2 Hz), 7.95 (d, 2H, J_{HH}=7.3 Hz), 7.81 (t, 1H, J_{HH}=7.5 Hz), 7.69-7.68 (m, 2H), 7.64-7.59 (m, 4H), 7.49-7.42 (m, 6H), 5.93 (d, 1H, J_{HH}=4.6 Hz), 5.26 (t, 1H, J_{HH}=4.6 Hz), 4.36 (dd, 1H, J_{HH}=4.6, 4.6 Hz), 4.02-4.00 (m, 1H), 3.65-3.61 (m, 1H), 3.54 (dd, 1H, J_{HH}=4.6, 4.6 Hz), 3.09 (s, 3H), 1.03 (s, 9H); ¹³C NMR (126 Hz, DMSO-d6) 169.8, 162.1, 149.5, 141.3, 136.1, 135.9, 135.8, 133.4, 133.2, 131.5, 130.7, 130.52, 130.48, 130.0, 128.4, 128.3, 102.1, 86.7, 85.6, 82.8, 79.7, 70.8, 60.2, 57.8, 27.2, 19.4; HRMS (ESI) m/z calcd for C₃₃H₃₅N₂O₇Si⁻ [M-H]⁻ m/z 599.2219, found m/z 599.2258.

### Synthesis of Compound 6b

Anhydrous solution of compound 4b (300.4 mg, 0.5 mmol) in CH₃CN (5 mL) was added IBX (350 mg, 1.3 mmol) and stirred for 2 hours at 85° C. After cooling the solution at 0° C., the precipitate was filtered off by celite-filtration. Obtained eluent containing compound 5b was evaporated, rendered anhydrous by repeated co-evaporation with anhydrous CH₃CN, and used for the next reaction without further purification. Separatory prepared anhydrous solution of CBr₄ (331.6 mg, 1.0 mmol) in CH₂Cl₂ (5.0 mL) was added triphenylphosphine (524.6 mg, 2.0 mmol) at 0° C. in one portion and stirred at 0° C. for 30 minutes. To this solution, compound 5b in anhydrous CH₂Cl₂ (1.5 mL) was added dropwise (10 min) at 0° C. and stirred for 2 h at 0° C. The solution was then diluted with CH₂Cl₂ and washed with sat. NaHCO₃aq. and brine. After the organic solution was dried over MgSO₄, filtered and evaporated, obtained crude material was purified by silica gel column chromatography (hexane/ethyl acetate, 9:1 to 4:6) yielding compound 6b (210.9 mg, 56%); ¹H NMR (500 MHZ, CDCl₃) δ 7.88 (d, 2H, J_{HH}=7.3 Hz), 7.70-7.62 (5H, m), 7.51-7.38 (m, 9H), 7.08 (d, 1H, J_{HH}=8.2 Hz), 6.26 (d, 1H, J_{HH}=8.6 Hz), 5.75 (d, 1H, J_{HH}=8.2 Hz), 5.68 (d, 1H, J_{HH}=0.8 Hz), 4.84 (dd, 1H, J_{HH}=8.6 Hz, 8.6 Hz), 3.86 (dd, 1H, J_{HH}=7.5 Hz, 5.0 Hz), 3.30 (s, 3H), 3.18 (br, 1H), 1.11 (s, 9H); ¹³C NMR (126 Hz, CDCl₃) 168.3, 161.7, 148.6, 138.9, 135.9, 135.8, 134.3, 132.6, 132.4, 131.2, 130.5, 130.4, 130.3, 129.2, 128.0, 127.9, 102.4, 97.5, 90.0, 82.44, 82.39, 74.4, 58.2, 26.7, 19.1; HRMS (ESI) m/z calcd for C₃₄H₃₃Br₂N₂O₆Si⁻ [M-H]⁻ m/z 751.0480 [M-H]⁻, found m/z 753.6495.

### Synthesis of 7b-E and 7b-Z

Anhydrous solution of compound 6b (6.11 g, 8.1 mmol) in DMF (35 mL) was added dimethylphosphite (2.97 mL, 34.0 mmol) and triethylamine (2.26 mL, 17.0 mmol) at 0° C., and then stirred overnight at room temperature. After the solution was diluted with ethyl acetate, the organic solution was washed with sat. NH₄Cl aq. and brine. Then the organic solution was dried over MgSO₄, filtered and evaporated, and obtained crude material was purified repeatedly by silica gel column chromatography (hexane/ethyl acetate, 9:1 to 1:1) until all pure isomeric compound were collected separately, giving compound 7b-E (3.0 g, 55%); ¹H NMR (500 MHZ, CDCl₃) δ 7.89-7.87 (m, 2H), 7.70-7.62 (m, 5H), 7.51-7.39 (m, 8H), 7.10 (d, 1H, J_{HH}=8.3 Hz), 6.47 (dd, 1H, J_{HH}=13.6, 0.8 Hz), 6.01 (dd, 1H, J_{HH}=13.6, 7.9 Hz), 5.76-5.74 (m, 2H), 4.51 (dd, 1H, J_{HH}=7.8, 7.8 Hz), 7.36 (dd, 1H, J_{HH}=7.8 Hz, 4.9 Hz), 3.34 (s, 3H), 3.17 (dd, 1H, J_{HH}=4.7, 1.2 Hz), 1.09 (s, 9H); ¹³C NMR (126 Hz, CDCl₃) δ 168.3, 161.7, 148.7, 138.4, 135.9, 135.8, 135.3, 133.8, 132.6, 132.4, 131.2, 130.5, 130.4, 130.3, 129.2, 128.0, 127.9, 112.1, 102.3, 88.9, 82.8, 82.6, 77.2, 74.2, 58.1, 26.8, 19.1; and 7b-Z (1.23 g, 22%); ¹H NMR (500 MHZ, CDCl₃) δ 7.89-7.87 (m, 2H), 7.72-7.70 (m, 2H), 7.68-7.63 (m, 3H), 7.51-7.44 (m, 4H), 7.41-7.37 (m, 4H), 7.16 (d, 1H, J-8.2 Hz), 6.53 (dd, 1H, J_{HH}=7.4, 0.6 Hz), 6.03 (dd, 1H, J_{HH}=8.5, 7.4 Hz), 5.75-5.73 (m, 2H), 5.12 (t, 1H, J_{HH}=8.1 Hz), 3.93 (dd, 1H, J_{HH}=6.9, 5.0 Hz), 3.32 (br, 1H), 3.26 (s, 3H), 1.10 (s, 9H); ¹³C NMR (126 Hz, CDCl₃) δ 168.3, 161.8, 148.7, 139.3, 135.91, 135.85, 135.22, 132.74, 132.71, 131.2, 130.8, 130.5, 130.23, 130.16, 129.2, 127.78, 127.75, 114.6, 102.2, 90.1, 82.4, 80.6, 77.2, 74.8, 58.1, 26.8, 19.2.

### Synthesis of Compound 8b

Anhydrous 5′-O-DMTr-2′-deoxy-2′-fluoro-3′-[methyl-N,N-(diisopropyl)amino] phosphor-amidite (4.26 g, 6.0 mmol) was dissolved in 0.45 M 1H-tetrazole/CH₃CN solution (27 mL, 12 mmol) and stirred for 30 minutes at room temperature. To this solution, H₂O (3.6 mL) was added and stirred for 30 minutes at room temperature. After diluting with ethyl acetate, the organic solution was washed with brine six times, dried over MgSO₄, filtered and then evaporated. Obtained compound 8b with a slight amount of impurity was used for the next reaction without further purification; ³¹P NMR (CDCl₃, 202 MHZ) δ 8.92, 8.28.

### Synthesis of Compound 9b

Anhydrous compound 7b-E (2.84 g, 4.20 mmol) and Pd (OAc) 2 (188.6 mg, 0.84 mmol) and [1,1′-Bis(diphenylphosphino)ferrocene]dichloropalladium (II) (931.4 mg, 1.68 mmol) were purged with argon, and then dissolved into anhydrous THF (50 mL). After adding propylene oxide (2.94 mL, 42.0 mmol), compound 9b (3.16 g, 5.04 mmol) was added in one portion and stirred at for 4 hours at 70° C. After removing solvent under reduced pressure, the crude mixture was purified by silica gel column chromatography (hexane-ethyl acetate, 50:50 to 0:100) and obtained fractions containing compound 9b were further purified by silica gel column chromatography (1% TEA-CH₂Cl₂/MeOH, 0% to 5%) yielding compound 9b as a mixture of diastereoisomers (3.3 g, 64%); ³¹P NMR (202 MHz, CDCl₃) δ 19.31, 18.72.

### Synthesis of Compound 10b

Compound 9b (3.3 g, 2.70 mmol) in anhydrous THF (36.5 mL) was added 1.0 M TBAF-THF (4.05 mL, 4.05 mmol) and stirred at ambient temperature for 30 minutes. After diluting with CH₂Cl₂ (150 mL), the organic layer was washed with brine, dried over MgSO₄, filtered, and then evaporated. Obtained crude material was purified by silica gel column chromatography (1% TEA-CH₂Cl₂/MeOH, 0% to 8%) yielding compound 10b (1.25 g, 47%); ³¹P NMR (202 MHZ, CDCl₃) δ 19.8, 19.1.

### Synthesis of Compound 11b

Compound 10b (393.2 mg, 0.4 mmol) was rendered anhydrous by repeated co-evaporation with anhydrous CH₃CN and then dissolved into anhydrous CH₂Cl₂ (4.0 mL). To this solution N,N-diisopropylethylamine (0.21 mL, 1.2 mmol) N,N- and 2-cyanoethyl diisopropylchlorophosphoramidite (0.11 mL, 0.48 mmol) were added at 0° C. After stirring for 30 min at 0° C., the reaction mixture was diluted with excess CH₂Cl₂. The organic layer was repeatedly washed with aq. sat. NaHCO₃, dried over MgSO₄, filtered, and evaporated. The obtained crude material was purified by silica gel column chromatography (1% TEA-CH₂Cl₂/MeOH, from 100% to 4%) yielding compound 11b as a white foam (319.6 mg, 68%); ³¹P NMR (202 MHZ, CDCl₃) δ 150.7, 150.4, 150.3, 19.9, 19.5, 19.4, 18.8.

### Example 8: Solid Support-Mediated Synthesis of Vinyl Phosphonate-Modified Oligonucleotides

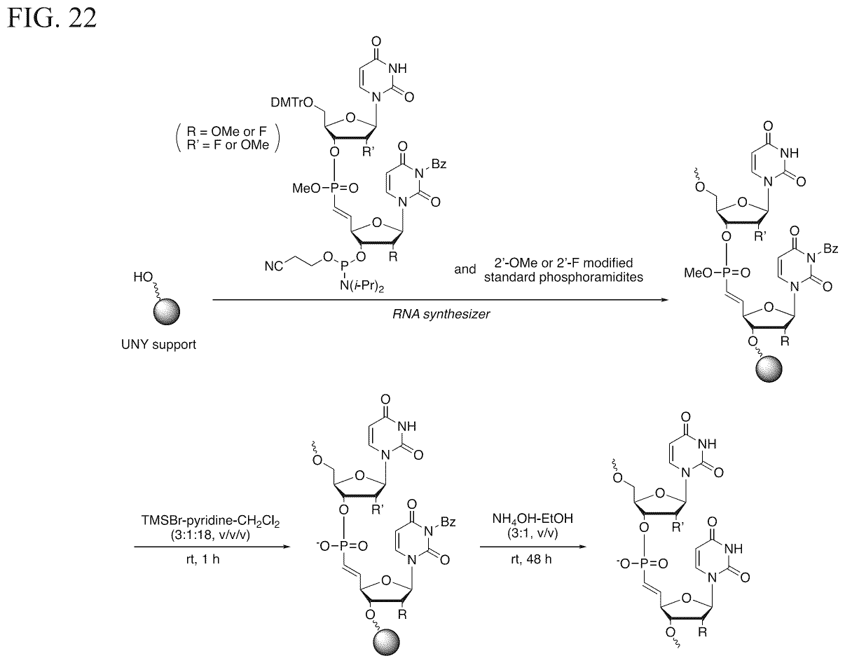

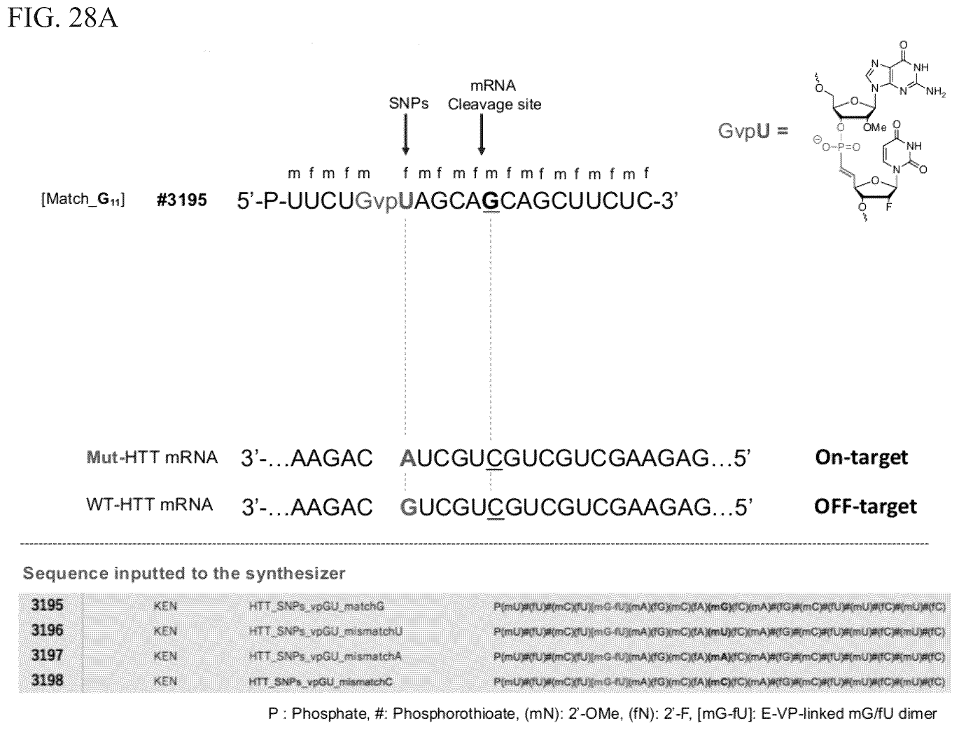

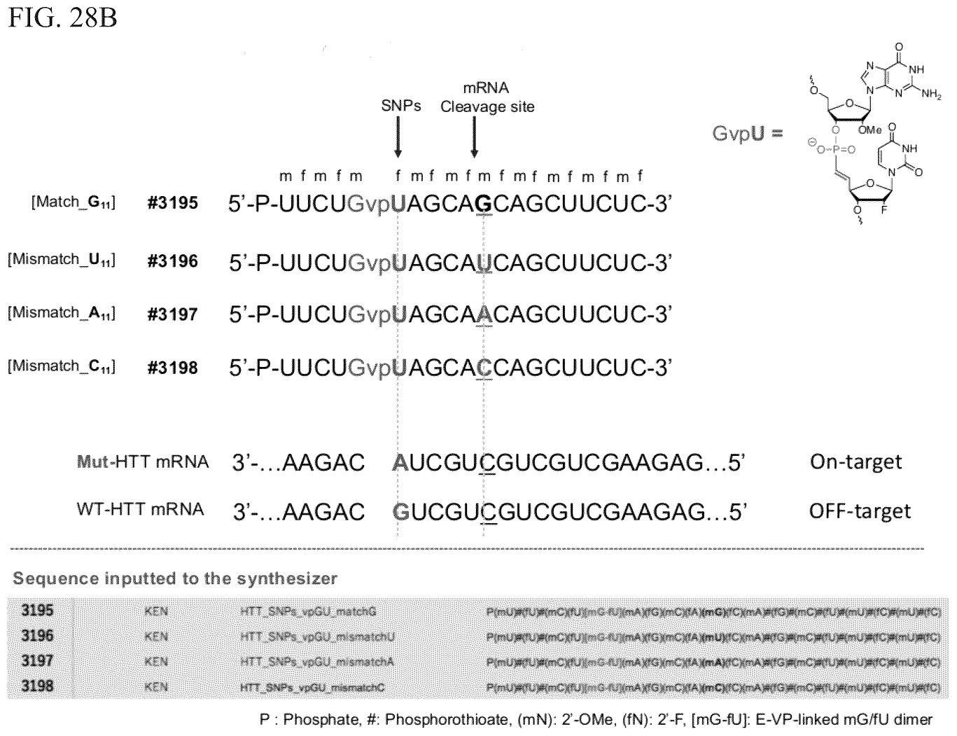

A representative synthesis of an oligonucleotide having a vinyl phosphinate modified intersubunit linkages is illustrated in FIG. 22. Examples of VP-modified sequences that were synthesized can be found in FIGS. 28A and 28B.

Synthesis of Inter-Nucleotide (E)-vinyl phosphonate Modified RNA Oligonucleotides.

The synthesis RNA oligonucleotides having one vinyl phosphonate linkage was performed on MerMade 12 automated RNA synthesizer (BioAutomation) using 0.1 M anhydrous CH₃CN solution of 2′-modified (2′-fluoro, 2′-O-methyl) phosphonamidite and vinylphosphonate-linked dimer phosphoramidites. For the solid support, UnyLinker support (ChemGenes) was used. The synthesis was conducted by standard 1.0 μmol scale RNA phosphoramidite synthesis cycle, which consists of (i) detritylation, (ii) coupling, (iii) capping, and (iv) iodine oxidation. 5-(Benzylthio)-1H-tetrazole in anhydrous CH₃CN was used for phosphoramidite activating reagent, and 3% dichloroacetic acid in CH₂Cl₂ was used for detritylation. 16% N-methylimidazole in tetrehydrofurane (Cap A) and 80:10:10 (v/v/v) tetrhydrofurane-Ac₂O-2,6-lutidine (Cap B) were used for capping reaction. 0.02 M I₂ in THF-pyridine-H₂O (7:2:1, v/v/v) was used for oxidation and 0.1 M 3-[(Dimethylamino-methylidene)amino]-3H-1,2,4-dithiazole3-thione in pyridine:CH₃CN (9:1, v/v) was used for sulfurizing. For 5′-terminal phosphorylation, bis (2-cyanoethyl)-N,N-diisopropyl phosphoramidite was used. For the 3′-cholesterol modified RNA oligonucleotide synthesis, cholesterol 3′-lcaa CPG 500 Å (ChemGenes) was used, and RNA synthesis was conducted in the same condition as the condition used for VP-modified RNAs. After the chemical chain elongation, deprotection and cleavage from the solid support were conducted by NH₄OH-EtOH (3:1, v/v) for 48 hours at 26° C. In the case of vinyl phosphonate modified RNA, RNA on solid support was first treated with TMSBr-pyridine-CH₂Cl₂ (3:1:18, v/v/v) for 1 h at ambient temperature in RNA synthesis column. Solid support was then washed by water (1 mL×3), CH₃CN (1 mL×3) and CH₂Cl₂ (1 mL×3) by flowing solution thorough synthesis column, and then dried under vacuum. After transferring the solid support to screw-capped sample tube, base treatment by NH₄OH-EtOH (3:1, v/v) for 48 h at 26° C. was conducted. Crude RNA oligonucleotide without cholesterol conjugate was purified by standard anion exchange HPLC, whereas RNAs with cholesterol-conjugate were purified by reversed-phase HPLC. Obtained all purified RNAs were desalted by Sephadex G-25 (GE Healthcare) and characterized by electrospray ionization mass spectrometry (ESI-MS) analysis.

### Example 9: Silencing Efficacy

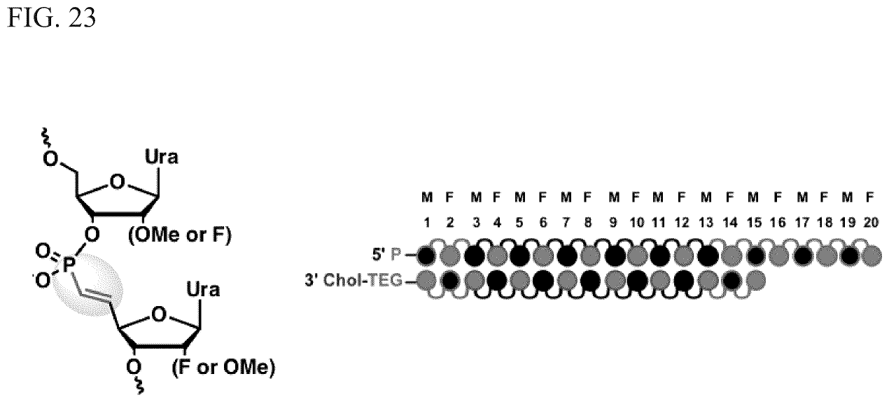

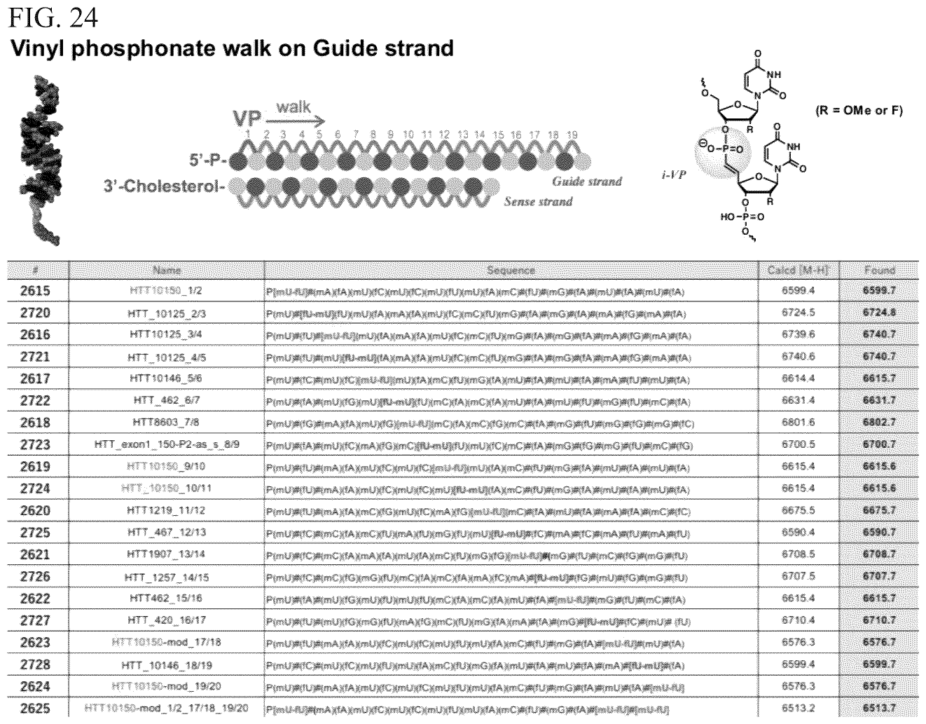

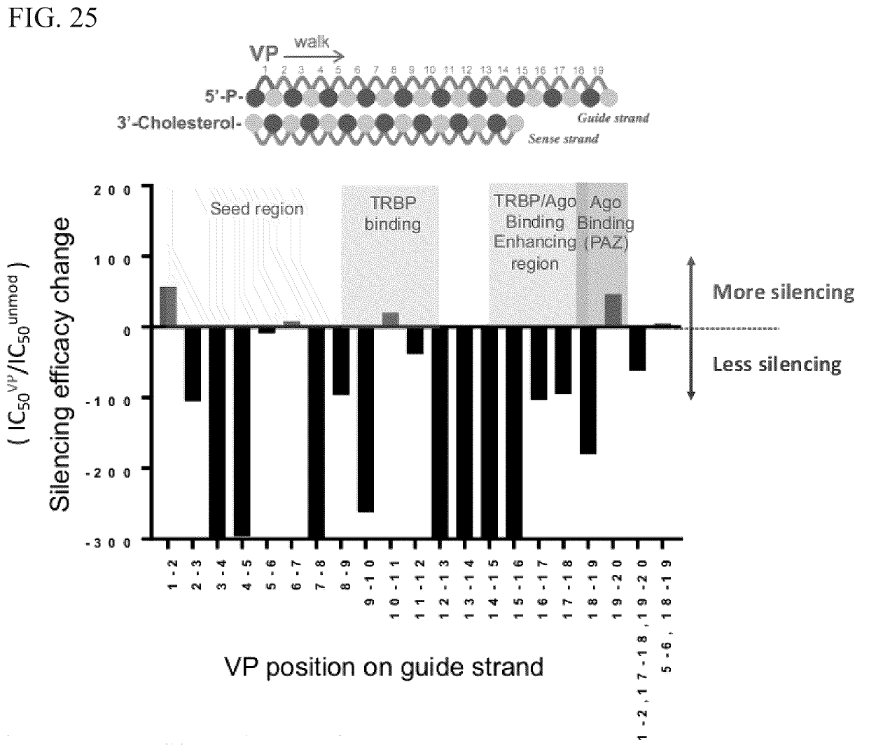

FIGS. 23 and 24 provide visual representations of the VP-modified siRNA studied herein. FIG. 25 exemplifies the effect that one or more vinyl phosphonate modifications in an intersubunit linkage at varying positions on the guide strand has on silencing. As can be seen from the data in FIG. 25, RISC is very sensitive to VP modification, and having a mismatch base pair at various positions can disrupt siRNA potency.

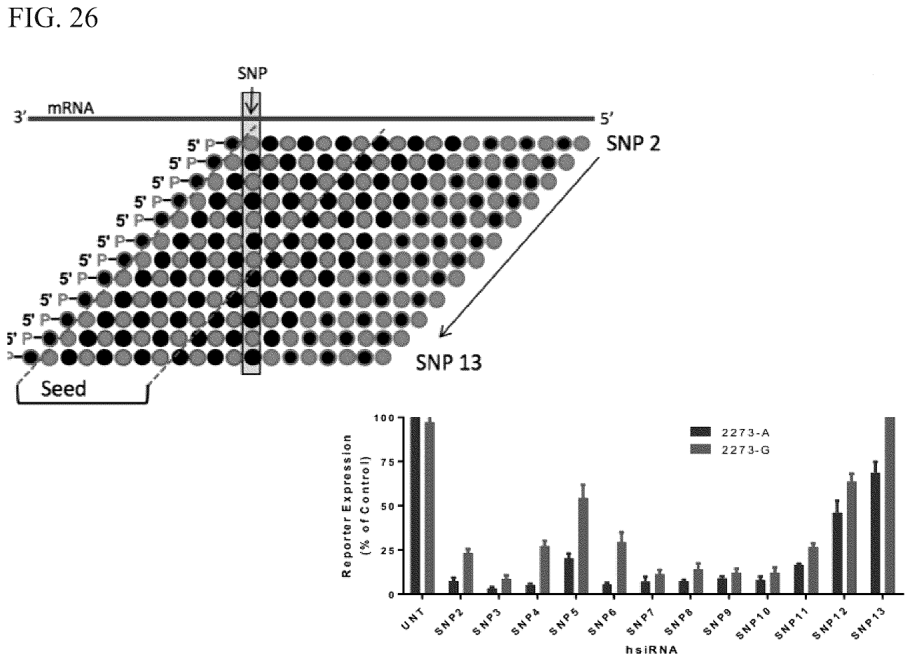

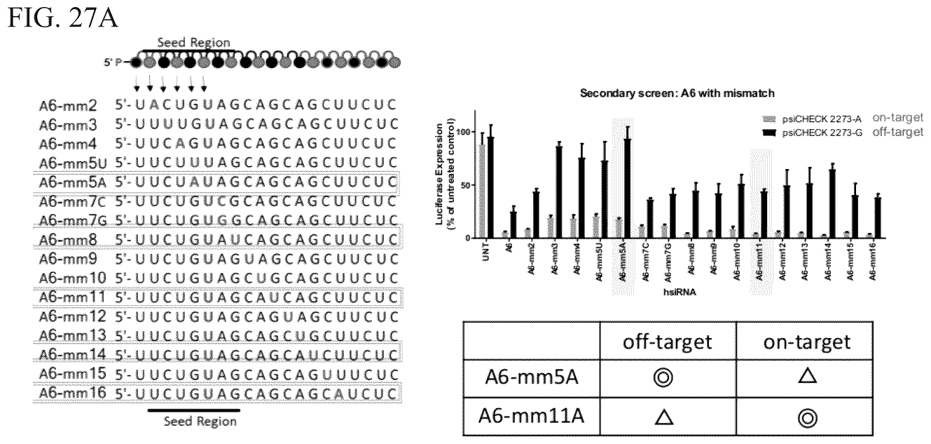

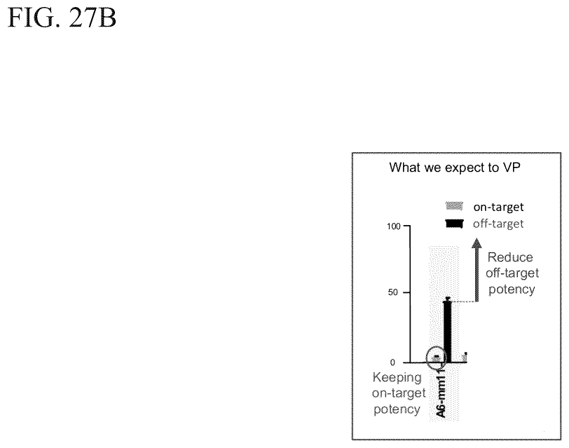

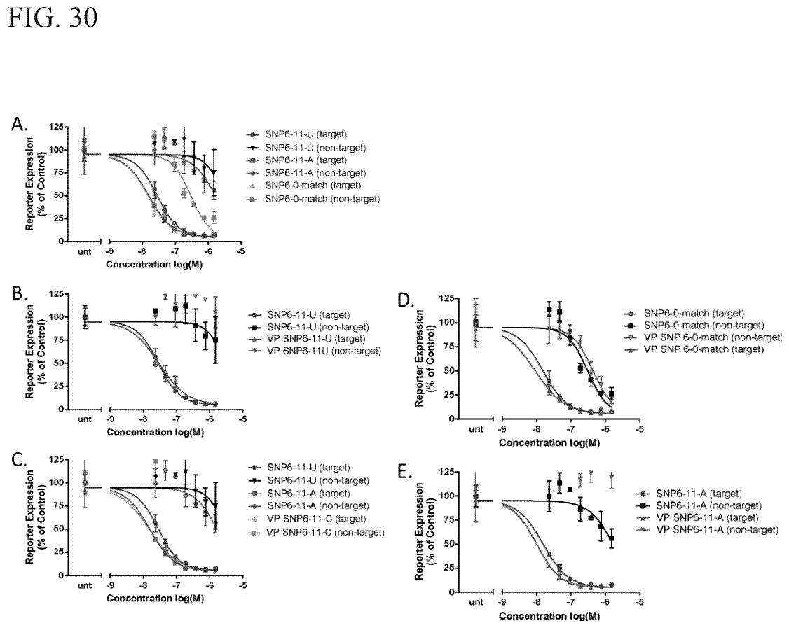

FIGS. 26, 27A, and 27B also illustrate the ability of VP-modified siRNA to silence the mutant allele. As can be seen by FIGS. 27A and 27B, adding a mismatch in the siRNA sequence could improve allelic discrimination without affecting mutant allele silencing. FIG. 30 demonstrates that the introduction of a VP-modified linkage next to the SNP site significantly enhanced target/non-target discrimination of SNP-selective siRNAs. Compounds containing primary (position 6) and secondary (position 11) SNPs were synthesized with or without a VP-modification between positions 5 and 6. As can be seen in FIG. 30, the presence of a VP-modification had no impact on “on target” activity, but fully eliminated any detectable silencing for non-target mRNAs. The method for generating the data in FIGS. 25, 26, 27A, and 27B is described below.

hsiRNA Passive Delivery.

Cells were plated in Dulbecco's Modified Eagle's Medium containing 6% FBS at 8,000 cells per well in 96-well cell culture plates. hsiRNAs were diluted to twice the final concentration in OptiMEM (Carlsbad, CA; 31985-088), and 50 μL diluted hsiRNAs were added to 50 μL of cells, resulting in 3% FBS final. Cells were incubated for 72 hours at 37° C. and 5% CO₂. The maximal dose in the in vitro dose response assays was 1.5 μM compound.

Method for Quantitative Analysis of Target mRNA.

mRNA was quantified from cells using the QuantiGene 2.0 assay kit (Affymetrix, QS0011). Cells were lysed in 250 μL diluted lysis mixture composed of one part lysis mixture (Affymetrix, 13228), two parts H₂O and 0.167 μg/μL proteinase K (Affymetrix, QS0103) for 30 min at 55° C. Cell lysates were mixed thoroughly, and 40 μL of each lysate was added per well of a capture plate with 20 μL diluted lysis mixture without proteinase K. Probe sets for human HTT and HPRT (Affymetrix; #SA-50339, SA-10030) were diluted and used according to the manufacturer's recommended protocol. Datasets were normalized to HPRT.

### Method for Creating Bar Graph.

Data were analyzed using GraphPad Prism 7 software (GraphPad Software, Inc., San Diego, CA). Concentration-dependent IC₅₀ curves were fitted using a log (inhibitor) versus response—variable slope (four parameters). For each cell treatment plate, the level of knockdown at each dose was normalized to the mean of the control group (untreated group). The lower limit of the curve was set to less than 5, and the upper limit of the curve was set to greater than 95. To create the bar graph, the percent difference was calculated by subtracting the IC₅₀ value for each compound from the IC₅₀ value for each corresponding control compound, dividing by the IC₅₀ value for the control compound, and multiplying by 100. If the percent difference was less than −500%, the percent difference was artificially set to −500%. The lower limit of the graph was cut at −300%.

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

### Example 10: Primary Screen Yields Multiple Efficacious siRNA Sequences for SNP rs362307 Heterozygosity

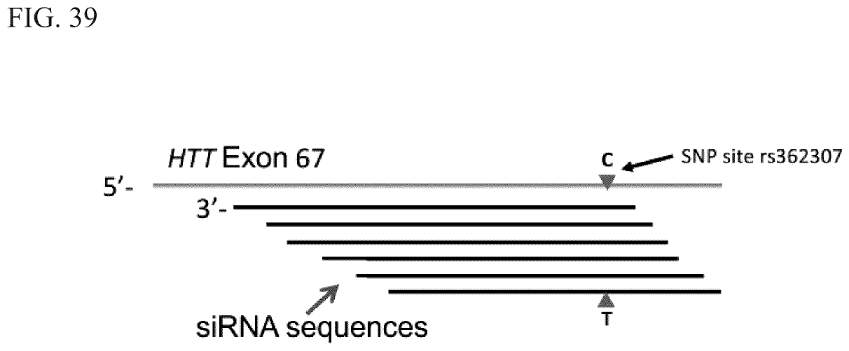

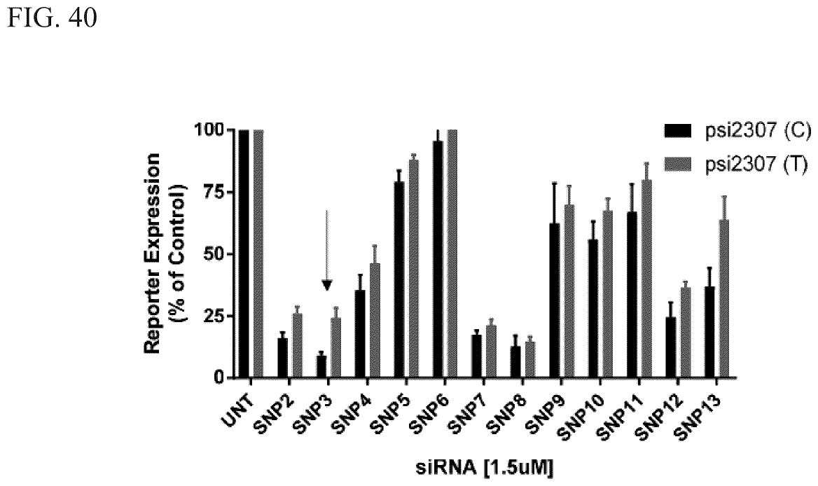

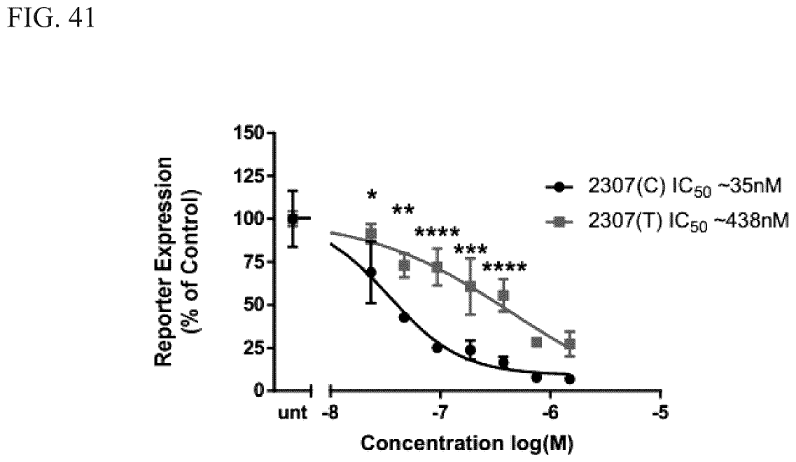

siRNAs designed to be complimentary to the HTT mRNA containing an alternative mutant SNP (rs362307) (FIG. 39) were all screened with reporter plasmids containing the target region for the SNP of interest (FIG. 40). HeLa cells transfected with one of two reporter plasmids were reverse transfected with 1.5 μM hsiRNAs by passive uptake, and treated for 72 hours. The number following SNP represents the position of the SNP in the siRNA. It was expected that this SNP would be more difficult to target based on the high G/C content of the region around it. It appears that placing the SNP in position 3 provided the most SNP discrimination, without losing efficacy against the mutant allele, showing that the best SNP position is sequence-specific (FIG. 41). This primary screening process may thus be carried out for selecting the best SNP position for any SNP.

### Example 11: When Applied to SNP rs362307, a Secondary Mismatch Continues to Improve Allelic Discrimination

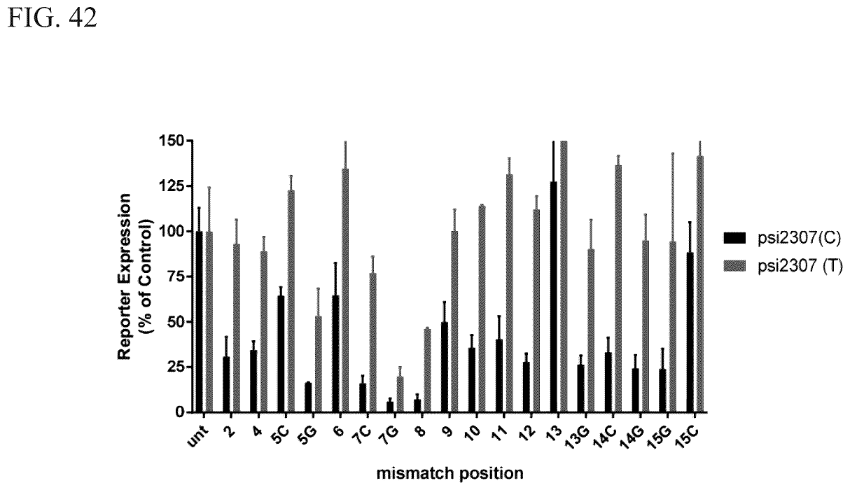

As reported in FIG. 42, primary screen of new sequences with mismatches introduced into all possible positions yields multiple efficacious hsiRNAs with increased SNP discrimination at position rs362307 as well. Introducing a mismatch at position 7 and 8 appeared to increase selectivity while preserving target silencing efficacy. Other secondary mismatches provided excellent discrimination, but less activity overall.

### Example 12: Measuring SNP Discrimination in Sequences Including an SNP

To measure SNP discrimination by each of the sequences disclosed in Tables 5-7 (i.e., each hsiRNA having a particular SNP position nucleotide and mismatch (MM) position nucleotide combination), psiCHECK reporter plasmids containing either a wild-type region of htt or the same region of htt with the SNP of the sequence are prepared and tested using a dual-luciferase. HeLa cells transfected with one of two reporter plasmids are reverse transfected with hsiRNAs by passive uptake, and treated for 72 hours. Luciferase activities are measured in the assays with or without the additional mismatch, and are then plotted in dose response curves and compared to reveal sequences yielding the best results in terms of discrimination and efficacy of silencing.

### Example 13: Synthesis of a Phosphinate-Modified Intersubunit Linkage

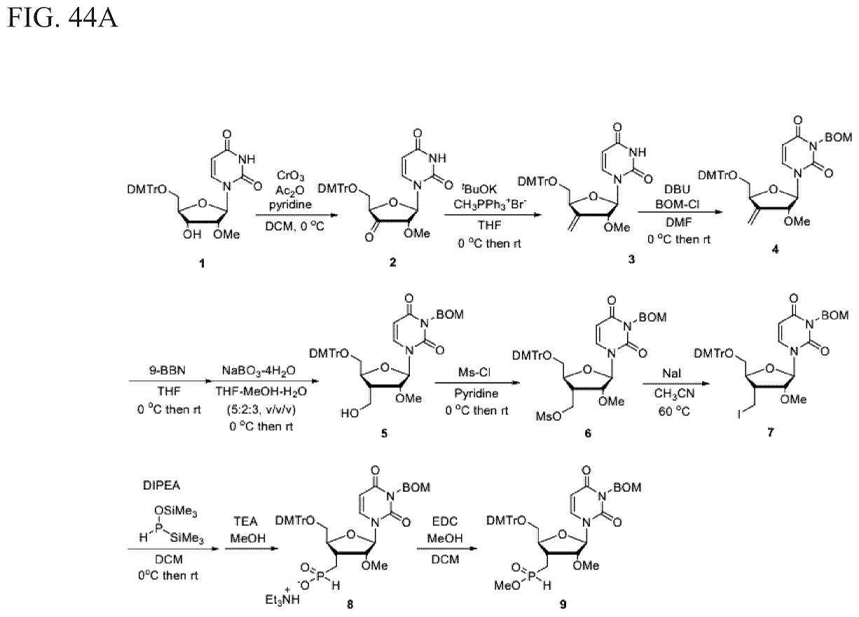

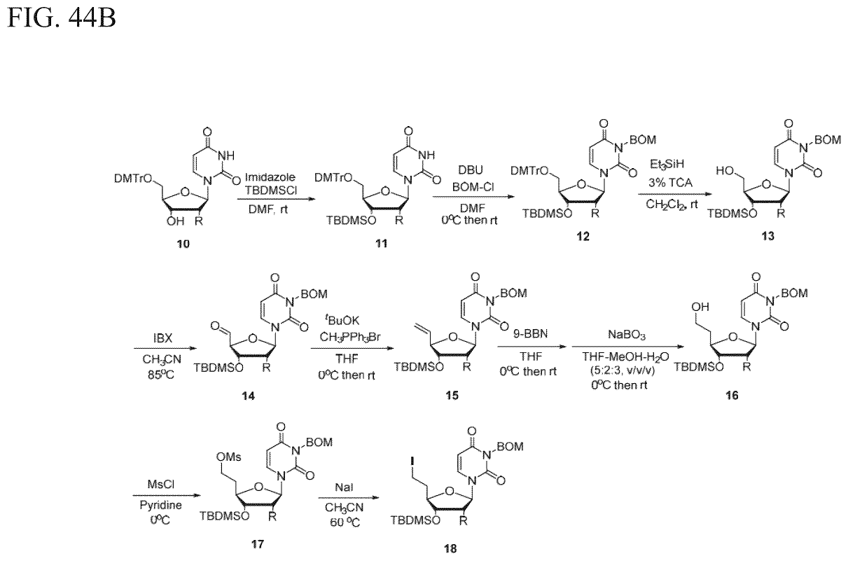

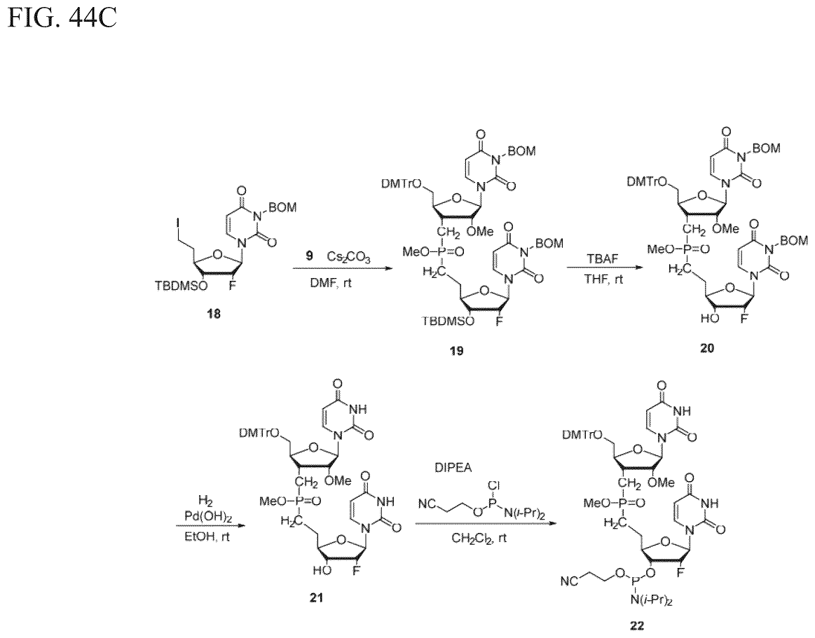

A method for preparing a phosphinate-modified intersubunit linkage is summarized in FIGS. 44A-44C. This method involves Jones oxidation from a free alcohol to the corresponding ketone followed by a Wittig olefination to achieve the exomethylene moiety shown in intermediate compound 3. Protecting of the amide with BOM followed by hydroboration-oxidation results in the free alcohol intermediate 5. Mesylation followed by a modified Finkelstein reaction produces the iodinated intermediate 7, which then undergoes further functionalization to achieve the methyl phosphinate monomer 9.

To achieve monomer 18, various protection and deprotection steps are employed to achieve intermediate 13. IBX oxidation produces the corresponding ketone followed by Wittig olefination to access the methylene. Once again, hydroboration-oxidation followed by mesylation and Finkelstein reaction results in monomer 18.

Combining monomers 9 and 18 under basic conditions produces phosphinate-linked dimer 19. Acid-mediated and Pearlman's catalyzed deprotection followed by further phosphanamine functionalization results in dimer 22.

### Example 14: Altering 2′-OMe/2′-F Content to Modify Efficacy and Discrimination

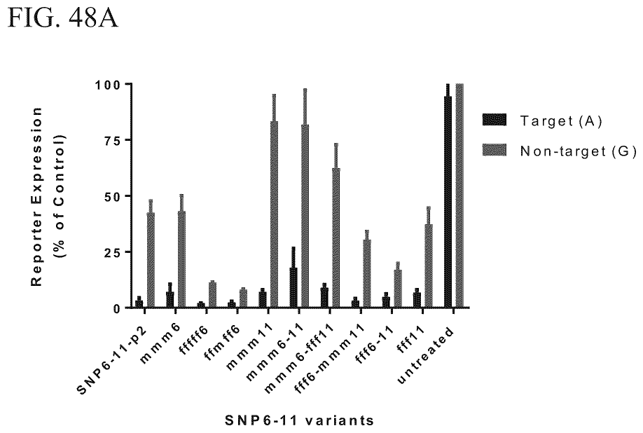

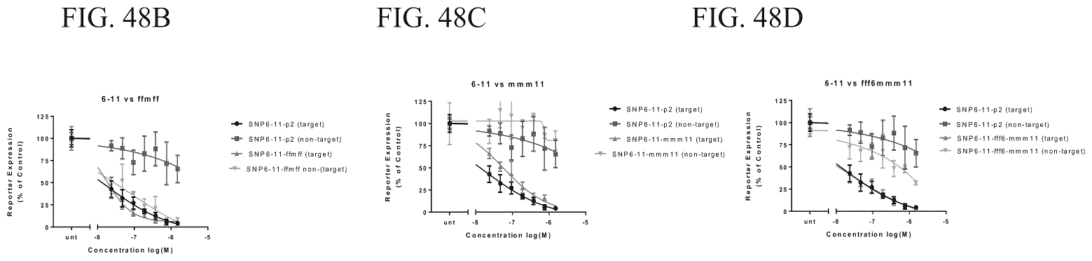

By altering the 2′-O-methyl/fluoro backbone modification pattern around the SNP and mismatch site, efficacy and discrimination of the siRNA was modified (FIG. 48A-48D). Heavy 2′-fluorination adjacent to the SNP position improved target binding, but decreased target discrimination. Subsequently adding heavy 2′-O-methylation around the mismatch rescued discrimination lost due to fluorination. Although the original chemical modification pattern described supra was beneficial for in vivo study, the technique described in this example can be used to fine-tune SNP-targeting compounds described herein, and to identify additional new SNP-targeting compounds.

1-11. (canceled)

12. A nucleic acid comprising:
(a) a 5′ end and a 3′ end;
(b) a seed region that is complementary to a region of a gene comprising an allelic polymorphism;
(c) a single nucleotide polymorphism (SNP) position nucleotide at a position within the seed region, wherein the SNP position nucleotide is complementary to the allelic polymorphism;
(d) a mismatch (MM) position nucleotide that is a mismatch with a nucleotide in the gene; and
(e) at least one modified nucleotide (X) on either side of the SNP position nucleotide, wherein each X is located within four, three, or two nucleotides from the SNP position nucleotide; and/or at least one modified nucleotide (Y) on either side of the MM position nucleotide, wherein each Y is located within four, three, or two nucleotides from the MM position nucleotide.

13. The nucleic acid of claim 12, wherein X comprises a sugar modification selected from the group consisting of 2′-O-methyl (2′-OMe), 2′-fluoro (2′-F), 2′-ribo, 2′-deoxyribo, 2′-F-4′-thioarabino (2′-F-ANA), 2′-O-(2-methoxyethyl) (2′-MOE), 4′-S-RNA, locked nucleic acid (LNA), 4′-S-F-ANA, 2′-O-allyl, 2′-O-ethylamine, 2′-O-cyanoethyl-RNA (CNet-RNA), tricyclo-DNA, cyclohexenyl nucleic acid (CeNA), arabino nucleic acid (ANA), and hexitol nucleic acid (HNA).

14. The nucleic acid of claim 12, wherein Y comprises a sugar modification selected from the group consisting of 2′-OMe, 2′-F, 2′-ribo, 2′-deoxyribo, 2′-F-ANA, 2′-MOE, 4′-S-RNA, LNA, 4′-S-F-ANA, 2′-O-allyl, 2′-O-ethylamine, CNet-RNA, tricyclo-DNA, CeNA, ANA, and HNA.

15. The nucleic acid of claim 12, wherein an X is positioned immediately 5′ to the SNP position nucleotide and/or immediately 3′ to the SNP position nucleotide.

16. (canceled)

17. The nucleic acid of claim 12, wherein a Y is positioned immediately 5′ to the MM position nucleotide and/or immediately 3′ to the MM position nucleotide.

18. (canceled)

19. The nucleic acid of claim 12, wherein the SNP position nucleotide is present from position 2 to position 6 from the 5′ end.

20. The nucleic acid of claim 12, wherein the MM position nucleotide is located 2-11 nucleotides from the SNP position nucleotide.

21. (canceled)

22. The nucleic acid of claim 12, wherein X and Y comprise identical nucleotide modifications.

23. The nucleic acid of claim 12, wherein X and Y comprise different nucleotide modifications.

24. A nucleic acid comprising:
(a) a 5′ end and a 3′ end;
(b) a seed region that is complementary to a region of a gene comprising an allelic polymorphism;
(c) a single nucleotide polymorphism (SNP) position nucleotide that is complementary to the allelic polymorphism;
(d) a mismatch (MM) position nucleotide that is a mismatch with a nucleotide in the gene; and
(e) at least one 2′-fluoro-ribonucleotide on either side of the SNP position nucleotide, wherein each 2′-fluoro-ribonucleotide is located within four, three, or two nucleotides from the SNP position nucleotide; and
(f) at least one 2′-methoxy-ribonucleotide on either side of the MM position nucleotide, wherein each 2′-methoxy-ribonucleotide is located within four, three, or two nucleotides from the MM position nucleotide; or
(g) at least three 2′-fluoro-ribonucleotides located within four, three, or two nucleotides from the SNP position nucleotide; and
(h) at least three 2′-methoxy-ribonucleotides located within four, three, or two nucleotides from the MM position nucleotide.

25. The nucleic acid of claim 24, wherein a 2′-fluoro-ribonucleotide is positioned immediately 5′ to the SNP position nucleotide and/or a 2′-fluoro-ribonucleotide is positioned immediately 3′ to the SNP position nucleotide.

26. (canceled)

27. The nucleic acid of claim 24, wherein a 2′-methoxy-ribonucleotide is positioned immediately 5′ to the MM position nucleotide and/or a 2′-methoxy-ribonucleotide is positioned immediately 3′ to the MM position nucleotide.

28. (canceled)

29. The nucleic acid of claim 24, wherein the SNP position nucleotide is present in a seed region, and wherein the MM position nucleotide is located 2-11 nucleotides from the SNP position nucleotide.

30. The nucleic acid of claim 29, wherein the SNP position nucleotide is present from position 2 to position 6 from the 5′ end, and wherein the MM position nucleotide is located 2-6 nucleotides from the SNP position nucleotide.

31-39. (canceled)

40. An siRNA molecule comprising a sense strand having complementarity to a target gene and an antisense strand having complementarity to the sense strand, wherein the antisense strand comprises the nucleic acid of claim 12.

41-44. (canceled)

45. A branched oligonucleotide comprising two or more siRNA molecules covalently bound to one another, wherein each siRNA molecule is, independently, an siRNA molecule of claim 40.

46-47. (canceled)

48. A double-stranded nucleic acid comprising:
(a) a first strand of nucleotides comprising:
(i) a 5′ end and a 3′ end;
(ii) a seed region that is complementary to a region of a gene comprising an allelic polymorphism;
(iii) a single nucleotide polymorphism (SNP) position nucleotide at a position within the seed region, wherein the SNP position nucleotide is complementary to the allelic polymorphism;
(iv) a mismatch (MM) position nucleotide that is not complementary to a nucleotide in the gene; and
(v) at least one modified nucleotide located on either side of the SNP position nucleotide, on either side of the MM position nucleotide, or a combination thereof; wherein each modified nucleotide is located within four, three, or two nucleotides from the SNP position nucleotide or from the MM position nucleotide, respectively;
(b) a second strand of nucleotides that is complementary to the first strand of nucleotides.

49. The double-stranded nucleic acid of claim 48, wherein the modified nucleotide comprises a modification selected from the group consisting of 2′-O-methyl (2′-OMe), 2′-fluoro (2′-F), 2′-ribo, 2′-deoxyribo, 2′-F-4′-thioarabino (2′-F-ANA), 2′-O-(2-methoxyethyl) (2′-MOE), 4′-S-RNA, locked nucleic acid (LNA), 4′-S-F-ANA, 2′-O-allyl, 2′-O-ethylamine, 2′-O-cyanoethyl-RNA (CNet-RNA), tricyclo-DNA, cyclohexenyl nucleic acid (CeNA), arabino nucleic acid (ANA), hexitol nucleic acid (HNA), and a combination thereof.

50-60. (canceled)

61. A branched oligonucleotide comprising two or more siRNA molecules covalently bound to one another, wherein each siRNA molecule comprises a double-stranded nucleic acid comprising:
(a) a first strand of nucleotides comprising:
(i) a 5′ end, a 3′ end;
(ii) a seed region that is complementary to a region of a gene comprising an allelic polymorphism;
(iii) a single nucleotide polymorphism (SNP) position nucleotide at a position within the seed region, wherein the SNP position nucleotide is complementary to the allelic polymorphism;
(iv) a mismatch (MM) position nucleotide that not complementary to a nucleotide in the gene; and
(v) at least one modified nucleotide located on either side of the SNP position nucleotide, on either side of the MM position nucleotide, or a combination thereof; wherein each modified nucleotide is located within four, three, or two nucleotides from the SNP position nucleotide or from the MM position nucleotide, respectively;
(b) a second strand of nucleotides that is complementary to the first strand of nucleotides.

62. The branched oligonucleotide of claim 61, wherein:
the branched oligonucleotide comprises two siRNA molecules covalently bound to one another; or
the siRNA molecules are covalently bound to one another by way of a linker.

63. (canceled)


SEQUENCE LISTING (386 sequences)

SEQ ID NO: 1 - DNA, 41 bp, Homo sapiens
  source 1..41: mol_type = other DNA
gttaagagatggggacagtamttcaacgctagaagaacaca

SEQ ID NO: 2 - DNA, 41 bp, Homo sapiens
  source 1..41: mol_type = other DNA
agccacgagaagctgctgctrcagatcaaccccgagcggga

SEQ ID NO: 3 - DNA, 41 bp, Homo sapiens
  source 1..41: mol_type = other DNA
ccggagcctttggaagtctgygcccttgtgccctgcctcca

SEQ ID NO: 4 - DNA, 41 bp, Homo sapiens
  source 1..41: mol_type = other DNA
cagcccgagctgcctgcagarccggcggcctactggagcaa

SEQ ID NO: 5 - DNA, 41 bp, Homo sapiens
  source 1..41: mol_type = other DNA
cccacgcctgctccctcatcyactgtgtgcacttcatcctg

SEQ ID NO: 6 - DNA, 41 bp, Homo sapiens
  source 1..41: mol_type = other DNA
gggttggagccctgcacggcrtcctctatgtgctggagtgc

SEQ ID NO: 7 - DNA, 41 bp, Homo sapiens
  source 1..41: mol_type = other DNA
ctgctggttgttgccaggttrcagctgctcttgcatctggg

SEQ ID NO: 8 - DNA, 41 bp, Homo sapiens
  source 1..41: mol_type = other DNA
tcctccctcctgcaggctggstgttggcccctstgctgtcc

SEQ ID NO: 9 - DNA, 41 bp, Homo sapiens
  source 1..41: mol_type = other DNA
gatttgggagctctgcttgcygactggctgtgagacgaggc

SEQ ID NO: 10 - DNA, 41 bp, Homo sapiens
  source 1..41: mol_type = other DNA
gaaaagtttggagggtttctycgctcagccttggatgttct

SEQ ID NO: 11 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 12 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 13 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 14 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 15 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 16 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 17 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 18 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 19 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 20 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 21 - RNA, 20 nt, synthetic construct
  misc_feature 1..20: note = Description of Artificial Sequence: Synthetic oligonucleotide
  source 1..20: mol_type = other RNA
tcgcggacttccaaaggctc

SEQ ID NO: 22 - RNA, 20 nt, synthetic construct
  misc_feature 1..20: note = Description of Artificial Sequence: Synthetic oligonucleotide
  source 1..20: mol_type = other RNA
tcgcaggcttccaaaggctc

SEQ ID NO: 23 - RNA, 20 nt, synthetic construct
  misc_feature 1..20: note = Description of Artificial Sequence: Synthetic oligonucleotide
  source 1..20: mol_type = other RNA
tcgcagatttccaaaggctc

SEQ ID NO: 24 - RNA, 15 nt, synthetic construct
  misc_feature 1..15: note = Description of Artificial Sequence: Synthetic oligonucleotide
  source 1..15: mol_type = other RNA
agaagctgctgctaa

SEQ ID NO: 25 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 26 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 27 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 28 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 29 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 30 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 31 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 32 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 33 - RNA, 15 nt, synthetic construct
  misc_feature 1..15: note = Description of Artificial Sequence: Synthetic oligonucleotide
  source 1..15: mol_type = other RNA
tttggaagtccgcga

SEQ ID NO: 34 - RNA, 15 nt, synthetic construct
  misc_feature 1..15: note = Description of Artificial Sequence: Synthetic oligonucleotide
  source 1..15: mol_type = other RNA
tttggaagcctgcga

SEQ ID NO: 35 - RNA, 15 nt, synthetic construct
  misc_feature 1..15: note = Description of Artificial Sequence: Synthetic oligonucleotide
  source 1..15: mol_type = other RNA
tttggaaatctgcga

SEQ ID NO: 36 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 37 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 38 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 39 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 40 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 41 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 42 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 43 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 44 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 45 - RNA, 20 nt, synthetic construct
  misc_feature 1..20: note = Description of Artificial Sequence: Synthetic oligonucleotide
  source 1..20: mol_type = other RNA
tcgcggacttccaaaggctc

SEQ ID NO: 46 - RNA, 20 nt, synthetic construct
  misc_feature 1..20: note = Description of Artificial Sequence: Synthetic oligonucleotide
  source 1..20: mol_type = other RNA
tcgcaggcttccaaaggctc

SEQ ID NO: 47 - RNA, 20 nt, synthetic construct
  misc_feature 1..20: note = Description of Artificial Sequence: Synthetic oligonucleotide
  source 1..20: mol_type = other RNA
tcgcagatttccaaaggctc

SEQ ID NO: 48 - RNA, 15 nt, synthetic construct
  misc_feature 1..15: note = Description of Artificial Sequence: Synthetic oligonucleotide
  source 1..15: mol_type = other RNA
agaagctgctgctaa

SEQ ID NO: 49 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 50 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 51 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 52 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 53 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 54 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 55 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 56 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 57 - RNA, 15 nt, synthetic construct
  misc_feature 1..15: note = Description of Artificial Sequence: Synthetic oligonucleotide
  source 1..15: mol_type = other RNA
tttggaagtccgcga

SEQ ID NO: 58 - RNA, 15 nt, synthetic construct
  misc_feature 1..15: note = Description of Artificial Sequence: Synthetic oligonucleotide
  source 1..15: mol_type = other RNA
tttggaagcctgcga

SEQ ID NO: 59 - RNA, 15 nt, synthetic construct
  misc_feature 1..15: note = Description of Artificial Sequence: Synthetic oligonucleotide
  source 1..15: mol_type = other RNA
tttggaaatctgcga

SEQ ID NO: 60 - RNA, 20 nt, synthetic construct
  misc_feature 1..20: note = Description of Artificial Sequence: Synthetic oligonucleotide
  source 1..20: mol_type = other RNA
tcagcatcagcttctcgtgg

SEQ ID NO: 61 - RNA, 20 nt, synthetic construct
  misc_feature 1..20: note = Description of Artificial Sequence: Synthetic oligonucleotide
  source 1..20: mol_type = other RNA
ttgcagtagcagcttctcgt

SEQ ID NO: 62 - RNA, 20 nt, synthetic construct
  misc_feature 1..20: note = Description of Artificial Sequence: Synthetic oligonucleotide
  source 1..20: mol_type = other RNA
ttgcagctgcagcttctcgt

SEQ ID NO: 63 - RNA, 20 nt, synthetic construct
  misc_feature 1..20: note = Description of Artificial Sequence: Synthetic oligonucleotide
  source 1..20: mol_type = other RNA
ttgcagcagcagctactcgt

SEQ ID NO: 64 - RNA, 20 nt, synthetic construct
  misc_feature 1..20: note = Description of Artificial Sequence: Synthetic oligonucleotide
  source 1..20: mol_type = other RNA
ttctacagcagcagcttctc

SEQ ID NO: 65 - RNA, 20 nt, synthetic construct
  misc_feature 1..20: note = Description of Artificial Sequence: Synthetic oligonucleotide
  source 1..20: mol_type = other RNA
ttctgcatcagcagcttctc

SEQ ID NO: 66 - RNA, 20 nt, synthetic construct
  misc_feature 1..20: note = Description of Artificial Sequence: Synthetic oligonucleotide
  source 1..20: mol_type = other RNA
ttctgcagcatcagcttctc

SEQ ID NO: 67 - RNA, 20 nt, synthetic construct
  misc_feature 1..20: note = Description of Artificial Sequence: Synthetic oligonucleotide
  source 1..20: mol_type = other RNA
ttctgcagcagcatcttctc

SEQ ID NO: 68 - RNA, 20 nt, synthetic construct
  misc_feature 1..20: note = Description of Artificial Sequence: Synthetic oligonucleotide
  source 1..20: mol_type = other RNA
tcacggacttccaaaggctc

SEQ ID NO: 69 - RNA, 20 nt, synthetic construct
  misc_feature 1..20: note = Description of Artificial Sequence: Synthetic oligonucleotide
  source 1..20: mol_type = other RNA
tcacaggcttccaaaggctc

SEQ ID NO: 70 - RNA, 20 nt, synthetic construct
  misc_feature 1..20: note = Description of Artificial Sequence: Synthetic oligonucleotide
  source 1..20: mol_type = other RNA
tcacagatttccaaaggctc

SEQ ID NO: 71 - RNA, 15 nt, synthetic construct
  misc_feature 1..15: note = Description of Artificial Sequence: Synthetic oligonucleotide
  source 1..15: mol_type = other RNA
agaagctgctgctga

SEQ ID NO: 72 - RNA, 15 nt, synthetic construct
  misc_feature 1..15: note = Description of Artificial Sequence: Synthetic oligonucleotide
  source 1..15: mol_type = other RNA
aagctgctgctgcaa

SEQ ID NO: 73 - RNA, 15 nt, synthetic construct
  misc_feature 1..15: note = Description of Artificial Sequence: Synthetic oligonucleotide
  source 1..15: mol_type = other RNA
aagctgctgctgcaa

SEQ ID NO: 74 - RNA, 15 nt, synthetic construct
  misc_feature 1..15: note = Description of Artificial Sequence: Synthetic oligonucleotide
  source 1..15: mol_type = other RNA
aagctgctgctgcaa

SEQ ID NO: 75 - RNA, 15 nt, synthetic construct
  misc_feature 1..15: note = Description of Artificial Sequence: Synthetic oligonucleotide
  source 1..15: mol_type = other RNA
gctgctgctgcagaa

SEQ ID NO: 76 - RNA, 15 nt, synthetic construct
  misc_feature 1..15: note = Description of Artificial Sequence: Synthetic oligonucleotide
  source 1..15: mol_type = other RNA
gctgctgctgcagaa

SEQ ID NO: 77 - RNA, 15 nt, synthetic construct
  misc_feature 1..15: note = Description of Artificial Sequence: Synthetic oligonucleotide
  source 1..15: mol_type = other RNA
gctgctgctgcagaa

SEQ ID NO: 78 - RNA, 15 nt, synthetic construct
  misc_feature 1..15: note = Description of Artificial Sequence: Synthetic oligonucleotide
  source 1..15: mol_type = other RNA
gctgctgctgcagaa

SEQ ID NO: 79 - RNA, 15 nt, synthetic construct
  misc_feature 1..15: note = Description of Artificial Sequence: Synthetic oligonucleotide
  source 1..15: mol_type = other RNA
tttggaagtccgtga

SEQ ID NO: 80 - RNA, 15 nt, synthetic construct
  misc_feature 1..15: note = Description of Artificial Sequence: Synthetic oligonucleotide
  source 1..15: mol_type = other RNA
tttggaagcctgtga

SEQ ID NO: 81 - RNA, 15 nt, synthetic construct
  misc_feature 1..15: note = Description of Artificial Sequence: Synthetic oligonucleotide
  source 1..15: mol_type = other RNA
tttggaaatctgtga

SEQ ID NO: 82 - DNA, 45 bp, synthetic construct
  misc_feature 1..45: note = Description of Artificial Sequence: Synthetic oligonucleotide
  source 1..45: mol_type = other DNA
tcgaagccacgagaagctgctgctacagatcaaccccgagcggga

SEQ ID NO: 83 - DNA, 45 bp, synthetic construct
  misc_feature 1..45: note = Description of Artificial Sequence: Synthetic oligonucleotide
  source 1..45: mol_type = other DNA
ggcctcccgctcggggttgatctgtagcagcagcttctcgtggct

SEQ ID NO: 84 - DNA, 45 bp, synthetic construct
  misc_feature 1..45: note = Description of Artificial Sequence: Synthetic oligonucleotide
  source 1..45: mol_type = other DNA
tcgaagccacgagaagctgctgctgcagatcaaccccgagcggga

SEQ ID NO: 85 - DNA, 45 bp, synthetic construct
  misc_feature 1..45: note = Description of Artificial Sequence: Synthetic oligonucleotide
  source 1..45: mol_type = other DNA
ggcctcccgctcggggttgatctgcagcagcagcttctcgtggct

SEQ ID NO: 86 - RNA, 20 nt, synthetic construct
  misc_feature 1..20: note = Description of Artificial Sequence: Synthetic oligonucleotide
  source 1..20: mol_type = other RNA
ttggcagcagcttctcgtgg

SEQ ID NO: 87 - RNA, 20 nt, synthetic construct
  misc_feature 1..20: note = Description of Artificial Sequence: Synthetic oligonucleotide
  source 1..20: mol_type = other RNA
tttgcagcagcttctcgtgg

SEQ ID NO: 88 - RNA, 20 nt, synthetic construct
  misc_feature 1..20: note = Description of Artificial Sequence: Synthetic oligonucleotide
  source 1..20: mol_type = other RNA
ttcgcagcagcttctcgtgg

SEQ ID NO: 89 - RNA, 20 nt, synthetic construct
  misc_feature 1..20: note = Description of Artificial Sequence: Synthetic oligonucleotide
  source 1..20: mol_type = other RNA
ttatcagcagcttctcgtgg

SEQ ID NO: 90 - RNA, 20 nt, synthetic construct
  misc_feature 1..20: note = Description of Artificial Sequence: Synthetic oligonucleotide
  source 1..20: mol_type = other RNA
ttagtagcagcttctcgtgg

SEQ ID NO: 91 - RNA, 20 nt, synthetic construct
  misc_feature 1..20: note = Description of Artificial Sequence: Synthetic oligonucleotide
  source 1..20: mol_type = other RNA
ttagctgcagcttctcgtgg

SEQ ID NO: 92 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 93 - RNA, 20 nt, synthetic construct
  misc_feature 1..20: note = Description of Artificial Sequence: Synthetic oligonucleotide
  source 1..20: mol_type = other RNA
ttagcagtagcttctcgtgg

SEQ ID NO: 94 - RNA, 20 nt, synthetic construct
  misc_feature 1..20: note = Description of Artificial Sequence: Synthetic oligonucleotide
  source 1..20: mol_type = other RNA
ttagcagctgcttctcgtgg

SEQ ID NO: 95 - RNA, 20 nt, synthetic construct
  misc_feature 1..20: note = Description of Artificial Sequence: Synthetic oligonucleotide
  source 1..20: mol_type = other RNA
ttagcagcatcttctcgtgg

SEQ ID NO: 96 - RNA, 20 nt, synthetic construct
  misc_feature 1..20: note = Description of Artificial Sequence: Synthetic oligonucleotide
  source 1..20: mol_type = other RNA
ttagcagcagtttctcgtgg

SEQ ID NO: 97 - RNA, 20 nt, synthetic construct
  misc_feature 1..20: note = Description of Artificial Sequence: Synthetic oligonucleotide
  source 1..20: mol_type = other RNA
ttagcagcagcatctcgtgg

SEQ ID NO: 98 - RNA, 20 nt, synthetic construct
  misc_feature 1..20: note = Description of Artificial Sequence: Synthetic oligonucleotide
  source 1..20: mol_type = other RNA
ttagcagcagctactcgtgg

SEQ ID NO: 99 - RNA, 20 nt, synthetic construct
  misc_feature 1..20: note = Description of Artificial Sequence: Synthetic oligonucleotide
  source 1..20: mol_type = other RNA
ttagcagcagcttttcgtgg

SEQ ID NO: 100 - RNA, 20 nt, synthetic construct
  misc_feature 1..20: note = Description of Artificial Sequence: Synthetic oligonucleotide
  source 1..20: mol_type = other RNA
ttagcagcagcttcacgtgg

SEQ ID NO: 101 - RNA, 20 nt, synthetic construct
  misc_feature 1..20: note = Description of Artificial Sequence: Synthetic oligonucleotide
  source 1..20: mol_type = other RNA
ttagcagcagcttcttgtgg

SEQ ID NO: 102 - RNA, 20 nt, synthetic construct
  misc_feature 1..20: note = Description of Artificial Sequence: Synthetic oligonucleotide
  source 1..20: mol_type = other RNA
tagtagcagcagcttctcgt

SEQ ID NO: 103 - RNA, 20 nt, synthetic construct
  misc_feature 1..20: note = Description of Artificial Sequence: Synthetic oligonucleotide
  source 1..20: mol_type = other RNA
ttttagcagcagcttctcgt

SEQ ID NO: 104 - RNA, 20 nt, synthetic construct
  misc_feature 1..20: note = Description of Artificial Sequence: Synthetic oligonucleotide
  source 1..20: mol_type = other RNA
ttatagcagcagcttctcgt

SEQ ID NO: 105 - RNA, 20 nt, synthetic construct
  misc_feature 1..20: note = Description of Artificial Sequence: Synthetic oligonucleotide
  source 1..20: mol_type = other RNA
ttgtcgcagcagcttctcgt

SEQ ID NO: 106 - RNA, 20 nt, synthetic construct
  misc_feature 1..20: note = Description of Artificial Sequence: Synthetic oligonucleotide
  source 1..20: mol_type = other RNA
ttgtggcagcagcttctcgt

SEQ ID NO: 107 - RNA, 20 nt, synthetic construct
  misc_feature 1..20: note = Description of Artificial Sequence: Synthetic oligonucleotide
  source 1..20: mol_type = other RNA
ttgtatcagcagcttctcgt

SEQ ID NO: 108 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 109 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 110 - RNA, 20 nt, synthetic construct
  misc_feature 1..20: note = Description of Artificial Sequence: Synthetic oligonucleotide
  source 1..20: mol_type = other RNA
ttgtagcatcagcttctcgt

SEQ ID NO: 111 - RNA, 20 nt, synthetic construct
  misc_feature 1..20: note = Description of Artificial Sequence: Synthetic oligonucleotide
  source 1..20: mol_type = other RNA
ttgtagcagtagcttctcgt

SEQ ID NO: 112 - RNA, 20 nt, synthetic construct
  misc_feature 1..20: note = Description of Artificial Sequence: Synthetic oligonucleotide
  source 1..20: mol_type = other RNA
ttgtagcagctgcttctcgt

SEQ ID NO: 113 - RNA, 20 nt, synthetic construct
  misc_feature 1..20: note = Description of Artificial Sequence: Synthetic oligonucleotide
  source 1..20: mol_type = other RNA
ttgtagcagcatcttctcgt

SEQ ID NO: 114 - RNA, 20 nt, synthetic construct
  misc_feature 1..20: note = Description of Artificial Sequence: Synthetic oligonucleotide
  source 1..20: mol_type = other RNA
ttgtagcagcagtttctcgt

SEQ ID NO: 115 - RNA, 20 nt, synthetic construct
  misc_feature 1..20: note = Description of Artificial Sequence: Synthetic oligonucleotide
  source 1..20: mol_type = other RNA
ttgtagcagcagcatctcgt

SEQ ID NO: 116 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 117 - RNA, 20 nt, synthetic construct
  misc_feature 1..20: note = Description of Artificial Sequence: Synthetic oligonucleotide
  source 1..20: mol_type = other RNA
ttgtagcagcagcttttcgt

SEQ ID NO: 118 - RNA, 20 nt, synthetic construct
  misc_feature 1..20: note = Description of Artificial Sequence: Synthetic oligonucleotide
  source 1..20: mol_type = other RNA
tactgtagcagcagcttctc

SEQ ID NO: 119 - RNA, 20 nt, synthetic construct
  misc_feature 1..20: note = Description of Artificial Sequence: Synthetic oligonucleotide
  source 1..20: mol_type = other RNA
ttttgtagcagcagcttctc

SEQ ID NO: 120 - RNA, 20 nt, synthetic construct
  misc_feature 1..20: note = Description of Artificial Sequence: Synthetic oligonucleotide
  source 1..20: mol_type = other RNA
ttcagtagcagcagcttctc

SEQ ID NO: 121 - RNA, 20 nt, synthetic construct
  misc_feature 1..20: note = Description of Artificial Sequence: Synthetic oligonucleotide
  source 1..20: mol_type = other RNA
ttctttagcagcagcttctc

SEQ ID NO: 122 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 123 - RNA, 20 nt, synthetic construct
  misc_feature 1..20: note = Description of Artificial Sequence: Synthetic oligonucleotide
  source 1..20: mol_type = other RNA
ttctgtcgcagcagcttctc

SEQ ID NO: 124 - RNA, 20 nt, synthetic construct
  misc_feature 1..20: note = Description of Artificial Sequence: Synthetic oligonucleotide
  source 1..20: mol_type = other RNA
ttctgtggcagcagcttctc

SEQ ID NO: 125 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 126 - RNA, 20 nt, synthetic construct
  misc_feature 1..20: note = Description of Artificial Sequence: Synthetic oligonucleotide
  source 1..20: mol_type = other RNA
ttctgtagtagcagcttctc

SEQ ID NO: 127 - RNA, 20 nt, synthetic construct
  misc_feature 1..20: note = Description of Artificial Sequence: Synthetic oligonucleotide
  source 1..20: mol_type = other RNA
ttctgtagctgcagcttctc

SEQ ID NO: 128 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 129 - RNA, 20 nt, synthetic construct
  misc_feature 1..20: note = Description of Artificial Sequence: Synthetic oligonucleotide
  source 1..20: mol_type = other RNA
ttctgtagcagtagcttctc

SEQ ID NO: 130 - RNA, 20 nt, synthetic construct
  misc_feature 1..20: note = Description of Artificial Sequence: Synthetic oligonucleotide
  source 1..20: mol_type = other RNA
ttctgtagcagctgcttctc

SEQ ID NO: 131 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 132 - RNA, 20 nt, synthetic construct
  misc_feature 1..20: note = Description of Artificial Sequence: Synthetic oligonucleotide
  source 1..20: mol_type = other RNA
ttctgtagcagcagtttctc

SEQ ID NO: 133 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 134 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 135 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 136 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 137 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 138 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 139 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 140 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 141 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 142 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 143 - RNA, 15 nt, synthetic construct
  misc_feature 1..15: note = Description of Artificial Sequence: Synthetic oligonucleotide
  source 1..15: mol_type = other RNA
agaagctgctgctaa

SEQ ID NO: 144 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 145 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 146 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 147 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 148 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 149 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 150 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 151 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 152 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 153 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 154 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 155 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 156 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 157 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 158 - RNA, 20 nt, synthetic construct
  misc_feature 1..20: note = Description of Artificial Sequence: Synthetic oligonucleotide
  source 1..20: mol_type = other RNA
tctctttactgatataatta

SEQ ID NO: 159 - RNA, 20 nt, synthetic construct
  misc_feature 1..20: note = Description of Artificial Sequence: Synthetic oligonucleotide
  source 1..20: mol_type = other RNA
tatgttttcacatattgtca

SEQ ID NO: 160 - RNA, 20 nt, synthetic construct
  misc_feature 1..20: note = Description of Artificial Sequence: Synthetic oligonucleotide
  source 1..20: mol_type = other RNA
tgaatgttcacgcagtgggc

SEQ ID NO: 161 - RNA, 20 nt, synthetic construct
  misc_feature 1..20: note = Description of Artificial Sequence: Synthetic oligonucleotide
  source 1..20: mol_type = other RNA
tatcagcttttccagggtcg

SEQ ID NO: 162 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 163 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 164 - RNA, 20 nt, synthetic construct
  misc_feature 1..20: note = Description of Artificial Sequence: Synthetic oligonucleotide
  source 1..20: mol_type = other RNA
ttaacgtcagttcataaacc

SEQ ID NO: 165 - RNA, 20 nt, synthetic construct
  misc_feature 1..20: note = Description of Artificial Sequence: Synthetic oligonucleotide
  source 1..20: mol_type = other RNA
tccactatgttttcacatat

SEQ ID NO: 166 - RNA, 20 nt, synthetic construct
  misc_feature 1..20: note = Description of Artificial Sequence: Synthetic oligonucleotide
  source 1..20: mol_type = other RNA
tccaaatactggttgtcggt

SEQ ID NO: 167 - RNA, 20 nt, synthetic construct
  misc_feature 1..20: note = Description of Artificial Sequence: Synthetic oligonucleotide
  source 1..20: mol_type = other RNA
tccggtcacaacattgtggt

SEQ ID NO: 168 - RNA, 20 nt, synthetic construct
  misc_feature 1..20: note = Description of Artificial Sequence: Synthetic oligonucleotide
  source 1..20: mol_type = other RNA
tatgttttcacatattgtca

SEQ ID NO: 169 - RNA, 20 nt, synthetic construct
  misc_feature 1..20: note = Description of Artificial Sequence: Synthetic oligonucleotide
  source 1..20: mol_type = other RNA
tttggtagctgaaagttctt

SEQ ID NO: 170 - RNA, 20 nt, synthetic construct
  misc_feature 1..20: note = Description of Artificial Sequence: Synthetic oligonucleotide
  source 1..20: mol_type = other RNA
ttaatctctttactgattta

SEQ ID NO: 171 - RNA, 20 nt, synthetic construct
  misc_feature 1..20: note = Description of Artificial Sequence: Synthetic oligonucleotide
  source 1..20: mol_type = other RNA
tctctttactgatataatta

SEQ ID NO: 172 - RNA, 20 nt, synthetic construct
  misc_feature 1..20: note = Description of Artificial Sequence: Synthetic oligonucleotide
  source 1..20: mol_type = other RNA
ttaatctctttactgatatt

SEQ ID NO: 173 - RNA, 20 nt, synthetic construct
  misc_feature 1..20: note = Description of Artificial Sequence: Synthetic oligonucleotide
  source 1..20: mol_type = other RNA
ttaatctctttactgatttt

SEQ ID NO: 174 - RNA, 20 nt, synthetic construct
  misc_feature 1..20: note = Description of Artificial Sequence: Synthetic oligonucleotide
  source 1..20: mol_type = other RNA
tactgtagcagcagcttctc

SEQ ID NO: 175 - RNA, 20 nt, synthetic construct
  misc_feature 1..20: note = Description of Artificial Sequence: Synthetic oligonucleotide
  source 1..20: mol_type = other RNA
ttttgtagcagcagcttctc

SEQ ID NO: 176 - RNA, 20 nt, synthetic construct
  misc_feature 1..20: note = Description of Artificial Sequence: Synthetic oligonucleotide
  source 1..20: mol_type = other RNA
ttcagtagcagcagcttctc

SEQ ID NO: 177 - RNA, 20 nt, synthetic construct
  misc_feature 1..20: note = Description of Artificial Sequence: Synthetic oligonucleotide
  source 1..20: mol_type = other RNA
ttctttagcagcagcttctc

SEQ ID NO: 178 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 179 - RNA, 20 nt, synthetic construct
  misc_feature 1..20: note = Description of Artificial Sequence: Synthetic oligonucleotide
  source 1..20: mol_type = other RNA
ttctgtcgcagcagcttctc

SEQ ID NO: 180 - RNA, 20 nt, synthetic construct
  misc_feature 1..20: note = Description of Artificial Sequence: Synthetic oligonucleotide
  source 1..20: mol_type = other RNA
ttctgtggcagcagcttctc

SEQ ID NO: 181 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 182 - RNA, 20 nt, synthetic construct
  misc_feature 1..20: note = Description of Artificial Sequence: Synthetic oligonucleotide
  source 1..20: mol_type = other RNA
ttctgtagtagcagcttctc

SEQ ID NO: 183 - RNA, 20 nt, synthetic construct
  misc_feature 1..20: note = Description of Artificial Sequence: Synthetic oligonucleotide
  source 1..20: mol_type = other RNA
ttctgtagctgcagcttctc

SEQ ID NO: 184 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 185 - RNA, 20 nt, synthetic construct
  misc_feature 1..20: note = Description of Artificial Sequence: Synthetic oligonucleotide
  source 1..20: mol_type = other RNA
ttctgtagcagtagcttctc

SEQ ID NO: 186 - RNA, 20 nt, synthetic construct
  misc_feature 1..20: note = Description of Artificial Sequence: Synthetic oligonucleotide
  source 1..20: mol_type = other RNA
ttctgtagcagctgcttctc

SEQ ID NO: 187 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 188 - RNA, 20 nt, synthetic construct
  misc_feature 1..20: note = Description of Artificial Sequence: Synthetic oligonucleotide
  source 1..20: mol_type = other RNA
ttctgtagcagcagtttctc

SEQ ID NO: 189 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 190 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 191 - RNA, 23 nt, synthetic construct
  misc_feature 1..23: note = Description of Artificial Sequence: Synthetic oligonucleotide
  source 1..23: mol_type = other RNA
gagaagctgctgctgctacagaa

SEQ ID NO: 192 - RNA, 23 nt, synthetic construct
  misc_feature 1..23: note = Description of Artificial Sequence: Synthetic oligonucleotide
  source 1..23: mol_type = other RNA
gagaagctgctgctgctgcagaa

SEQ ID NO: 193 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 194 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 195 - RNA, 20 nt, synthetic construct
  misc_feature 1..20: note = Description of Artificial Sequence: Synthetic oligonucleotide
  source 1..20: mol_type = other RNA
ttctgtagcaacagcttctc

SEQ ID NO: 196 - RNA, 20 nt, synthetic construct
  misc_feature 1..20: note = Description of Artificial Sequence: Synthetic oligonucleotide
  source 1..20: mol_type = other RNA
ttctgtagcaccagcttctc

SEQ ID NO: 197 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 198 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 199 - RNA, 20 nt, synthetic construct
  misc_feature 1..20: note = Description of Artificial Sequence: Synthetic oligonucleotide
  source 1..20: mol_type = other RNA
ttctgtagcaacagcttctc

SEQ ID NO: 200 - RNA, 20 nt, synthetic construct
  misc_feature 1..20: note = Description of Artificial Sequence: Synthetic oligonucleotide
  source 1..20: mol_type = other RNA
ttctgtagcaccagcttctc

SEQ ID NO: 201 - RNA, 23 nt, synthetic construct
  misc_feature 1..23: note = Description of Artificial Sequence: Synthetic oligonucleotide
  source 1..23: mol_type = other RNA
gagaagctgctgctgctacagaa

SEQ ID NO: 202 - RNA, 23 nt, synthetic construct
  misc_feature 1..23: note = Description of Artificial Sequence: Synthetic oligonucleotide
  source 1..23: mol_type = other RNA
gagaagctgctgctgctgcagaa

SEQ ID NO: 203 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 204 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 205 - RNA, 20 nt, synthetic construct
  misc_feature 1..20: note = Description of Artificial Sequence: Synthetic oligonucleotide
  source 1..20: mol_type = other RNA
ttctgtagcaacagcttctc

SEQ ID NO: 206 - RNA, 20 nt, synthetic construct
  misc_feature 1..20: note = Description of Artificial Sequence: Synthetic oligonucleotide
  source 1..20: mol_type = other RNA
ttctgtagcaccagcttctc

SEQ ID NO: 207 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 208 - RNA, 15 nt, synthetic construct
  misc_feature 1..15: note = Description of Artificial Sequence: Synthetic oligonucleotide
  source 1..15: mol_type = other RNA
aattagagaaatgac

SEQ ID NO: 209 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 210 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 211 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 212 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 213 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 214 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 215 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 216 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 217 - RNA, 20 nt, synthetic construct
  misc_feature 1..20: note = Description of Artificial Sequence: Synthetic oligonucleotide
  source 1..20: mol_type = other RNA
ttagcagcagcttctcgtgg

SEQ ID NO: 218 - RNA, 20 nt, synthetic construct
  misc_feature 1..20: note = Description of Artificial Sequence: Synthetic oligonucleotide
  source 1..20: mol_type = other RNA
tgtagcagcagcttctcgtg

SEQ ID NO: 219 - RNA, 20 nt, synthetic construct
  misc_feature 1..20: note = Description of Artificial Sequence: Synthetic oligonucleotide
  source 1..20: mol_type = other RNA
ttgtagcagcagcttctcgt

SEQ ID NO: 220 - RNA, 20 nt, synthetic construct
  misc_feature 1..20: note = Description of Artificial Sequence: Synthetic oligonucleotide
  source 1..20: mol_type = other RNA
tctgtagcagcagcttctcg

SEQ ID NO: 221 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 222 - RNA, 20 nt, synthetic construct
  misc_feature 1..20: note = Description of Artificial Sequence: Synthetic oligonucleotide
  source 1..20: mol_type = other RNA
tatctgtagcagcagcttct

SEQ ID NO: 223 - RNA, 20 nt, synthetic construct
  misc_feature 1..20: note = Description of Artificial Sequence: Synthetic oligonucleotide
  source 1..20: mol_type = other RNA
tgatctgtagcagcagcttc

SEQ ID NO: 224 - RNA, 20 nt, synthetic construct
  misc_feature 1..20: note = Description of Artificial Sequence: Synthetic oligonucleotide
  source 1..20: mol_type = other RNA
ttgatctgtagcagcagctt

SEQ ID NO: 225 - RNA, 20 nt, synthetic construct
  misc_feature 1..20: note = Description of Artificial Sequence: Synthetic oligonucleotide
  source 1..20: mol_type = other RNA
tttgatctgtagcagcagct

SEQ ID NO: 226 - RNA, 20 nt, synthetic construct
  misc_feature 1..20: note = Description of Artificial Sequence: Synthetic oligonucleotide
  source 1..20: mol_type = other RNA
tgttgatctgtagcagcagc

SEQ ID NO: 227 - RNA, 20 nt, synthetic construct
  misc_feature 1..20: note = Description of Artificial Sequence: Synthetic oligonucleotide
  source 1..20: mol_type = other RNA
tggttgatctgtagcagcag

SEQ ID NO: 228 - RNA, 20 nt, synthetic construct
  misc_feature 1..20: note = Description of Artificial Sequence: Synthetic oligonucleotide
  source 1..20: mol_type = other RNA
tgggttgatctgtagcagca

SEQ ID NO: 229 - DNA, 105 bp, Homo sapiens
  misc_feature 1..105: note = This sequence may encompass 8-35 'cag' repeating units
  source 1..105: mol_type = other DNA
cagcagcagcagcagcagcagcagcagcagcagcagcagcagcagcagcagcagcagcagcagcagcagcagcagcagcagcagcagcagcagcagcagcagcag

SEQ ID NO: 230 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 231 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 232 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 233 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 234 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 235 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 236 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 237 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 238 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 239 - RNA, 20 nt, synthetic construct
  misc_feature 1..20: note = Description of Artificial Sequence: Synthetic oligonucleotide
  source 1..20: mol_type = other RNA
tcgcggacttccaaaggctc

SEQ ID NO: 240 - RNA, 20 nt, synthetic construct
  misc_feature 1..20: note = Description of Artificial Sequence: Synthetic oligonucleotide
  source 1..20: mol_type = other RNA
tcgcaggcttccaaaggctc

SEQ ID NO: 241 - RNA, 20 nt, synthetic construct
  misc_feature 1..20: note = Description of Artificial Sequence: Synthetic oligonucleotide
  source 1..20: mol_type = other RNA
tcgcagatttccaaaggctc

SEQ ID NO: 242 - RNA, 15 nt, synthetic construct
  misc_feature 1..15: note = Description of Artificial Sequence: Synthetic oligonucleotide
  source 1..15: mol_type = other RNA
agaagctgctgctaa

SEQ ID NO: 243 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 244 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 245 - RNA, 15 nt, synthetic construct
  misc_feature 1..15: note = Description of Artificial Sequence: Synthetic oligonucleotide
  source 1..15: mol_type = other RNA
tttggaagtccgcga

SEQ ID NO: 246 - RNA, 15 nt, synthetic construct
  misc_feature 1..15: note = Description of Artificial Sequence: Synthetic oligonucleotide
  source 1..15: mol_type = other RNA
tttggaagcctgcga

SEQ ID NO: 247 - RNA, 15 nt, synthetic construct
  misc_feature 1..15: note = Description of Artificial Sequence: Synthetic oligonucleotide
  source 1..15: mol_type = other RNA
tttggaaatctgcga

SEQ ID NO: 248 - RNA, 20 nt, synthetic construct
  misc_feature 1..20: note = Description of Artificial Sequence: Synthetic oligonucleotide
  source 1..20: mol_type = other RNA
tcagcatcagcttctcgtgg

SEQ ID NO: 249 - RNA, 20 nt, synthetic construct
  misc_feature 1..20: note = Description of Artificial Sequence: Synthetic oligonucleotide
  source 1..20: mol_type = other RNA
ttgcagtagcagcttctcgt

SEQ ID NO: 250 - RNA, 20 nt, synthetic construct
  misc_feature 1..20: note = Description of Artificial Sequence: Synthetic oligonucleotide
  source 1..20: mol_type = other RNA
ttgcagctgcagcttctcgt

SEQ ID NO: 251 - RNA, 20 nt, synthetic construct
  misc_feature 1..20: note = Description of Artificial Sequence: Synthetic oligonucleotide
  source 1..20: mol_type = other RNA
ttgcagcagcagctactcgt

SEQ ID NO: 252 - RNA, 20 nt, synthetic construct
  misc_feature 1..20: note = Description of Artificial Sequence: Synthetic oligonucleotide
  source 1..20: mol_type = other RNA
ttctacagcagcagcttctc

SEQ ID NO: 253 - RNA, 20 nt, synthetic construct
  misc_feature 1..20: note = Description of Artificial Sequence: Synthetic oligonucleotide
  source 1..20: mol_type = other RNA
ttctgcatcagcagcttctc

SEQ ID NO: 254 - RNA, 20 nt, synthetic construct
  misc_feature 1..20: note = Description of Artificial Sequence: Synthetic oligonucleotide
  source 1..20: mol_type = other RNA
ttctgcagcatcagcttctc

SEQ ID NO: 255 - RNA, 20 nt, synthetic construct
  misc_feature 1..20: note = Description of Artificial Sequence: Synthetic oligonucleotide
  source 1..20: mol_type = other RNA
ttctgcagcagcatcttctc

SEQ ID NO: 256 - RNA, 20 nt, synthetic construct
  misc_feature 1..20: note = Description of Artificial Sequence: Synthetic oligonucleotide
  source 1..20: mol_type = other RNA
tcacggacttccaaaggctc

SEQ ID NO: 257 - RNA, 20 nt, synthetic construct
  misc_feature 1..20: note = Description of Artificial Sequence: Synthetic oligonucleotide
  source 1..20: mol_type = other RNA
tcacaggcttccaaaggctc

SEQ ID NO: 258 - RNA, 20 nt, synthetic construct
  misc_feature 1..20: note = Description of Artificial Sequence: Synthetic oligonucleotide
  source 1..20: mol_type = other RNA
tcacagatttccaaaggctc

SEQ ID NO: 259 - RNA, 15 nt, synthetic construct
  misc_feature 1..15: note = Description of Artificial Sequence: Synthetic oligonucleotide
  source 1..15: mol_type = other RNA
agaagctgctgctga

SEQ ID NO: 260 - RNA, 15 nt, synthetic construct
  misc_feature 1..15: note = Description of Artificial Sequence: Synthetic oligonucleotide
  source 1..15: mol_type = other RNA
aagctgctgctgcaa

SEQ ID NO: 261 - RNA, 15 nt, synthetic construct
  misc_feature 1..15: note = Description of Artificial Sequence: Synthetic oligonucleotide
  source 1..15: mol_type = other RNA
gctgctgctgcagaa

SEQ ID NO: 262 - RNA, 15 nt, synthetic construct
  misc_feature 1..15: note = Description of Artificial Sequence: Synthetic oligonucleotide
  source 1..15: mol_type = other RNA
tttggaagtccgtga

SEQ ID NO: 263 - RNA, 15 nt, synthetic construct
  misc_feature 1..15: note = Description of Artificial Sequence: Synthetic oligonucleotide
  source 1..15: mol_type = other RNA
tttggaagcctgtga

SEQ ID NO: 264 - RNA, 15 nt, synthetic construct
  misc_feature 1..15: note = Description of Artificial Sequence: Synthetic oligonucleotide
  source 1..15: mol_type = other RNA
tttggaaatctgtga

SEQ ID NO: 265 - DNA, 45 bp, synthetic construct; identical to SEQ ID NO: 82
  misc_feature 1..45: note = Description of Artificial Sequence: Synthetic oligonucleotide
  source 1..45: mol_type = other DNA

SEQ ID NO: 266 - DNA, 45 bp, synthetic construct; identical to SEQ ID NO: 83
  misc_feature 1..45: note = Description of Artificial Sequence: Synthetic oligonucleotide
  source 1..45: mol_type = other DNA

SEQ ID NO: 267 - DNA, 45 bp, synthetic construct; identical to SEQ ID NO: 84
  misc_feature 1..45: note = Description of Artificial Sequence: Synthetic oligonucleotide
  source 1..45: mol_type = other DNA

SEQ ID NO: 268 - DNA, 45 bp, synthetic construct; identical to SEQ ID NO: 85
  misc_feature 1..45: note = Description of Artificial Sequence: Synthetic oligonucleotide
  source 1..45: mol_type = other DNA

SEQ ID NO: 269 - RNA, 20 nt, synthetic construct
  misc_feature 1..20: note = Description of Artificial Sequence: Synthetic oligonucleotide
  source 1..20: mol_type = other RNA
ttggcagcagcttctcgtgg

SEQ ID NO: 270 - RNA, 20 nt, synthetic construct
  misc_feature 1..20: note = Description of Artificial Sequence: Synthetic oligonucleotide
  source 1..20: mol_type = other RNA
tttgcagcagcttctcgtgg

SEQ ID NO: 271 - RNA, 20 nt, synthetic construct
  misc_feature 1..20: note = Description of Artificial Sequence: Synthetic oligonucleotide
  source 1..20: mol_type = other RNA
ttcgcagcagcttctcgtgg

SEQ ID NO: 272 - RNA, 20 nt, synthetic construct
  misc_feature 1..20: note = Description of Artificial Sequence: Synthetic oligonucleotide
  source 1..20: mol_type = other RNA
ttgtcagcagcttctcgtgg

SEQ ID NO: 273 - RNA, 20 nt, synthetic construct
  misc_feature 1..20: note = Description of Artificial Sequence: Synthetic oligonucleotide
  source 1..20: mol_type = other RNA
ttggtagcagcttctcgtgg

SEQ ID NO: 274 - RNA, 20 nt, synthetic construct
  misc_feature 1..20: note = Description of Artificial Sequence: Synthetic oligonucleotide
  source 1..20: mol_type = other RNA
ttggctgcagcttctcgtgg

SEQ ID NO: 275 - RNA, 20 nt, synthetic construct
  misc_feature 1..20: note = Description of Artificial Sequence: Synthetic oligonucleotide
  source 1..20: mol_type = other RNA
ttggtatcagcttctcgtgg

SEQ ID NO: 276 - RNA, 20 nt, synthetic construct
  misc_feature 1..20: note = Description of Artificial Sequence: Synthetic oligonucleotide
  source 1..20: mol_type = other RNA
ttggcagtagcttctcgtgg

SEQ ID NO: 277 - RNA, 20 nt, synthetic construct
  misc_feature 1..20: note = Description of Artificial Sequence: Synthetic oligonucleotide
  source 1..20: mol_type = other RNA
ttggccgctgcttctcgtgg

SEQ ID NO: 278 - RNA, 20 nt, synthetic construct
  misc_feature 1..20: note = Description of Artificial Sequence: Synthetic oligonucleotide
  source 1..20: mol_type = other RNA
ttggccgcatcttctcgtgg

SEQ ID NO: 279 - RNA, 20 nt, synthetic construct
  misc_feature 1..20: note = Description of Artificial Sequence: Synthetic oligonucleotide
  source 1..20: mol_type = other RNA
ttggccgcagtttctcgtgg

SEQ ID NO: 280 - RNA, 20 nt, synthetic construct
  misc_feature 1..20: note = Description of Artificial Sequence: Synthetic oligonucleotide
  source 1..20: mol_type = other RNA
ttggccgcagcatctcgtgg

SEQ ID NO: 281 - RNA, 20 nt, synthetic construct
  misc_feature 1..20: note = Description of Artificial Sequence: Synthetic oligonucleotide
  source 1..20: mol_type = other RNA
ttggccgcagctactcgtgg

SEQ ID NO: 282 - RNA, 20 nt, synthetic construct
  misc_feature 1..20: note = Description of Artificial Sequence: Synthetic oligonucleotide
  source 1..20: mol_type = other RNA
ttggccgcagcttttcgtgg

SEQ ID NO: 283 - RNA, 20 nt, synthetic construct
  misc_feature 1..20: note = Description of Artificial Sequence: Synthetic oligonucleotide
  source 1..20: mol_type = other RNA
ttggccgcagcttcacgtgg

SEQ ID NO: 284 - RNA, 20 nt, synthetic construct
  misc_feature 1..20: note = Description of Artificial Sequence: Synthetic oligonucleotide
  source 1..20: mol_type = other RNA
ttggccgcagcttcttgtgg

SEQ ID NO: 285 - RNA, 20 nt, synthetic construct
  misc_feature 1..20: note = Description of Artificial Sequence: Synthetic oligonucleotide
  source 1..20: mol_type = other RNA
tagtagcagcagcttctcgt

SEQ ID NO: 286 - RNA, 20 nt, synthetic construct
  misc_feature 1..20: note = Description of Artificial Sequence: Synthetic oligonucleotide
  source 1..20: mol_type = other RNA
ttttagcagcagcttctcgt

SEQ ID NO: 287 - RNA, 20 nt, synthetic construct
  misc_feature 1..20: note = Description of Artificial Sequence: Synthetic oligonucleotide
  source 1..20: mol_type = other RNA
ttatagcagcagcttctcgt

SEQ ID NO: 288 - RNA, 20 nt, synthetic construct
  misc_feature 1..20: note = Description of Artificial Sequence: Synthetic oligonucleotide
  source 1..20: mol_type = other RNA
ttgtcgcagcagcttctcgt

SEQ ID NO: 289 - RNA, 20 nt, synthetic construct
  misc_feature 1..20: note = Description of Artificial Sequence: Synthetic oligonucleotide
  source 1..20: mol_type = other RNA
ttgtggcagcagcttctcgt

SEQ ID NO: 290 - RNA, 20 nt, synthetic construct
  misc_feature 1..20: note = Description of Artificial Sequence: Synthetic oligonucleotide
  source 1..20: mol_type = other RNA
ttgtatcagcagcttctcgt

SEQ ID NO: 291 - RNA, 20 nt, synthetic construct
  misc_feature 1..20: note = Description of Artificial Sequence: Synthetic oligonucleotide
  source 1..20: mol_type = other RNA
ttgtagtagcagcttctcgt

SEQ ID NO: 292 - RNA, 20 nt, synthetic construct
  misc_feature 1..20: note = Description of Artificial Sequence: Synthetic oligonucleotide
  source 1..20: mol_type = other RNA
ttgtagctgcagcttctcgt

SEQ ID NO: 293 - RNA, 20 nt, synthetic construct
  misc_feature 1..20: note = Description of Artificial Sequence: Synthetic oligonucleotide
  source 1..20: mol_type = other RNA
ttgtagcatcagcttctcgt

SEQ ID NO: 294 - RNA, 20 nt, synthetic construct
  misc_feature 1..20: note = Description of Artificial Sequence: Synthetic oligonucleotide
  source 1..20: mol_type = other RNA
ttgtagcagtagcttctcgt

SEQ ID NO: 295 - RNA, 20 nt, synthetic construct
  misc_feature 1..20: note = Description of Artificial Sequence: Synthetic oligonucleotide
  source 1..20: mol_type = other RNA
ttgtagcagctgcttctcgt

SEQ ID NO: 296 - RNA, 20 nt, synthetic construct
  misc_feature 1..20: note = Description of Artificial Sequence: Synthetic oligonucleotide
  source 1..20: mol_type = other RNA
ttgtagcagcatcttctcgt

SEQ ID NO: 297 - RNA, 20 nt, synthetic construct
  misc_feature 1..20: note = Description of Artificial Sequence: Synthetic oligonucleotide
  source 1..20: mol_type = other RNA
ttgtagcagcagtttctcgt

SEQ ID NO: 298 - RNA, 20 nt, synthetic construct
  misc_feature 1..20: note = Description of Artificial Sequence: Synthetic oligonucleotide
  source 1..20: mol_type = other RNA
ttgtagcagcagcatctcgt

SEQ ID NO: 299 - RNA, 20 nt, synthetic construct
  misc_feature 1..20: note = Description of Artificial Sequence: Synthetic oligonucleotide
  source 1..20: mol_type = other RNA
ttgtagcagcagctactcgt

SEQ ID NO: 300 - RNA, 20 nt, synthetic construct
  misc_feature 1..20: note = Description of Artificial Sequence: Synthetic oligonucleotide
  source 1..20: mol_type = other RNA
ttgtagcagcagcttttcgt

SEQ ID NO: 301 - RNA, 20 nt, synthetic construct
  misc_feature 1..20: note = Description of Artificial Sequence: Synthetic oligonucleotide
  source 1..20: mol_type = other RNA
tactgtagcagcagcttctc

SEQ ID NO: 302 - RNA, 20 nt, synthetic construct
  misc_feature 1..20: note = Description of Artificial Sequence: Synthetic oligonucleotide
  source 1..20: mol_type = other RNA
ttttgtagcagcagcttctc

SEQ ID NO: 303 - RNA, 20 nt, synthetic construct
  misc_feature 1..20: note = Description of Artificial Sequence: Synthetic oligonucleotide
  source 1..20: mol_type = other RNA
ttcagtagcagcagcttctc

SEQ ID NO: 304 - RNA, 20 nt, synthetic construct
  misc_feature 1..20: note = Description of Artificial Sequence: Synthetic oligonucleotide
  source 1..20: mol_type = other RNA
ttctttagcagcagcttctc

SEQ ID NO: 305 - RNA, 20 nt, synthetic construct
  misc_feature 1..20: note = Description of Artificial Sequence: Synthetic oligonucleotide
  source 1..20: mol_type = other RNA
ttctatagcagcagcttctc

SEQ ID NO: 306 - RNA, 20 nt, synthetic construct
  misc_feature 1..20: note = Description of Artificial Sequence: Synthetic oligonucleotide
  source 1..20: mol_type = other RNA
ttctgtcgcagcagcttctc

SEQ ID NO: 307 - RNA, 20 nt, synthetic construct
  misc_feature 1..20: note = Description of Artificial Sequence: Synthetic oligonucleotide
  source 1..20: mol_type = other RNA
ttctgtggcagcagcttctc

SEQ ID NO: 308 - RNA, 20 nt, synthetic construct
  misc_feature 1..20: note = Description of Artificial Sequence: Synthetic oligonucleotide
  source 1..20: mol_type = other RNA
ttctgtatcagcagcttctc

SEQ ID NO: 309 - RNA, 20 nt, synthetic construct
  misc_feature 1..20: note = Description of Artificial Sequence: Synthetic oligonucleotide
  source 1..20: mol_type = other RNA
ttctgtagtagcagcttctc

SEQ ID NO: 310 - RNA, 20 nt, synthetic construct
  misc_feature 1..20: note = Description of Artificial Sequence: Synthetic oligonucleotide
  source 1..20: mol_type = other RNA
ttctgtagctgcagcttctc

SEQ ID NO: 311 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 312 - RNA, 20 nt, synthetic construct
  misc_feature 1..20: note = Description of Artificial Sequence: Synthetic oligonucleotide
  source 1..20: mol_type = other RNA
ttctgtagcagtagcttctc

SEQ ID NO: 313 - RNA, 20 nt, synthetic construct
  misc_feature 1..20: note = Description of Artificial Sequence: Synthetic oligonucleotide
  source 1..20: mol_type = other RNA
ttctgtagcagctgcttctc

SEQ ID NO: 314 - RNA, 20 nt, synthetic construct
  misc_feature 1..20: note = Description of Artificial Sequence: Synthetic oligonucleotide
  source 1..20: mol_type = other RNA
ttctgtagcagcatcttctc

SEQ ID NO: 315 - RNA, 20 nt, synthetic construct
  misc_feature 1..20: note = Description of Artificial Sequence: Synthetic oligonucleotide
  source 1..20: mol_type = other RNA
ttctgtagcagcagtttctc

SEQ ID NO: 316 - RNA, 20 nt, synthetic construct
  misc_feature 1..20: note = Description of Artificial Sequence: Synthetic oligonucleotide
  source 1..20: mol_type = other RNA
ttctgtagcagcagcatctc

SEQ ID NO: 317 - RNA, 20 nt, synthetic construct
  misc_feature 1..20: note = Description of Artificial Sequence: Synthetic oligonucleotide
  source 1..20: mol_type = other RNA
ttagcatcagcttctcgtgg

SEQ ID NO: 318 - RNA, 15 nt, synthetic construct
  misc_feature 1..15: note = Description of Artificial Sequence: Synthetic oligonucleotide
  source 1..15: mol_type = other RNA
agaagctgctgctaa

SEQ ID NO: 319 - RNA, 15 nt, synthetic construct
  misc_feature 1..15: note = Description of Artificial Sequence: Synthetic oligonucleotide
  source 1..15: mol_type = other RNA
aagctgctgctacaa

SEQ ID NO: 320 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 321 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 322 - RNA, 15 nt, synthetic construct
  misc_feature 1..15: note = Description of Artificial Sequence: Synthetic oligonucleotide
  source 1..15: mol_type = other RNA
gctgctgctacagaa

SEQ ID NO: 323 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 324 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 325 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 326 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 327 - RNA, 20 nt, synthetic construct
  misc_feature 1..20: note = Description of Artificial Sequence: Synthetic oligonucleotide
  source 1..20: mol_type = other RNA
tctctttactgatataatta

SEQ ID NO: 328 - RNA, 20 nt, synthetic construct
  misc_feature 1..20: note = Description of Artificial Sequence: Synthetic oligonucleotide
  source 1..20: mol_type = other RNA
tatcttttcacatattgtca

SEQ ID NO: 329 - RNA, 20 nt, synthetic construct
  misc_feature 1..20: note = Description of Artificial Sequence: Synthetic oligonucleotide
  source 1..20: mol_type = other RNA
tgaatgttcacgcagtgggc

SEQ ID NO: 330 - RNA, 20 nt, synthetic construct
  misc_feature 1..20: note = Description of Artificial Sequence: Synthetic oligonucleotide
  source 1..20: mol_type = other RNA
tatcagcttttccagggtcg

SEQ ID NO: 331 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 332 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 333 - RNA, 20 nt, synthetic construct
  misc_feature 1..20: note = Description of Artificial Sequence: Synthetic oligonucleotide
  source 1..20: mol_type = other RNA
ttaacgtcagttcataaacc

SEQ ID NO: 334 - RNA, 20 nt, synthetic construct
  misc_feature 1..20: note = Description of Artificial Sequence: Synthetic oligonucleotide
  source 1..20: mol_type = other RNA
tccactatgttttcacatat

SEQ ID NO: 335 - RNA, 20 nt, synthetic construct
  misc_feature 1..20: note = Description of Artificial Sequence: Synthetic oligonucleotide
  source 1..20: mol_type = other RNA
tccaaatactggttgtcggt

SEQ ID NO: 336 - RNA, 20 nt, synthetic construct
  misc_feature 1..20: note = Description of Artificial Sequence: Synthetic oligonucleotide
  source 1..20: mol_type = other RNA
tccggtcacaacattgtggt

SEQ ID NO: 337 - RNA, 20 nt, synthetic construct
  misc_feature 1..20: note = Description of Artificial Sequence: Synthetic oligonucleotide
  source 1..20: mol_type = other RNA
tatgttttcacatattgtca

SEQ ID NO: 338 - RNA, 20 nt, synthetic construct
  misc_feature 1..20: note = Description of Artificial Sequence: Synthetic oligonucleotide
  source 1..20: mol_type = other RNA
tttggtagctgaaagttctt

SEQ ID NO: 339 - RNA, 20 nt, synthetic construct
  misc_feature 1..20: note = Description of Artificial Sequence: Synthetic oligonucleotide
  source 1..20: mol_type = other RNA
ttaatctctttactgattta

SEQ ID NO: 340 - RNA, 20 nt, synthetic construct
  misc_feature 1..20: note = Description of Artificial Sequence: Synthetic oligonucleotide
  source 1..20: mol_type = other RNA
tctctttactgatataatta

SEQ ID NO: 341 - RNA, 20 nt, synthetic construct
  misc_feature 1..20: note = Description of Artificial Sequence: Synthetic oligonucleotide
  source 1..20: mol_type = other RNA
ttaatctctttactgatatt

SEQ ID NO: 342 - RNA, 20 nt, synthetic construct
  misc_feature 1..20: note = Description of Artificial Sequence: Synthetic oligonucleotide
  source 1..20: mol_type = other RNA
ttaatctctttactgatttt

SEQ ID NO: 343 - RNA, 20 nt, synthetic construct
  misc_feature 1..20: note = Description of Artificial Sequence: Synthetic oligonucleotide
  source 1..20: mol_type = other RNA
tactgtagcagcagcttctc

SEQ ID NO: 344 - RNA, 20 nt, synthetic construct
  misc_feature 1..20: note = Description of Artificial Sequence: Synthetic oligonucleotide
  source 1..20: mol_type = other RNA
ttttgtagcagcagcttctc

SEQ ID NO: 345 - RNA, 20 nt, synthetic construct
  misc_feature 1..20: note = Description of Artificial Sequence: Synthetic oligonucleotide
  source 1..20: mol_type = other RNA
ttcagtagcagcagcttctc

SEQ ID NO: 346 - RNA, 20 nt, synthetic construct
  misc_feature 1..20: note = Description of Artificial Sequence: Synthetic oligonucleotide
  source 1..20: mol_type = other RNA
ttctttagcagcagcttctc

SEQ ID NO: 347 - RNA, 20 nt, synthetic construct
  misc_feature 1..20: note = Description of Artificial Sequence: Synthetic oligonucleotide
  source 1..20: mol_type = other RNA
ttctgtcgcagcagcttctc

SEQ ID NO: 348 - RNA, 20 nt, synthetic construct
  misc_feature 1..20: note = Description of Artificial Sequence: Synthetic oligonucleotide
  source 1..20: mol_type = other RNA
ttctgtggcagcagcttctc

SEQ ID NO: 349 - RNA, 20 nt, synthetic construct
  misc_feature 1..20: note = Description of Artificial Sequence: Synthetic oligonucleotide
  source 1..20: mol_type = other RNA
ttctgtagtagcagcttctc

SEQ ID NO: 350 - RNA, 20 nt, synthetic construct
  misc_feature 1..20: note = Description of Artificial Sequence: Synthetic oligonucleotide
  source 1..20: mol_type = other RNA
ttctgtagctgcagcttctc

SEQ ID NO: 351 - RNA, 20 nt, synthetic construct
  misc_feature 1..20: note = Description of Artificial Sequence: Synthetic oligonucleotide
  source 1..20: mol_type = other RNA
ttctgtcgcagtagcttctc

SEQ ID NO: 352 - RNA, 20 nt, synthetic construct
  misc_feature 1..20: note = Description of Artificial Sequence: Synthetic oligonucleotide
  source 1..20: mol_type = other RNA
ttctgtagcagctgcttctc

SEQ ID NO: 353 - RNA, 20 nt, synthetic construct
  misc_feature 1..20: note = Description of Artificial Sequence: Synthetic oligonucleotide
  source 1..20: mol_type = other RNA
ttctgtagcagcagtttctc

SEQ ID NO: 354 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 355 - RNA, 23 nt, synthetic construct
  misc_feature 1..23: note = Description of Artificial Sequence: Synthetic oligonucleotide
  source 1..23: mol_type = other RNA
gagaagctgctgctgctacagaa

SEQ ID NO: 356 - RNA, 23 nt, synthetic construct
  misc_feature 1..23: note = Description of Artificial Sequence: Synthetic oligonucleotide
  source 1..23: mol_type = other RNA
gagaagctgctgctgctgcagaa

SEQ ID NO: 357 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 358 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 359 - RNA, 20 nt, synthetic construct
  misc_feature 1..20: note = Description of Artificial Sequence: Synthetic oligonucleotide
  source 1..20: mol_type = other RNA
ttctgtagcaacagcttctc

SEQ ID NO: 360 - RNA, 20 nt, synthetic construct
  misc_feature 1..20: note = Description of Artificial Sequence: Synthetic oligonucleotide
  source 1..20: mol_type = other RNA
ttctgtagcaccagcttctc

SEQ ID NO: 361 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 362 - RNA, 20 nt, synthetic construct
  misc_feature 1..20: note = Description of Artificial Sequence: Synthetic oligonucleotide
  source 1..20: mol_type = other RNA
ttctgtagcatcagcttctc

SEQ ID NO: 363 - RNA, 20 nt, synthetic construct
  misc_feature 1..20: note = Description of Artificial Sequence: Synthetic oligonucleotide
  source 1..20: mol_type = other RNA
ttctgtagcaacagcttctc

SEQ ID NO: 364 - RNA, 20 nt, synthetic construct
  misc_feature 1..20: note = Description of Artificial Sequence: Synthetic oligonucleotide
  source 1..20: mol_type = other RNA
ttctgtagcaccagcttctc

SEQ ID NO: 365 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 366 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 367 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 368 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 369 - RNA, 20 nt, synthetic construct
  misc_feature 1..20: note = Description of Artificial Sequence: Synthetic oligonucleotide
  source 1..20: mol_type = other RNA
ttttggataaactggtagcc

SEQ ID NO: 370 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 371 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 372 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 373 - RNA, 15 nt, synthetic construct
  misc_feature 1..15: note = Description of Artificial Sequence: Synthetic oligonucleotide
  source 1..15: mol_type = other RNA
ccagtttatccaaaa

SEQ ID NO: 374 - RNA, 20 nt, synthetic construct
  misc_feature 1..20: note = Description of Artificial Sequence: Synthetic oligonucleotide
  source 1..20: mol_type = other RNA
ttagcagcagcttctcgtgg

SEQ ID NO: 375 - RNA, 20 nt, synthetic construct
  misc_feature 1..20: note = Description of Artificial Sequence: Synthetic oligonucleotide
  source 1..20: mol_type = other RNA
tgtagcagcagcttctcgtg

SEQ ID NO: 376 - RNA, 20 nt, synthetic construct
  misc_feature 1..20: note = Description of Artificial Sequence: Synthetic oligonucleotide
  source 1..20: mol_type = other RNA
ttgtagcagcagcttctcgt

SEQ ID NO: 377 - RNA, 20 nt, synthetic construct
  misc_feature 1..20: note = Description of Artificial Sequence: Synthetic oligonucleotide
  source 1..20: mol_type = other RNA
tctgtagcagcagcttctcg

SEQ ID NO: 378 - RNA, 20 nt, synthetic construct
  misc_feature 1..20: note = Description of Artificial Sequence: Synthetic oligonucleotide
  source 1..20: mol_type = other RNA
ttctgtagcagcagcttctc

SEQ ID NO: 379 - RNA, 20 nt, synthetic construct
  misc_feature 1..20: note = Description of Artificial Sequence: Synthetic oligonucleotide
  source 1..20: mol_type = other RNA
tatctgtagcagcagcttct

SEQ ID NO: 380 - RNA, 20 nt, synthetic construct
  misc_feature 1..20: note = Description of Artificial Sequence: Synthetic oligonucleotide
  source 1..20: mol_type = other RNA
tgatctgtagcagcagcttc

SEQ ID NO: 381 - RNA, 20 nt, synthetic construct
  misc_feature 1..20: note = Description of Artificial Sequence: Synthetic oligonucleotide
  source 1..20: mol_type = other RNA
ttgatctgtagcagcagctt

SEQ ID NO: 382 - RNA, 20 nt, synthetic construct
  misc_feature 1..20: note = Description of Artificial Sequence: Synthetic oligonucleotide
  source 1..20: mol_type = other RNA
tttgatctgtagcagcagct

SEQ ID NO: 383 - RNA, 20 nt, synthetic construct
  misc_feature 1..20: note = Description of Artificial Sequence: Synthetic oligonucleotide
  source 1..20: mol_type = other RNA
tgttgatctgtagcagcagc

SEQ ID NO: 384 - RNA, 20 nt, synthetic construct
  misc_feature 1..20: note = Description of Artificial Sequence: Synthetic oligonucleotide
  source 1..20: mol_type = other RNA
tggttgatctgtagcagcag

SEQ ID NO: 385 - RNA, 20 nt, synthetic construct
  misc_feature 1..20: note = Description of Artificial Sequence: Synthetic oligonucleotide
  source 1..20: mol_type = other RNA
tgggttgatctgtagcagca

SEQ ID NO: 386 - RNA, 15 nt, synthetic construct
  misc_feature 1..15: note = Description of Artificial Sequence: Synthetic oligonucleotide
  source 1..15: mol_type = other RNA
cagtaaagagattaa